(19) United States
(12) Patent Application Publication
(10) Pub. No.: US 2026/0217743 A1
(43) Pub. Date: Jul. 30, 2026

(54) ETHER OF PHOSPHATIDYLCHOLINE AND PHOSPHATIDYLETHANOLAMINE, THEIR LYSO-FORMS AND MIXTURE THEREOF FOR USE IN THE TREATMENT OF A DISEASE OR DISORDER CAUSED BY A DEFICIENCY IN PLASMALOGENS

(71) Applicants: Baker Heart and Diabetes Institute, Melbourne (AU); Pelagic AI Limited, Dublin 2 (IE)

(72) Inventors: Peter J. Meikle, Melbourne (AU); Paul Sudip, Melbourne (AU); Amy Boileau, Libertyville, IL (US); Martin Ducker, Oxford (GB); Kevin Huynh, Melbourne (AU)

(21) Appl. No.: 19/145,456

(22) PCT Filed: Jan. 3, 2024
(86) PCT No.: PCT/US2024/010129

§ 371 Date: Jul. 2, 2025

Related U.S. Application Data
(60) Provisional application No. 63/478,289, filed on Jan. 3, 2023.
(60) Provisional application No. 63/589,539, filed on Oct. 11, 2023.

(51) Int. Cl. C07F 9/10; A23L 33/00; A61K 8/55; A61K 31/661; A61Q 11/00

(52) U.S. Cl. CPC C07F 9/10; A23L 33/40; A61K 8/553; A61K 31/661; A61Q 11/00

(57) ABSTRACT

The disclosure relates to certain compounds that can be consumed by a human subject to modulate the levels of plasmalogens in the subject to provide improved health outcomes. The disclosure further relates to the formulation of said compounds, compositions comprising at least one compound, and uses thereof.

### CROSS-REFERENCE TO RELATED APPLICATIONS

This application claims priority to U.S. Provisional Patent Application No. 63/478,289, filed on Jan. 3, 2023, and to U.S. Provisional Patent Application No. 63/589,539, filed on Oct. 11, 2023, the entire contents of each of which are hereby incorporated by reference in their entirety.

### TECHNICAL FIELD

This disclosure relates to certain precursor compounds such as lysoalkylphosphatidylcholines, (LPC(O)s) that can be consumed by a human subject to increase the levels of plasmalogens in the subject to provide improved health outcomes. More specifically, this disclosure relates to the formulation of the precursor compounds to provide optimal elevation of circulating and tissue plasmalogens while maintaining an optimal composition of plasmalogen species.

### BACKGROUND

Plasmalogens, as defined herein, comprise a group of plasmenyl-phospholipids which are a major cell membrane component. As part of the broader class of phospholipids, plasmanyl- and/or plasmenyl-phospholipids are a unique class of ether phospholipids that are major components of cell membranes. Their biophysical role in cell membranes has been studied while knowledge concerning their biological roles is an important area of new research. Plasmalogens are primarily present as alkenylphosphatidylcholine (PC) and alkenylphosphatidylethanolamine (PE) species, a.k.a. PC(P) and PE(P), respectively. They are characterized by a cis-vinyl ether bond linking an alkyl chain to the sn-1 carbon position of the glycerol backbone. They also optionally have an acyl linked fatty acid ester bond in the sn-2 carbon position. Plasmalogens are often esterified with polyunsaturated fatty acids such as arachidonic acid (20:4) and the omega-3 fatty acid docosahexaenoic acid (22:6, a major constituent of fish oil), whereas the vinyl ether linked residue is usually otherwise saturated (i.e., no double bonds are present in the chain other than the vinyl ether group) or otherwise monounsaturated (i.e., one double bond is present in the chain in addition to the vinyl ether group).

Plasmalogen biosynthesis is a complex process involving multiple enzymes within the peroxisome and endoplasmic reticulum. The rate-limiting step in this pathway is the formation of the long chain fatty alcohol by fatty acyl-CoA reductase 1 and 2 (Far-1/2). It is possible to bypass the rate-limiting step in plasmalogen synthesis through the oral administration of naturally occurring alkylglycerols (1-O-alkylglycerol or 1-O-alkyl-2,3-diacylglycerol). These can be incorporated directly into the phospholipid pathway, and so bypass the peroxisome. This leads to an increase in circulating and tissue plasmalogens. Although alkylglycerols are present in our diet, the levels in typical diets are insufficient to significantly boost human plasmalogen levels. Shark liver oil is rich in alkylglycerols and is currently used as a dietary supplement to reduce inflammation and improve immune function. Alkylglycerols can also be synthesized, providing a future avenue for an environmentally sustainable source of these compounds (Magnusson, C. D., et al., Tetrahedron (2011) 67:1821-36; Shi, Y., et al., Green Chemistry (2010) 12(12)).

WO 2021/007623 A1 to Baker Heart and Diabetes Institute, which is incorporated by reference herein in its entirety, relates generally to various compositions and methods for maintaining or modulating mixtures of ether lipid molecules in human tissues, used for non-disease conditions and for treating certain disease conditions including obesity, diabetes, fatty liver disease, cardiovascular disease and Alzheimer's disease.

There exists an unmet need for an improved mixture or composition comprising precursor compounds for plasmalogens, which would serve as an important contribution to one or more fields including, but not limited to, the medicinal, pharmaceutical, dietary supplement, and nutritional arts.

### SUMMARY

In one embodiment, a formulation, which may take the form of one or more of a medicament, a pharmaceutical, a dietary supplement, and a food product or other nutritional composition, is described, said formulation comprising, consisting of, or consisting essentially of one or more ether lipid compounds. It has been found that the ingestion of lysoalkylphosphatidylcholine, a.k.a. LPC(O), and alkylphosphatidylcholine, a.k.a. PC(O), in krill oil led to the elevation of PE(P) and PC(P) species. This is not obvious as it is generally considered that LPC(O) and PC(O) are a dead end on the plasmalogen biosynthetic pathway and that there is no active pathway for the conversion of LPC(O) and PC(O) into PE(P) and PC(P). Thus, it appears that the LPC(O)/PC(O) are able to be shunted into the plasmalogen biosynthetic pathway and/or they are able to directly stimulate the natural biosynthetic pathway to increase plasmalogen synthesis.

Comparison of the increase in the level of plasmalogen in circulation (plasma) after a given treatment of LPC(O) and PC(O) (krill oil) with the elevation following a similar treatment of AKDAG (shark liver oil) identified that the LPC(O) and PC(O) were more bioavailable. That is, it was surprisingly discovered that a smaller dose of LPC(O) and PC(O) could lead to the same increase in circulating levels of plasmalogens as would be obtained by administering a comparatively larger dose of AKDAG (shark liver oil).

It is notable that phospholipids found in krill oil differ substantially from that in humans; however, there is an expectation that LPC(O) levels are connected to plasmalogens, or at the least, are involved with the modulation of plasmalogens in humans.

Additionally, it was recognized that the composition of the alkyl chains in LPC(O) and PC(O) in krill oil was substantially different from the composition of alkenyl chains in plasmalogens in humans and so could potentially influence the composition of the resulting plasmalogens as had been previously reported for alkylglycerol (AKG) and alkyldiacylglycerol (AKDAG) (WO 2021/007623). This led to the present development and disclosure of a formulation of LPC(O), and a prophetic formulation of lysoalkylphosphatidylethanolamine, a.k.a. LPE(O), or lysoalkylphosphatidic acid, a.k.a. LPA(O), that would match the known plasmalogen composition in human plasma, as described herein.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 describes changes in plasma ether lipid classes (% change) from baseline/pre-treatment following krill oil (KO), fish oil (FO) or shark liver oil (SLO) supplementation. Changes in plasma ether lipid classes were grouped by supplement. Subjects showed significant percent changes in ether lipid classes after KO (measured at 15 and 30 days) and SLO treatments (measured at 21 days). FO supplementation did not have a significant impact on plasma ether lipid classes. The nominal significance of the treatment effect was determined using paired t-tests (Krill oil baseline vs Krill oil 15 days, Krill oil baseline vs Krill oil 30 days, Fish oil baseline vs Fish oil 15 days, Fish oil baseline vs Fish oil 30 days; SLO pre-treatment vs SLO 21 days); * indicates P<0.05, ** indicates P<0.01 and *** indicates P<0.001.

FIG. 2 describes changes in plasma ether lipid classes (% change) from baseline/pre-treatment following KO, FO or SLO supplementation (grouped by lipid class). The effects of different supplements were grouped by plasma ether lipid classes. Subjects showed significant percent changes in ether lipid classes after KO (measured at 15 and 30 days) and SLO treatments (measured at 21 days). FO supplementation did not have a significant impact on plasma ether lipid classes. The nominal significance of the treatment effect was determined using paired t-tests (Krill oil baseline vs Krill oil 15 days, Krill oil baseline vs Krill oil 30 days, Fish oil baseline vs Fish oil 15 days, Fish oil baseline vs Fish oil 30 days; SLO pre-treatment vs SLO 21 days); * indicates P<0.05, ** indicates P<0.01 and *** indicates P<0.001.

FIG. 3 describes the effect of KO supplementation on alkenyl chain composition of PE plasmalogens. Data are presented as Mean±SD. KO supplementation did not have a significant impact on alkenyl chain composition of PE plasmalogen. The nominal significance of the treatment effect was determined using Repeated Measures ANOVA followed by Tukey's multiple comparison tests; * indicated P<0.05, ** indicates P<0.01 and *** indicates P<0.001.

FIG. 4 describes the effect of FO supplementation on alkenyl chain composition of PE plasmalogens. Data are presented as Mean±SD. FO supplementation did not have a significant impact on alkenyl chain composition of PE plasmalogen. The nominal significance of the treatment effect was determined using Repeated Measures ANOVA followed by Tukey's multiple comparison tests; * indicated P<0.05.

FIG. 5 describes the effect of SLO supplementation on alkenyl chain composition of PE plasmalogens. Data are presented as Mean±SD. SLO supplementation significantly altered the alkenyl chain composition of PE plasmalogen. There was an increase in the proportion of 18:1 alkenyl chain containing PE plasmalogens and a decrease in the proportions of 16:0 and 18:0 alkenyl chain containing PE plasmalogens. The nominal significance of the treatment effect was determined using paired t-tests; ** indicates P<0.01 and *** indicates P<0.001.

FIG. 6 describes the effect of KO supplementation on acyl chain composition of PE plasmalogens. Data are presented as Mean±SD. KO supplementation decreased the proportion of 18:1, 18:2 and 20:4 containing PE plasmalogens and increased the proportions of 20:5 and 22:6 containing PE plasmalogens. The nominal significance of the treatment effect was determined using Repeated Measures ANOVA followed by Tukey's multiple comparison tests; ** indicates P<0.01 and *** indicates P<0.001.

FIG. 7 describes the effect of FO supplementation on acyl chain composition of PE plasmalogens. Data are presented as Mean±SD. FO supplementation decreased the proportion of 18:1, 18:2 and 20:4 containing PE plasmalogens and increased the proportions of 20:5 and 22:6 containing PE plasmalogens. The nominal significance of the treatment effect was determined using Repeated Measures ANOVA followed by Tukey's multiple comparison tests; * indicated P<0.05, ** indicates P<0.01 and *** indicates P<0.001.

FIG. 8 describes the effect of SLO supplementation on acyl chain composition of PE plasmalogens. Data are presented as Mean±SD. SLO supplementation decreased 20:4 containing PE plasmalogens and increased the proportions of 18:1 and 22:6 containing PE plasmalogens. The nominal significance of the treatment effect was determined using paired t-tests; ** indicates P<0.01 and *** indicates P<0.001.

FIG. 9 shows HepG2 cells before and after plasmalogen precursor treatment. Cells were treated with 0.05% ethanol (vehicle control) or labelled plasmalogen precursors at 20 μM for 24 hours. Labelled precursors included alkylglycerol (O-16:0)d2 (AKG), LPC(O-16:0)d4 (LPC(O)), LPE(O-16:0)d5 (LPE(O)). Images were taken with an Olympus CKX41 Inverted Microscope at 4× magnification and analysed using ImageJ. Scale bar for all images is 400 μm.

FIG. 10 shows labelled phosphatidylethanolamine plasmalogen (PE(P)) concentration after plasmalogen precursor treatment in HepG2 cells. FIG. 10A shows a bar chart displays of labelled PE(P) concentration of the 16:0, 18:0 and 18:1 species. FIG. 10B shows labelled PE(P)/total label (%). Total label refers to the sum of labelled AKG, alkyl-acylglycerol (DG(O)), lysoalkylphosphatidylcholine (LPC(O)), lysophosphatidylcholine plasmalogen (LPC(P)), lysoalkylphosphatidylethanolamine (LPE(O)), lysophosphatidylethanolamine plasmalogen (LPE(P)), alkylphosphatidylcholine (PC(O)), phosphatidylcholine plasmalogen (PC(P)), alkylphosphatidylethanolamine (PE(O)), PE(P), monoalkyl-diacylglycerol (TG(O)). FIG. 10 shows mean±standard deviation (n=3 per group) with each symbol representing an individual sample. FIG. 10B excluded control as it divides a background value by a background value, which could be misleading. One-way ANOVA with Tukey's post-hoc test was used. ns indicates p>0.05, ** p≤0.01, *** p≤0.001, **** p≤0.0001.

FIG. 11 shows label incorporation into lipid classes of the plasmalogen biosynthesis pathway after plasmalogen precursor treatment in HepG2 cells. Cells were treated with 0.05% ethanol (vehicle control) or labelled plasmalogen precursors at 20 μM for 24 hours. Labelled precursors included alkylglycerol (O-16:0)d2 (AKG), LPC(O-16:0)d4 (LPC(O)), LPE(O-16:0)d5 (LPE(O)). Concentrations were normalised to phosphatidylcholine (PC) 34:1, one of the most abundant endogenous PC. FIG. 11A shows a bar chart displays label concentration for AKG treatment. FIG. 11B shows a bar chart displays label concentration for LPC(O) treatment. FIG. 11C shows a bar chart displays label concentration for LPE(O) treatment. FIG. 11 shows mean±standard deviation (n=3 per group) with each symbol representing an individual sample.

FIG. 12 shows the distribution of label incorporation in the plasmalogen biosynthesis pathway after plasmalogen precursor treatment in HepG2 cells. Cells were treated with 0.05% ethanol (vehicle control) or labelled plasmalogen precursors at 20 μM for 24 hours. Labelled precursors included alkylglycerol (O-16:0)d2 (AKG), LPC(O-16:0)d4 (LPC(O)), LPE(O-16:0)d5 (LPE(O)). FIG. 12A shows label concentration of each lipid class divided by total label as a percentage for AKG treatment. FIG. 12B shows label concentration of each lipid class divided by total label as a percentage for LPC(O) treatment. FIG. 12C shows label concentration of each lipid class divided by total label as a percentage for LPE(O) treatment. Total label refers to the sum of labelled AKG, alkyl-acylglycerol (DG(O)), lysoalkylphosphatidylcholine (LPC(O)), lysophosphatidylcholine plasmalogen (LPC(P)), lysoalkylphosphatidylethanolamine (LPE(O)), lysophosphatidylethanolamine plasmalogen (LPE(P)), alkylphosphatidylcholine (PC(O)), phosphatidylcholine plasmalogen (PC(P)), alkylphosphatidylethanolamine (PE(O)), phosphatidylethanolamine plasmalogen (PE(P)), monoalkyl-diacylglycerol (TG(O)).

FIG. 13 shows 3T3 cells before and after plasmalogen precursor treatment. Cells were treated with 0.05% ethanol (vehicle control) or labelled plasmalogen precursors at 20 μM for 24 hours. Labelled precursors included alkylglycerol (O-16:0)d2 (AKG), LPC(O-16:0)d4 (LPC(O)), LPE(O-16:0)d5 (LPE(O)). Images were taken with an Olympus CKX41 Inverted Microscope at 4× magnification and analysed using ImageJ. Scale bar for all images is 400 m.

FIG. 14 shows labelled phosphatidylethanolamine plasmalogen (PE(P)) concentration after plasmalogen precursor treatment in 3T3 cells. Concentrations were normalised to phosphatidylcholine (PC) 34:1, one of the most abundant endogenous PC. FIG. 14A shows a bar chart displays of labelled PE(P) concentration of the 16:0, 18:0 and 18:1 species. FIG. 14B shows a bar chart displaying log(2) of labelled PE(P) concentration of the 16:0, 18:0 and 18:1 species. FIG. 14C shows labelled PE(P)/total label (%). Total label refers to the sum of labelled AKG, alkyl-acylglycerol (DG(O)), lysoalkylphosphatidylcholine (LPC(O)), lysophosphatidylcholine plasmalogen (LPC(P)), lysoalkylphosphatidylethanolamine (LPE(O)), lysophosphatidylethanolamine plasmalogen (LPE(P)), alkylphosphatidylcholine (PC(O)), phosphatidylcholine plasmalogen (PC(P)), alkylphosphatidylethanolamine (PE(O)), PE(P), monoalkyl-diacylglycerol (TG(O)). FIG. 14 shows mean±standard deviation (n=3 per group) with each symbol representing an individual sample. FIG. 14C excluded control as it divides a background value by a background value, which could be misleading. One-way ANOVA with Tukey's post-hoc test was used. ns indicates p>0.05, ** p≤0.01, *** p≤0.001, **** p≤0.0001.

FIG. 15 shows label incorporation into lipid classes of the plasmalogen biosynthesis pathway after plasmalogen precursor treatment in 3T3 cells. Cells were treated with 0.05% ethanol (vehicle control) or labelled plasmalogen precursors at 20 μM for 24 hours. Labelled precursors included alkylglycerol (O-16:0)d2 (AKG), LPC(O-16:0)d4 (LPC(O)), LPE(O-16:0)d5 (LPE(O)). Concentrations were normalized to phosphatidylcholine (PC) 34:1, one of the most abundant endogenous PC. FIG. 15A shows a bar chart displays label concentration for AKG treatment. FIG. 15B shows a bar chart displays label concentration for LPC(O) treatment. FIG. 15C shows a bar chart displays label concentration for LPE(O) treatment. FIG. 15 shows mean standard deviation (n=3 per group) with each symbol representing an individual sample.

FIG. 16 shows the distribution of label incorporation in the plasmalogen biosynthesis pathway after plasmalogen precursor treatment in 3T3 cells. Cells were treated with 0.05% ethanol (vehicle control) or labelled plasmalogen precursors at 20 μM for 24 hours. Labelled precursors included alkylglycerol (O-16:0)d2 (AKG), LPC(O-16:0)d4 (LPC(O)), LPE(O-16:0)d5 (LPE(O)). FIG. 16A shows label concentration of each lipid class divided by total label as a percentage for AKG treatment. FIG. 16B shows label concentration of each lipid class divided by total label as a percentage for LPC(O) treatment. FIG. 16C shows label concentration of each lipid class divided by total label as a percentage for LPE(O) treatment.

FIG. 17 describes the procedure for dosing mixtures of deuterated precursor molecules on 64 eight-week-old C57BL/6 mice.

FIG. 18 shows labelled alkylglycerol (AKG) concentration after dose mix A of plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 18A shows a chart displaying labelled AKG O-16:0 concentration at 0, 1, 4, 24 and 48 timepoints for dose mix A of treatment in females. FIG. 18B shows a bar chart displaying labelled AKG O-16:0 concentration at 0, 1, 4, 24 and 48 timepoints for dose mix A of treatment in males. FIG. 18 shows mean±standard deviation (n=4-5 per group) with each symbol representing an individual sample.

FIG. 19 shows labelled alkylphosphatidylcholine (PC(O)) concentration after dose mix A of plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 19A shows a chart displaying labelled PC(O) 16:0 species concentration at 0, 1, 4, 24 and 48 timepoints for dose mix A of treatment in females. FIG. 19B shows a chart displaying labelled PC(O) 16:0 species concentration at 0, 1, 4, 24 and 48 timepoints for dose mix A of treatment in males. FIG. 19 shows mean±standard deviation (n=4-5 per group) with each symbol representing an individual sample.

FIG. 20 shows labelled alkylphosphatidylethanolamine (PE(O)) concentration after dose mix A of plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 20A shows a chart displaying labelled PE(O) 16:0 species concentration at 0, 1, 4, 24 and 48 timepoints for dose mix A of treatment in females. FIG. 20B shows a chart displaying labelled PE(O) 16:0 species concentration at 0, 1, 4, 24 and 48 timepoints for dose mix A of treatment in males. FIG. 20 shows mean±standard deviation (n=4-5 per group) with each symbol representing an individual sample.

FIG. 21 shows labelled phosphatidylethanolamine plasmalogen (PE(P)) concentration after dose mix A of plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 21A shows a chart displaying labelled PE(P) 16:0 species concentration at 0, 1, 4, 24 and 48 timepoints for dose mix A of treatment in females. FIG. 21B shows a chart displaying labelled PE(P) 16:0 species concentration at 0, 1, 4, 24 and 48 timepoints for dose mix A of treatment in males. FIG. 21 shows mean±standard deviation (n=4-5 per group) with each symbol representing an individual sample.

FIG. 22 shows labelled phosphatidylethanolamine plasmalogen (PE(P)) concentration after dose mix B of plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 22A shows a chart displaying labelled PE(P) 16:0 species concentration at 0, 1, 4, 24 and 48 timepoints for dose mix B of treatment in females. FIG. 22B shows a chart displaying labelled PE(P) 16:0 species concentration at 0, 1, 4, 24 and 48 timepoints for dose mix B of treatment in males. FIG. 22 shows mean±standard deviation (n=4 per group) with each symbol representing an individual sample.

FIG. 23 shows the maximum concentration of labelled phosphatidylethanolamine plasmalogen (Cₘₐₓ) after plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 23A shows a bar chart displaying Cₘₐₓ for dose mix A of treatment in females. FIG. 23B shows a bar chart displaying log(2) Cₘₐₓ for dose mix A of treatment in females. FIG. 23C shows a bar chart displaying dose mix A of treatment in males. FIG. 23D shows a bar chart displaying log(2) Cₘₐₓ dose mix A of treatment in males. FIG. 23E shows a bar chart displaying dose mix B of treatment in females. FIG. 23F shows a bar chart displaying log(2) Cₘₐₓ dose mix B of treatment in females. FIG. 23G shows a bar chart displaying dose mix B of treatment in males. FIG. 23H shows a bar chart displaying log(2) Cₘₐₓ dose mix B of treatment in males. FIG. 23 shows mean±standard deviation (n=4 per group) with each symbol representing an individual sample. One-way ANOVA with Tukey's post-hoc test was used. ns indicates p>0.05, * p≤0.05, ** p≤0.01, *** p≤0.001, **** p≤0.0001.

FIG. 24 shows area under the curve values for labelled phosphatidylethanolamine plasmalogen (AUC) after plasmalogen precursor treatment in mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 24A shows a bar chart displaying AUC for dose mix A of treatment in females. FIG. 24B shows a bar chart displaying log(2) AUC for dose mix A of treatment in females. FIG. 24C shows a bar chart displaying AUC for dose mix A of treatment in males. FIG. 24D shows a bar chart displaying log(2) AUC for dose mix A of treatment in males. FIG. 24E shows a bar chart displaying AUC for dose mix B of treatment in females. FIG. 24F shows a bar chart displaying log(2) AUC for dose mix B of treatment in females. FIG. 24G shows a bar chart displaying AUC for dose mix B of treatment in males. FIG. 24H shows a bar chart displaying log(2) AUC for dose mix B of treatment in males. FIG. 24 shows mean±standard deviation (n=4 per group) with each symbol representing an individual sample. One-way ANOVA with Tukey's post-hoc test was used. ns indicates p>0.05, * p≤0.05, ** p≤0.01, *** p≤0.001, **** p≤0.0001.

FIG. 25 shows area under the curve (AUC) for lipid classes of the plasmalogen biosynthesis pathway after dose mix A of plasmalogen precursor treatment in female mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 25A shows a bar chart displaying AUC for AKG treatment. FIG. 25B shows a bar chart displaying AUC for LPC(O) treatment. FIG. 25C shows a bar chart displaying AUC for LPE(O) treatment. FIG. 25 shows mean±standard deviation (n=4 per group) with each symbol representing an individual sample.

FIG. 26 shows area under the curve (AUC) for lipid classes of the plasmalogen biosynthesis pathway after dose mix A of plasmalogen precursor treatment in male mice. Eight-week-old C57BL/6 mice were gavaged with a single dose of lecithin (vehicle control), AKG mix (AKG (O-16:0, 18:0, 18:1)d2), LPC(O) mix (LPC(O-16:0, 18:0, 18:1)d2), LPE(O) mix (LPE(O-16:0)d5 and unlabeled LPE(O-16:0, 18:0, 18:1)). Blood was collected through tail-tip bleeding before treatment and at 1, 4, 24 and 48 hours. FIG. 26A shows a bar chart displaying AKG treatment. FIG. 26B shows a bar chart displaying LPC(O) treatment. FIG. 26C shows a bar chart displaying LPE(O) treatment. FIG. 26 shows mean±standard deviation (n=4 per group) with each symbol representing an individual sample.

FIG. 27 shows labelled phosphatidylethanolamine plasmalogen of the O-16:0 species in brain after supplementation of labelled LPE(O) in mice. Eight-week-old C57BL/6 mice were gavaged with a single dose of lecithin (vehicle control), or LPE(O) mix (LPE(O-16:0, O-18:0, O-18:1, O-16:0-d5)). Brain samples were collected 48 hours after gavage. FIG. 27A shows a bar chart displaying dose mix A treatment in females. FIG. 27B shows a bar chart displaying dose mix A treatment in males. FIG. 27C shows a bar chart displaying dose mix B of treatment in females. FIG. 27D shows a bar chart displaying dose mix B of treatment in males. FIG. 27 shows mean±standard deviation (n=4 per group), with each symbol representing an individual sample. Student's t test was used to compare the mean differences between the groups. * p≤0.05, *** p≤0.001;

FIG. 28 shows labelled phosphatidylethanolamine plasmalogen of the O-16:0 species in brain after supplementation of labelled AKG or LPC(O) in mice. Eight-week-old C57BL/6 mice were gavaged with a single dose of lecithin (vehicle control), AKG mix (AKG (O-16:0-d2, O-18:0-d2, O-18:1-d2)) or LPC(O) mix (LPC(O-16:0-d2, O-18:0-d2. Brain samples were collected 48 hours after gavage. FIG. 28A shows a bar chart displaying dose mix A of treatment in females, FIG. 28B shows a bar chart displaying dose mix A treatment in males. FIG. 28C shows a bar chart displaying dose mix B of treatment in females. FIG. 28D shows a bar chart displaying dose mix B of treatment in males. FIG. 28 shows mean±standard deviation (n=4 per group), with each symbol representing an individual sample. One-way ANOVA was used to test the mean differences between the groups.

FIG. 29 shows changes in the tracer alkyl diacylglycerol [TG(O)] concentration following administration of different precursor compounds. Precursor compounds are FIG. 29A AKG-dose mix C; FIG. 29B AKDAG-oleic-dose mix C; FIG. 29C AKDAG-DHA-dose mix C; FIG. 29D LPC(O)-dose mix C; FIG. 29E AKG-dose mix D; FIG. 29F AKDAG-oleic-dose mix E; FIG. 29G AKDAG-DHA-dose mix D; FIG. 29H LPC(O)-dose mix D.

FIG. 30 shows changes in the tracer lysoalkylphosphatidylcholine [LPC(O)] concentration following administration of different precursor compounds. Precursor compounds are FIG. 30A AKG-dose mix C; FIG. 30B AKDAG-oleic-dose mix C; FIG. 30C AKDAG-DHA-dose mix C; FIG. 30D LPC(O)-dose mix C; FIG. 30E AKG-dose mix D; FIG. 30F AKDAG-oleic-dose mix E; FIG. 30G AKDAG-DHA-dose mix D; FIG. 30H LPC(O)-dose mix D.

FIG. 31 shows changes in the tracer alkylphosphatidylcholine [PC(O)] concentration following administration of different precursor compounds. Precursor compounds are FIG. 31A AKG-dose mix C; FIG. 31B AKDAG-oleic-dose mix C; FIG. 31C AKDAG-DHA-dose mix C; FIG. 31D LPC(O)-dose mix C; FIG. 31E AKG-dose mix D; FIG. 31F AKDAG-oleic-dose mix E; FIG. 31G AKDAG-DHA-dose mix D; FIG. 31H LPC(O)-dose mix D.

FIG. 32 shows changes in the tracer alkylphosphatidylethanolamine [PE(O)] concentration following administration of different precursor compounds. Precursor compounds are FIG. 32A AKG-dose mix C; FIG. 32B AKDAG-oleic-dose mix C; FIG. 32C AKDAG-DHA-dose mix C; FIG. 32D LPC(O)-dose mix C; FIG. 32E AKG-dose mix D; FIG. 32F AKDAG-oleic-dose mix E; FIG. 32G AKDAG-DHA-dose mix D; FIG. 32H LPC(O)-dose mix D.

FIG. 33 shows changes in the tracer alkenylphosphoethanolamine [PE(P)] concentration following administration of different precursor compounds. Precursor compounds are FIG. 33A AKG-dose mix C; FIG. 33B AKDAG-oleic-dose mix C; FIG. 33C AKDAG-DHA-dose mix C; FIG. 33D LPC(O)-dose mix C; FIG. 33E AKG-dose mix D; FIG. 33F AKDAG-oleic-dose mix E; FIG. 33G AKDAG-DHA-dose mix D; FIG. 33H LPC(O)-dose mix D.

FIG. 34 shows changes in the tracer alkenylphosphatidylcholine [PC(P)] concentration following administration of different precursor compounds. Precursor compounds are FIG. 34A AKG-dose mix C; FIG. 34B AKDAG-oleic-dose mix C; FIG. 34C AKDAG-DHA-dose mix C; FIG. 34D LPC(O)-dose mix C; FIG. 34E AKG-dose mix D; FIG. 34F AKDAG-oleic-dose mix E; FIG. 34G AKDAG-DHA-dose mix D; FIG. 34H LPC(O)-dose mix D.

FIG. 35 shows Maximum plasma concentration (Cmax) of major lipid classes following administration of different precursor compounds. Precursor compounds are FIG. 35A TG(O); FIG. 35B LPC(O); FIG. 35C PC(O); FIG. 35D PE(O); FIG. 35E PE(P); FIG. 35F PC(P).

FIG. 36 shows Area under the curve (AUC) of major lipid classes following administration of precursor compounds. Precursor compounds are FIG. 36A TG(O); FIG. 36B LPC(O); FIG. 36C PC(O); FIG. 36D PE(O); FIG. 36E PE(P); FIG. 36F PC(P).

FIG. 37 shows the effect of labelled precursor supplementation on the major ether lipids classes in liver. Precursor compounds are FIG. 37A TG(O); FIG. 37B LPC(O); FIG. 37C PC(O); FIG. 37D PE(O); FIG. 37E PE(P); FIG. 37F PC(P).

FIG. 38 shows the effect of labelled precursor supplementation on the major ether lipids classes in spleen. Precursor compounds are FIG. 38A TG(O); FIG. 38B LPC(O); FIG. 38C PC(O); FIG. 38D PE(O); FIG. 38E PE(P); FIG. 38F PC(P).

FIG. 39 shows the effect of labelled precursor supplementation on the major ether lipids classes in brain. Precursor compounds are FIG. 39A TG(O); FIG. 39B LPC(O); FIG. 39C PC(O); FIG. 39D PE(O); FIG. 39E PE(P); FIG. 39F PC(P).

FIG. 40 shows the effect of labelled precursor supplementation on the major ether lipids classes in kidney. Precursor compounds are FIG. 40A TG(O); FIG. 40B LPC(O); FIG. 40C PC(O); FIG. 40D PE(O); FIG. 40E PE(P); FIG. 40F PC(P).

FIG. 41 shows the effect of labelled precursor supplementation on the major ether lipids classes in visceral adipose tissue. Precursor compounds are FIG. 41A TG(O); FIG. 41B LPC(O); FIG. 41C PC(O); FIG. 41D PE(O); FIG. 41E PE(P); FIG. 41F PC(P).

FIG. 42 shows the effect of labelled precursor supplementation on the major ether lipids classes in skeletal muscle. Precursor compounds are FIG. 42A TG(O); FIG. 42B LPC(O);

FIG. 42C PC(O); FIG. 42D PE(O); FIG. 42E PE(P); FIG. 42F PC(P).

FIG. 43 shows the effect of labelled precursor supplementation on the major ether lipids classes in heart. Precursor compounds are FIG. 43A TG(O); FIG. 43B LPC(O); FIG. 43C PC(O); FIG. 43D PE(O); FIG. 43E PE(P); FIG. 43F PC(P).

FIG. 44 shows the effect of LPC(O) supplementation on plasmalogen level in RAW 264.7 cells. RAW 264.7 macrophages cells were treated with 20 μM of LPC(O) with different SN1 compositions for 24 hours and then harvested for lipidomic analysis. The PE plasmalogen or PE(P) data are normalised to total cellular phosphatidylcholine (PC) level and presented as mean±SD (n=3/group). Each circle represents individual data point. Mean difference between the groups were compared by one-way ANOVA followed by Fishers LSD test. *indicates P<0.05, **indicates P<0.01 and **** indicates P<0.0001 vs control.

FIG. 45 shows the effect of LPE(O) supplementation on plasmalogen level in RAW 264.7 cells. RAW 264.7 macrophages cells were treated with 20 μM of LPE(O) with different SN1 compositions for 24 hours and then harvested for lipidomic analysis. The PE plasmalogen or PE(P) data are normalised to total cellular phosphatidylcholine (PC) level and presented as mean±SD (n=3/group). Each circle represents individual data point. Mean difference between the groups were compared by one-way ANOVA followed by Fishers LSD test. **** indicates P<0.0001 vs control.

FIG. 46 shows the effect of LPC(O) supplementation on plasmalogen composition in RAW 264.7 cells. RAW 264.7 macrophages cells were treated with 20 μM of LPC(O) with different SN1 compositions for 24 hours and then harvested for lipidomic analysis. The relative proportions of different SN1 containing PE(P)s are presented as mean±SD (n=3/group).

FIG. 47 shows the effect of LPC(O) supplementation on plasmalogen level in 3T3-L1 cells. 3T3-L1 pre-adipocyte cells were treated with 20 μM of LPC(O) with different SN1 compositions for 24 hours and then harvested for lipidomic analysis. The PE plasmalogen or PE(P) data are normalised to total cellular phosphatidylcholine (PC) level and presented as mean±SD (n=3/group). Each circle represents individual data point. Mean difference between the groups were compared by one-way ANOVA followed by Fishers LSD test. *indicates P<0.05, **indicates P<0.01 and **** indicates P<0.0001 vs control.

FIG. 48 shows the effects of LPE(O) supplementation on plasmalogen level in 3T3-L1 cells. 3T3-L1 pre-adipocyte cells were treated with 20 μM of LPE(O) with different SN1 compositions for 24 hours and then harvested for lipidomic analysis. The PE plasmalogen or PE(P) data are normalised to total cellular phosphatidylcholine (PC) level and are presented as mean±SD (n=3/group). Each circle represents individual data point. Mean difference between the groups were compared by one-way ANOVA followed by Fishers LSD test. *indicates P<0.05, **indicates P<0.01, ***indicates P<0.001 and **** indicates P<0.0001 vs control.

FIG. 49 shows the effects of LPC(O) supplementation on plasmalogen composition in 3T3-L1 cells. 3T3-L1 cells were treated with 20 μM of LPC(O) with different SN1 compositions for 24 hours and then harvested for lipidomic analysis. The relative proportions of different SN1 containing PE(P) are presented as mean±SD (n=3/group).

FIG. 50 shows the effects of LPE(O) supplementation on plasmalogen composition in 3T3-L1 cells. 3T3-L1 cells were treated with 20 μM of LPE(O) with different SN1 compositions for 24 hours and then harvested for lipidomic analysis. The relative proportions of different SN1 containing PE(P) are presented as mean±SD (n=3/group).

FIG. 51 shows the label incorporation (Area under the curve) into lipid classes of the plasmalogen biosynthesis pathway after low dose of plasmalogen precursor supplementation. FIG. 51A shows incorporation following AKG supplementation; FIG. 51B shows incorporation following AKDAG-oleic supplementation; FIG. 51C shows incorporation following AKDAG-DHA supplementation; FIG. 51D shows incorporation following LPC(O) supplementation.

FIG. 52 shows the % distribution of label incorporation in the plasmalogen biosynthesis pathway after plasmalogen precursor supplementation. FIG. 52A shows incorporation following AKG supplementation; FIG. 52B shows incorporation following AKDAG-oleic supplementation; FIG. 52C shows incorporation following AKDAG-DHA supplementation; FIG. 52D shows incorporation following LPC(O) supplementation.

FIG. 53 shows the labelled ether lipids in feces after 24 hours of labelled precursor supplementation FIG. 53A TG(O); FIG. 53B LPC(O); FIG. 53C PC(O); FIG. 53D PE(O); FIG. 53E PE(P); FIG. 53F PC(P).

FIG. 54 shows the labelled ether lipids in feces after 24 hours of labelled precursor supplementation FIG. 54A TG(O); FIG. 54B LPC(O); FIG. 54C PC(O); FIG. 54D PE(O); FIG. 54E PE(P); FIG. 54F PC(P).

FIG. 55 shows all of the LPX(O) treatments suppressed the LPS-induced increase in TLR4 gene expression. Data are normalized to control+LPS. Data are presented as mean SD (n=3/group). Mean difference between the groups were compared by one-way ANOVA followed by Fishers LSD test. An asterisk (*) indicates P<0.05 vs control, \hat{ } indicates P<0.05 vs control+LPS.

FIG. 56 shows all of the LPX(O) treatments suppressed the LPS-induced increase in TLR4 gene expression. Data are normalized to control. Data are presented as mean±SD (n=3/group). Mean difference between the groups were compared by one-way ANOVA followed by Fishers LSD test. *indicates P<0.05 vs control, \hat{ } indicates P<0.05 vs control+LPS.

Table 1. The list of abbreviations used in this disclosure.

TABLE 1
The list of abbreviations used in this disclosure.
Table 1

| | |
|---|---|
| 20:4 | Arachidonic acid |
| 22:6 or DHA | Docosahexaenoic acid |
| AG or AKG | Alkylglycerol |
| AKDAG or TG(O) | Alkyldiacylglycerol |
| AUC | Area under the curve |
| Cₘₐₓ | Maximum serum concentration that e.g., |
| | a drug molecule achieves after administration |
| DG | Diacylglycerol |
| DG(O) | Alkylacylglycerol |
| DPA | Docosapentaenoic acid |
| EPA | Eicosapentaenoic acid or icosapentaenoic acid |
| FA | Fatty acid |
| Far-1 | Fatty acyl-CoA reductase 1 |
| Far-2 | Fatty acyl-CoA reductase 2 |
| FFQ | Food frequency questionnaire |
| FO | Fish oil |
| KO | Krill oil |
| LC-PUFA | Long chain polyunsaturated fatty acids |
| LPA(O) | Lysoalkylphosphatidic acid |
| LPC(O) | Lysoalkylphosphatidylcholines |
| LPC(O-16:0) | Compound of Formula (I-A3) |
| LPC(O-18:0) | Compound of Formula (I-A2) |
| LPC(O-18:1) | Compound of Formula (I-A1) |
| LPE(O) | Lysoalkylphosphatidylethanolamine |
| LPE(O-16:0) | Compound of Formula (I-A6) |
| LPE(O-18:0) | Compound of Formula (I-A5) |
| LPE(O-18:1) | Compound of Formula (I-A4) |
| PC or PC(P) | Alkenylphosphatidylcholine |
| PC(O) | Alkylphosphatidylcholine |
| PE or PE(P) | Alkenylphosphatidylethanolamine |
| PE(O) | Alkylphosphatidylethanolamine |
| PUFA | Polyunsaturated fatty acids |
| SLO | Shark liver oil |
| TG | Triacylglycerol |

### DETAILED DESCRIPTION

The general structure of the phospholipids described herein is depicted by the following Formula (I):

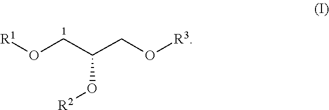

In compounds of Formula (I), the sn-1 carbon is labeled as above. R³ includes phosphate and substituted phosphate. R¹ and R² can each be independently hydrogen, alkyl, alkenyl, acyl or the like, or substituted versions of these moieties as described herein.

In Scheme I, some typical plasmalogens are shown in the top two structures, the boxes highlighting the sn-1 substitution as derived from Formula (I).

### Definitions

Unless defined otherwise, all technical and scientific terms used herein have the same meaning as commonly understood by those of ordinary skill in the art to which the disclosure belongs.

The term “and/or”, e.g., “X and/or Y” shall be understood to mean “X and Y” or “X or Y” and shall be taken to provide explicit support for both meanings or for either meaning.

The terms “a” and “an” as used herein mean “one or more” and include the plural unless the context is inappropriate.

The term “alkyl” as used herein refers to a saturated straight or branched hydrocarbon, such as a straight or branched group of 1-30, 1-18, 14-24, 14-18, 16 or 18 carbon atoms, referred to herein as C₁-C₃₀alkyl, C₁-C₁₈alkyl, C₁₄-C₂₄alkyl C₁₄-C₁₈alkyl, C₁₆alkyl, or C₁₈alkyl respectively. Exemplary alkyl groups include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, isobutyl, t-butyl, isopentyl, neopentyl, etc.

The term “alkenyl” as used herein refers to an unsaturated straight or branched hydrocarbon, such as a straight or branched group of 2-30, 2-18, 14-24, 14-18, 16 or 18 carbon atoms, referred to herein as C₂-C₃₀alkenyl, C₂-C₁₈alkenyl, C₁₄-C₂₄alkenyl C₁₄-C₁₈alkenyl, C₁₆alkenyl, or C₁₈alkenyl respectively. Multiple double bonds may be present in an alkenyl compound. Exemplary alkenyl groups include, but are not limited to, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, isopropenyl, 2-methyl-1-butenyl, 3-methyl-1-butenyl, 2-methyl-3-butenyl, 2,2-dimethyl-1-propenyl, 2-methyl-1-pentenyl, 3-methyl-1-pentenyl, 4-methyl-1-pentenyl, 2-methyl-2-pentenyl, 3-methyl-2-pentenyl, 4-methyl-2-pentenyl, 2,2-dimethyl-1-butenyl, 3,3-dimethyl-1-butenyl, 2-ethyl-1-butenyl, isobutenyl, t-butenyl, isopentenyl, neopentenyl, etc. The term “alkenyl” can additionally be taken to mean an alkene of structure C18:1 wherein the double bond is typically at the n7 or n9 position. The term “alkenyl” can additionally be taken to mean an alkene of structure C18:1 wherein the double bond is between the first and second carbon adjacent to the ether link. The term “alkenyl” can additionally be taken to mean an alkene wherein one double bond is at the n7 or n9 position and a second double bond is between the first and second carbon.

The term “acyl” as used herein refers to a radical of general formula —C(O)R, wherein R is hydrogen or a saturated or unsaturated straight or branched hydrocarbon, such as a straight or branched group of 1-30, 1-18, 14-24, 14-18, 16 or 18 carbon atoms, referred to herein as C₁-C₃₀alkyl, C₁-C₁₈alkyl, C₁₄-C₂₄alkyl C₁₄-C₁₈alkyl, C₁₆alkyl, or C₁₈alkyl respectively or a straight or branched group of 2-30, 2-18, 14-24, 14-18, 16 or 18 carbon atoms, referred to herein as C₂-C₃₀alkenyl, C₂-C₁₈alkenyl, C₁₄-C₂₄alkenyl C₁₄-C₁₈alkenyl, C₁₆alkenyl, or C₁₈alkenyl respectively. Exemplary alkyl groups include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, isobutyl, t-butyl, isopentyl, neopentyl, etc.

The term “ether” as used herein, refers to any organic compound with a structure similar to an ether functional group, having an oxygen atom linking two alkyl or other organic groups. As used herein, the term “ether” may refer to a functional group, having an oxygen atom linking a saturated straight or branched hydrocarbon, such as a straight or branched group of 1-30, 1-18, 14-24, 14-18, 16 or 18 carbon atoms, referred to herein as C₁-C₃₀alkyl, C₁-C₁₈alkyl, C₁₄-C₂₄alkyl C₁₄-C₁₈alkyl, C₁₆alkyl, or C₁₈alkyl respectively and a second saturated straight or branched hydrocarbon. As used herein, the term “ether” may also refer to a functional group, having an oxygen atom linking a saturated straight or branched hydrocarbon, such as a straight or branched group of 1-30, 1-18, 14-24, 14-18, 16 or 18 carbon atoms, referred to herein as C₁-C₃₀alkyl, C₁-C₁₈alkyl, C₁₄-C₂₄alkyl C₁₄-C₁₈alkyl, C₁₆alkyl, or C₁₈alkyl respectively and an unsaturated straight or branched hydrocarbon such as a straight or branched group of 2-30, 2-18, 14-24, 14-18, 16 or 18 carbon atoms, referred to herein as C₂-C₃₀alkenyl, C₂-C₁₈alkenyl, C₁₄-C₂₄alkenyl C₁₄-C₁₈alkenyl, C₁₆alkenyl, or C₁₈alkenyl respectively. As used herein, the term “ether” may also refer to a functional group, having an oxygen atom linking an unsaturated straight or branched hydrocarbon, such as a straight or branched group of 2-30, 2-18, 14-24, 14-18, 16 or 18 carbon atoms, referred to herein as C₂-C₃₀alkenyl, C₂-C₁₈alkenyl, C₁₄-C₂₄alkenyl C₁₄-C₁₈alkenyl, C₁₆alkenyl, or C₁₈alkenyl respectively, and a second unsaturated straight or branched hydrocarbon.

As used herein, the term “vinyl ether” refers to a group wherein an alkene is adjacent to an ether linkage.

The term “ester” as used herein, refers to any organic compound with a structure similar to an ester functional group. As referred to herein, the term “ester” refers to any compound or functional group that can be represented by a radical of general formula —RCOOR′, wherein R is hydrogen or a saturated or unsaturated straight or branched hydrocarbon, such as a straight or branched group of 1-30, 1-18, 14-24, 14-18, 16 or 18 carbon atoms, referred to herein as C₁-C₃₀alkyl, C₁-C₁₈alkyl, C₁₄-C₂₄alkyl C₁₄-C₁₅alkyl, C₁₆alkyl, or C₁₈alkyl respectively or a straight or branched group of 2-30, 2-18, 14-24, 14-18, 16 or 18 carbon atoms, referred to herein as C₂-C₃₀alkenyl, C₂-C₁₈alkenyl, C₁₄-C₂₄alkenyl C₁₄-C₁₈alkenyl, C₁₆alkenyl, or C₁₈alkenyl respectively, and R′ is a saturated or unsaturated straight or branched hydrocarbon, such as a straight or branched group of 1-30, 1-18, 14-24, 14-18, 16 or 18 carbon atoms, referred to herein as C₁-C₃₀alkyl, C₁-C₁₈alkyl, C₁₄-C₂₄alkyl C₁₄-C₁₈alkyl, C₁₆alkyl, or C₁₈alkyl respectively or a straight or branched group of 2-30, 2-18, 14-24, 14-18, 16 or 18 carbon atoms, referred to herein as C₂-C₃₀alkenyl, C₂-C₁₈alkenyl, C₁₄-C₂₄alkenyl C₁₄-C₁₈alkenyl, C₁₆alkenyl, or C₁₈alkenyl respectively.

The terms “substituted” and “optionally substituted” in reference to phosphate groups, alkyl groups, alkenyl groups, or acyl groups refers to the optional substitution of these groups by an additional moiety. In each case, the substituent or additional moiety may be independently selected from hydrogen, C₁₋₃alkyl, and halogen (e.g., Cl, F, Br or I). In certain embodiments, the group is not substituted, i.e., it is unsubstituted.

The naming convention for lipids used here follows the guidelines established by the Lipid Maps Consortium and the shorthand notation of Liebisch et al. (Liebisch et al., Fahy et. al. (2009), Fahy et al. (2005)]. Alternatively, the nomenclature is based on Biochemical Nomenclature and Related Documents, 2nd edition, Portland Press (London, 1992) Edited C Liébecq. [ISBN 1-85578-005-4], “Nomenclature of Lipids.” Phospholipids typically contain two fatty acid chains and in the absence of detailed characterization are expressed as the sum composition of carbon atoms and double bonds (e.g. PC(38:6)). However, where an acyl chain composition has been determined the naming convention indicates this (e.g. PC(38:6) is changed to PC(16:0/22:6)).

The present disclosure refers to lipid molecules using the numbering system X:Y. The number X represents the number of carbon atoms present in the chain.

In the context of alkylglycerols, alkylacylglycerols, and alkyldiacylglycerols, the number Y represents the number of double bonds present in the chain. For example, an alkylglycerol numbered as 16:0 contains a hydrocarbon group having a 16-carbon chain with no double bonds. As a further example, an alkylglycerol numbered as 18:1 contains a hydrocarbon group having an 18-carbon chain with 1 double bond.

In the context of plasmalogens/plasmenyl phospholipids, the number Y in the first listed alkenyl chain (i.e. PE(P-X:Y/X:Y) represents the number of double bonds present in the alkenyl chain in addition to the vinyl ether group. For example, in a plasmalogen numbered as PE(P-16:0/20:4) the 16:0 alkenyl group contains a hydrocarbon group having a 16-carbon chain with no double bonds other than the vinyl ether group (i.e., there is a double bond between the first 2 carbons and the remaining 14 carbons are saturated). As another example, in a plasmalogen numbered as PE(P-18:1/20:4), the 18:1 alkenyl group contains a hydrocarbon group having an 18-carbon chain with 1 double bond in addition to the vinyl ether group (i.e., there is a double bond between the first 2 carbon atoms, and there is one other double bond between 2 carbons out of the remaining 16 carbons). As shown above in Scheme I, the plasmenyl PE plasmalogen is represented by the structure PE(P-16:0/22:6).

Where ether lipids contain one or more double bonds, the double bonds may be located at various positions in the hydrocarbon chains. For example, an alkylglycerol numbered as 18:1 may contain a mixture of species, e.g. with cis-n1 and cis-n9 double bonds. As another example, a plasmalogen (e.g., PE(P)) numbered as 18:1 may contain a mixture of species, e.g., with cis-n1 and cis-n9 double bonds.

As used herein, the term “plasmanyl” shall be understood to refer to phospholipids having an ether bond in the sn-1 position to an alkyl group.

As used herein, the term “plasmenyl” shall be understood to refer to phospholipids having an ether bond in the sn-1 position to an alkenyl group. The plasmenyl phospholipids are referred to as “plasmalogens”.

A plasmalogen having a “15:0” alkenyl group is a molecule having an ether bond in the sn-1 position to a 15-carbon chain which contains a double bond between carbons 1 and 2 (typically a cis-vinyl ether group), and no other double bonds in the chain.

A plasmalogen having a “16:0” alkenyl group is a molecule having an ether bond in the sn-1 position to a 16-carbon chain which contains a double bond between carbons 1 and 2 (typically a cis-vinyl ether group), and no other double bonds in the chain.

A plasmalogen having a “17:0” alkenyl group is a molecule having an ether bond in the sn-1 position to a 17-carbon chain which contains a double bond between carbons 1 and 2 (typically a cis-vinyl ether group), and no other double bonds in the chain.

A plasmalogen having an “18:0” alkenyl group is a molecule having an ether bond in the sn-1 position to an 18-carbon chain which contains a double bond between carbons 1 and 2 (typically a cis-vinyl ether group), and no other double bonds in the chain.

A plasmalogen having an “18:1” alkenyl group is a molecule having an ether bond in the sn-1 position to an 18-carbon chain which contains a double bond between carbons 1 and 2 (typically a cis-vinyl ether group), and having one additional double bond, typically between carbons 7 and 8 (e.g., n7), between carbons 9 and 10 (e.g., n9), or between carbons 11 and 12 (e.g., n 11), and typically a cis-double bond.

A plasmalogen having a “20:0” alkenyl group is a molecule having an ether bond in the sn-1 position to a 20-carbon chain which contains a double bond between carbons 1 and 2 (typically a cis-vinyl ether group), and no other double bonds in the chain.

A plasmalogen having an “20:1” alkenyl group is a molecule having an ether bond in the sn-1 position to a 20-carbon chain which contains a double bond between carbons 1 and 2 (typically a cis-vinyl ether group), and having one additional double bond, typically between carbons 7 and 8 or between carbons 9 and 10, and typically a cis-double bond.

A plasmalogen having an “18:2” acyl alkenyl group is a molecule having an ester bond in the sn-2 position to an 18-carbon chain which has two double bonds, typically between carbons 9 and 10, and between carbons 11 and 12, and typically cis-double bonds.

A plasmalogen having a “20:4” acyl alkenyl group is a molecule having an ester bond in the sn-2 position to a 20-carbon chain which has four double bonds, typically between carbons 5 and 6, carbons 8 and 9, carbons 11 and 12, and carbons 14 and 15, and typically cis-double bonds.

A plasmalogen having a “22:6” acyl alkenyl group is a molecule having an ester bond in the sn-2 position to a 22-carbon chain which has six double bonds, typically between carbons 4 and 5, carbons 7 and 8, carbons 10 and 11, carbons 13 and 14, carbons 16 and 17, and carbons 19 and 20, and typically cis-double bonds.

As used herein, “acyl alkenyl” refers to a straight or branched chain hydrocarbon containing, for example, from 2 to 30 carbons and containing at least one carbon-carbon double bond, which is covalently bonded to an acyl group. For example, the use of nomenclature such as 22:6 or 18:2 in the context of an acyl alkenyl group refers to an acyl alkenyl group having 22 carbons or 18 carbons respectively, and having 6 or 2 double bonds respectively. An example of an acyl alkenyl group is:

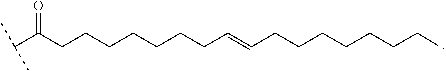

Acyl alkenyl groups may be present in species such as alkylacylglycerols or alkyldiacylglycerols (as an acyl group), or as an acyl group in plasmanyl- or plasmenyl-phospholipids. Typically, when present in those species, there is no double bond between the carbons which are α- and β- to the acyl group.

As used herein, “acyl alkyl” refers to a straight or branched chain hydrocarbon containing, for example, from 1 to 30 carbons, which is covalently bonded to an acyl group. For example, the use of nomenclature 22:0 or 18:0 in the context of an acyl alkyl group refers to an acyl alkyl group having 22 carbons or 18 carbons respectively. An example of an acyl alkyl group is:

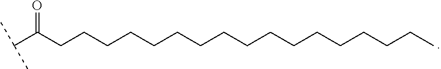

It will also be recognized that the compounds described herein may possess asymmetric centers and are therefore capable of existing in more than one stereoisomeric form. The disclosure thus also relates to compounds that, in at least some embodiments, are in substantially pure isomeric form at one or more asymmetric centers e.g., that have an enantiopurity that is equal to or greater than 90% enantiomeric excess (“ee”), such as 95% ee, 97% ee, 99% ee, or greater than 99% ee. The disclosure also relates to compounds present as mixtures of stereoisomeric forms, including racemic, diastereomeric, or scalemic mixtures. Such isomers may be naturally occurring or may be prepared by asymmetric synthesis, including but not limited to, synthetic methods employing chiral intermediates, or by chiral resolution. Certain compounds contained in compositions of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included. If, for instance, a particular enantiomer of a compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The present disclosure also relates to derivatives of glycerol. Whilst glycerol is achiral, derivatives are typically chiral. Typically, the glycerol utilized will have a stereochemical configuration corresponding to that found in nature. In some embodiments, the glycerol derivatives utilized have the stereochemical configuration as shown above in Formula (I).

As referred to herein, the term “alkylglycerol” means a compound of Formula (I) in which the R¹ group is a hydrocarbon chain and the R² and R³ groups are each hydrogen. The term “alkylglycerol” is additionally taken to mean a compound of Formula (I-A), wherein R_{A} is a hydrocarbon chain, and the Rₓ groups are each independently selected from hydrogen or C₁₋₃alkyl. The term “alkylglycerol” is additionally taken to mean a compound of Formula (I-A1), (I-A2) and (I-A3). Although the term “alkylglycerol” is used, it will be understood by those of skill in the art that the term encompasses species with hydrocarbon groups at the R¹ position that include unsaturation in the hydrocarbon chain. However, as used herein, an “alkylglycerol” compound does not contain a double bond between carbons 1 and 2 of the hydrocarbon chain, i.e., proximal to the ether linkage.

An alkylglycerol having a “16:0” group is a molecule having an ether bond in the sn-1 position to a 16-carbon saturated hydrocarbon chain, and no double bonds in the chain, which may also be known as chimyl alcohol.

An alkylglycerol having an “18:0” group is a molecule having an ether bond in the sn-1 position to an 18-carbon saturated hydrocarbon chain, and no double bonds in the chain, which may also be known as batyl alcohol.

An alkylglycerol having an “18:1” group is a molecule having an ether bond in the sn-1 position to an 18-carbon hydrocarbon chain, which contains one double bond, typically between carbons 9 and 10 and typically a cis-double bond, which may also be known as selachyl alcohol.

As referred to herein, the term “alkylacylglycerol” means a compound of Formula (I) in which the R¹ group is a hydrocarbon chain, one of the R² and R³ groups is hydrogen, and the other of the R² and R³ groups is an acyl group, individual selected from an acyl alkyl group and an acyl alkenyl group. Although the term “alkylacylglycerol” is used, it will be understood by those of skill in the art that the term encompasses species with hydrocarbon groups at the R¹ position that include unsaturation in the hydrocarbon chain. However, as used herein, an “alkylacylglycerol” does not contain a double bond between carbons 1 and 2 of the R¹ hydrocarbon chain, i.e., proximal to the ether linkage.

As referred to herein, the term “alkyldiacylglycerol” means a compound of Formula (I) in which the R¹ group is a hydrocarbon chain, and the R² and R³ groups are acyl groups individually selected from acyl alkyl and an acyl alkenyl. Although the term “alkyldiacylglycerol” is used, it will be understood by those of skill in the art that the term encompasses species with hydrocarbon groups at the R¹ position which include unsaturation in the hydrocarbon chain. However, as used herein, an “alkyldiacylglycerol” does not contain a double bond between carbons 1 and 2 of the R¹ hydrocarbon chain, i.e., proximal to the ether linkage.

As used herein, the term “LPX(O)” refers to an LPC(O) or an LPE(O) as used elsewhere herein. In some embodiments, the LPX(O) is selected from one or more of LysoPAF-C and LysoPAF-O. In some embodiments, LysoPAF-C is selected from one or more of LysoPAF-C16, LysoPAF-C18, and LysoPAF-C18:1.

The term “fatty acid” may refer to, for example, stearic acid, palmitic acid, or oleic acid.

As used herein, the terms “subject” and “patient” refer to humans or animals to be treated by the methods of the present disclosure. In one embodiment, the subject is a human. In one embodiment, the subject is an animal such as a domestic animal, a working animal or a farm animal. Domestic animals include, but are not limited to, rabbits, birds, cats, dogs, fishes, rats, tortoises, reptiles (lizard, snake) and the like. Working animals include, but are not limited to, cattle, yaks, and horses and the like. Farm animals include, but are not limited to sheep, pigs, cows, chickens, goats, geese, ducks, llamas and the like. In some embodiments, it is envisaged that the compositions described herein will be formulated as an animal feed or dietary supplement for animals. In some embodiments, administration may be oral administration. In some embodiments, administration may be via any appropriate route including a liquid form, a capsule, a tablet, or a lozenge.

The term “non-disease state” as used herein, refers to a state in which the subject is not suffering from a plasmalogen related disease or deficiency requiring treatment.

As used herein, the term “treating” includes any effect, e.g., lessening, reducing, increasing, maintaining, modulating, ameliorating or eliminating, that results in the improvement of the condition, disease, disorder, and the like, or ameliorating a symptom thereof.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route.

As used herein, the term “composition” refers to a product comprising a particular ingredient in a particular amount and any product directly or indirectly brought about by the combination of particular ingredients in particular amounts. In some embodiments, a composition comprises an active ingredient and an inactive ingredient. In some embodiments, the composition is a formulation. In some embodiments, the formulation is a composition that is suitable for administration to and/or consumption by a subject, such as a human. A composition may be a pharmaceutical composition, and a formulation may be a pharmaceutical formulation. In some embodiments, the pharmaceutical composition or pharmaceutical formulation comprises a combination of an active agent with a pharmaceutically acceptable carrier, diluent, excipient, solubilizing agent, or vehicle, inert or active. As used herein, the term “pharmaceutically acceptable” means that a carrier, diluent, excipient, solubilizing agent, or vehicle is compatible with other components of a formulation and is nontoxic to a subject. Thus, it is to be understood that a “pharmaceutical composition” or a “pharmaceutical formulation” is appropriate for administration to and/or consumption by a subject, such as a human, and may, for example, be approved by the U.S. Food and Drug Administration and/or the European Medicines Agency for such administration and/or consumption. It is also to be understood that a composition or formulation may not necessarily be, for example, approved by the U.S. Food and Drug Administration and/or the European Medicines Agency for administration to and/or consumption by a subject, such as a human.

Pharmaceutical compositions include a product comprising an active ingredient and an inert ingredient constituting a carrier and include every product directly or indirectly brought about by the combination, complexation or aggregation of any two or more ingredients or the dissociation, other kinds of reactions or interaction of one or more ingredients. Thus, the pharmaceutical composition of the present disclosure includes every composition prepared by mixing the at least one compound of the present disclosure with a pharmaceutically acceptable carrier, diluent, excipient, solubilizing agent, or vehicle.

In some embodiments, a formulation of the disclosure is in the form of a beverage or a food product. In some embodiments, the beverage or food product is formulated for general consumption, such as by being food grade. In some embodiments, the beverage or food product is formulated as a dietary supplement or other nutritional composition. In some embodiments, the beverage or food product is pharmaceutical grade. For the avoidance of doubt, it is to be understood that, in some embodiments, a formulation of the disclosure may be suitable for consumption by a subject, such as a human, but not necessarily be of pharmaceutical grade (for example, by being of food grade or nutritional supplement grade, but not necessarily pharmaceutical grade).

In some embodiments, the formulation may contain one or more of a solubilizing agent, an emulsifier, a stabilizer, a dispersal agent, an anti-foaming agent, or a diluent. The formulation may additionally contain one or more antioxidant compounds. It is anticipated that any antioxidants suitable for oral administration, such as vitamin A, vitamin E, vitamin C, retinal, tocopherol, L-ascorbyl palmitate, riboflavin and carotenoids, including lutein, beta-carotene, zeaxanthin, and lycopene, and combinations thereof, may be formulated into an embodiment described herein. In some embodiments, the composition is naturally unreactive to oxidation and does not require the addition of antioxidant compounds.

As used herein, the term “excipient” shall mean an inactive ingredient used as a vehicle (e.g., water, capsule shell, etc.), a diluent, or a component to constitute a dosage form or pharmaceutical composition comprising a drug such as a therapeutic agent. The term also encompasses an inactive ingredient that imparts cohesive function (e.g., binder), disintegrating function (e.g., disintegrator), lubricant function (e.g., lubricating agent), and/or the other function (e.g., solvent, surfactant, etc.) to the composition.

As used herein, the term “pharmaceutically acceptable salt” refers to any pharmaceutically acceptable salt (e.g., acid or base) of a compound of the present disclosure which, upon administration to a subject, is capable of providing a compound of this disclosure or an active metabolite or residue thereof. For example, Berge et al. describe pharmaceutically acceptable salts in detail in J. Pharmaceutical Sciences (1977) 66:1-19. As is known to those of skill in the art, “salts” of the compounds of the present disclosure may be derived from inorganic or organic acids and bases. The salts can be prepared in situ during the final isolation and purification of the compounds of the disclosure or separately by reacting a free base function with a suitable acid. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids while not in themselves pharmaceutically acceptable, may be employed in the preparation of salts useful as intermediates in obtaining the compounds of the disclosure and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW₄⁺, wherein each W is independently selected from H or C₁₋₄alkyl, and the like. Basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain alkyl halides such as decyl, lauryl, myristyl, and stearyl chlorides, bromides, and iodides; arylalkyl halides such as benzyl and phenethyl bromides; and others. Products having modified solubility or dispersibility are thereby obtained.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH₄⁺, and NW₄⁺ (wherein each W is independently selected from H or C₁₋₄alkyl), and the like. For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see REMINGTON'S PHARMACEUTICAL SCIENCES (Mack Publishing Co., Easton, PA).

As used herein, in some embodiments, the term “carrier” refers to a pharmaceutically acceptable carrier. In some embodiments, the term “carrier” refers to any suitable carrier, which may not be of pharmaceutical grade. For example, in some embodiments, the carrier is of food grade but may not be of pharmaceutical grade.

The term “day” as used herein is taken to mean a 24 hour period of time.

As a general matter, compositions specifying a percentage are by weight unless otherwise specified.

### Ether Lipids and Compositions

The present disclosure shows that ether lipids such as plasmanyl-phospholipids and plasmenyl-phospholipids (plasmalogens) have in vivo profiles which are associated with healthy state, and which are associated with conditions such as diabetes. It has also been found that the in vivo ether lipid profile can be affected by the administration of compositions containing ether lipids to subjects, e.g., by supplementation with krill oil.

The formulations of the present disclosure are useful in maintaining and/or modifying in vivo ether lipid levels at levels and/or ratios associated with a natural, non-disease state (that is, the natural state of a healthy human subject), and/or in modifying or modulating in vivo ether lipid levels towards levels and/or ratios which are associated with a natural, non disease state (that is, the natural state of a healthy human subject). An exemplary, but non-limiting example of the term “healthy human subject” is taken to mean a subject or patient who is not suffering from a disease or disorder associated with a plasmalogen deficiency.

Examples of compounds useful in maintaining and/or modifying in vivo ether lipid levels and/or ratios thereof include alkyl glycerols, alkyl acyl glycerols (a compound which is an ether derivable from a glycerol alcohol and an alkyl alcohol, and which has an acyl group derivable from another glycerol alcohol and an acid), alkyl diacyl glycerols, and ether phospholipids, including but not limited to LPC(O)s, LPE(O)s, LPA(O)s, plasmanyl-phospholipids and plasmenyl-phospholipids. In some embodiments, the formulation is for in vivo maintenance and/or in vivo modification of plasmanyl- and/or plasmenyl-phospholipid levels and/or ratios thereof. In some embodiments, the plasmanyl- and/or plasmenyl-phospholipids include those having phosphatidylcholine and/or phosphatidylethanolamine groups. In some embodiments, the formulation is for in vivo maintenance and/or in vivo modification of plasmenyl-phospholipid (plasmalogen) levels and/or ratios thereof. In some embodiments, the formulation is for in vivo maintenance and/or modification of phosphatidylethanolamine plasmenyl-phospholipid (plasmalogen) levels and/or ratios thereof.

Table 2. Examples of plasmanyl-phospholipids, plasmenyl-phospholipids, and related lipidic species, and their abbreviations.

TABLE 2

| Lipid Description | Abbrev. |
|---|---|
| Alkenylphosphatidylcholine = plasmalogen | PC(P) |
| Alkenylphosphatidylethanolamine = plasmalogen | PE(P) |
| Alkylphosphatidylcholine | PC(O) |
| Alkylphosphatidylethanolamine | PE(O) |
| Lysoalkylphosphatidylcholine | LPC(O) |
| Lysoalkylphosphatidylethanolamine | LPE(O) |
| Lysoalkylphosphatidic acid | LPA(O) |
| Alkylglycerol | AG, or AKG |
| Diacylglycerol | DG |
| Triacylglycerol | TG |
| Alkyl-diacylglycerol | TG(O), or AKDAG |
| Alkyl-acylglycerol | DG(O) |
| Fatty acid(s) | FAs |

As described herein healthy subjects tend to have a plasmanyl-phospholipid and plasmenyl-phospholipid profile in which certain alkenyl ether and alkyl ether groups are present. For example, a high proportion of ether lipids (e.g., plasmanyl- and/or plasmenyl-phospholipids inclusive of plasmalogens) having 18:1 alkenyl ether groups, 18:0 alkyl ether groups and 16:0 alkyl ether groups were found in the group of healthy subjects. It is noted that the recited alkyl/alkenyl groups also contain a double bond between carbons 1 and 2 (a cis-vinyl ether group), as described above for several plasmalogen exemplary compounds.

In some embodiments, the formulation is for in vivo maintenance and/or modification of ether lipids (e.g., plasmanyl- and/or plasmenyl-phospholipids) having 18:1 alkenyl ether groups, 18:0 alkyl ether groups and 16:0 alkyl ether groups. In some embodiments, the formulation is for in vivo maintenance and/or modification of levels of plasmalogens having 18:1 ether groups, 18:0 ether groups and 16:0 ether groups. In some embodiments, the formulation is for in vivo maintenance of ether lipids (e.g., plasmanyl- and/or plasmenyl-phospholipids) at, or for in vivo modification of ether lipids (e.g., plasmanyl- and/or plasmenyl-phospholipids) towards, an in vivo total ether lipid (e.g., plasmanyl- and/or plasmenyl-phospholipid) profile in which the ether lipids (e.g., plasmanyl- and/or plasmenyl-phospholipids) have a molar ratio of 18:1 alkenyl ether groups to 18:0 alkyl ether groups to 16:0 alkyl ether groups of about 1:1.7:1.4. In some embodiments, the formulation is for in vivo maintenance of ether lipids (e.g., plasmanyl- and/or plasmenyl-phospholipids) at, or for in vivo modification of ether lipids (e.g., plasmanyl- and/or plasmenyl-phospholipids) towards, an in vivo total ether lipid (e.g., plasmanyl- and/or plasmenyl-phospholipid) profile in which the ether lipids (e.g., plasmanyl- and/or plasmenyl-phospholipids) have a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%; or having a molar percent of 18:1 alkenyl ether groups of about 23.3%, a molar percent of 18:0 alkyl ether groups of about 39.2%, and a molar percent of 16:0 alkyl ether groups of about 32.1%.

In some embodiments, the formulation is for in vivo maintenance of ether lipids at, or for in vivo modification of ether lipids towards, an in vivo plasmalogen lipid profile in which the ether lipids have a molar ratio of 18:1 ether groups to 18:0 ether groups to 16:0 ether groups of about 1:1.7:1.4. In some embodiments, the formulation is for in vivo maintenance of ether lipids at, or for in vivo modification of ether lipids towards, an in vivo plasmalogen lipid profile in which the ether lipids have a molar percent of 18:1 ether groups in the range of from 18.6% to 27.9%, a molar percent of 18:0 ether groups in the range of from 32.6% to 45.8%, and a molar percent of 16:0 ether groups in the range of from 26.8% to 37.4%; or having a molar percent of 18:1 ether groups of about 23.3%, a molar percent of 18:0 ether groups of about 39.2%, and a molar percent of 16:0 ether groups of about 32.1%.

In some embodiments, the composition comprises at least one isolated compound. In some embodiments, the at least one isolated compound is found at a purity of greater than 99%. In a preferred embodiment, the at least one isolated compound is found at a purity of greater than 99.9%.

In some embodiments the composition comprises a mixture of at least two compounds wherein the % (w/v) of one compound is at least 90%.

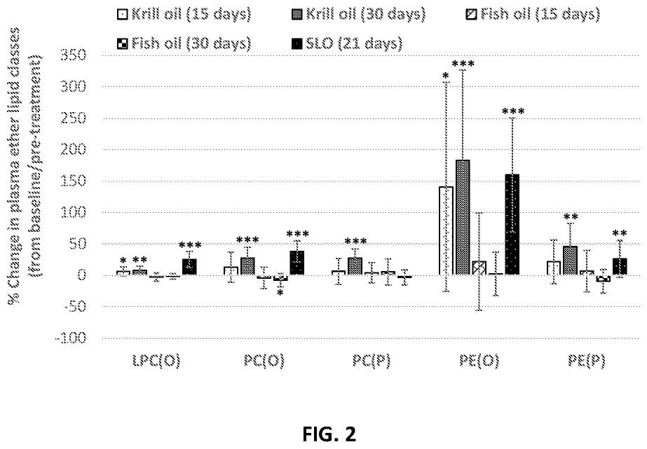

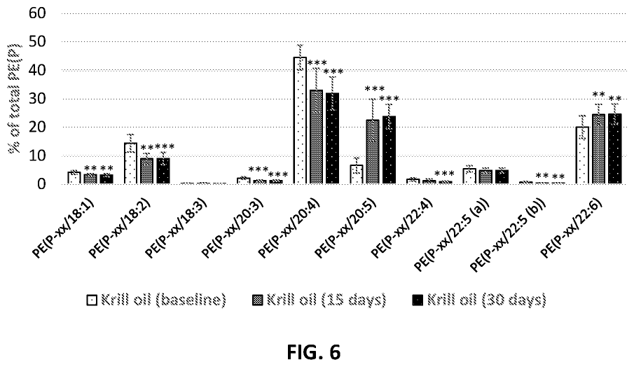

In one aspect, dietary plasmalogens have been studied after krill oil (KO) supplementation in humans, as described in Sung, et al., “Enrichment of n-3 containing ether phospholipids in plasma after 30 days of krill oil compared with fish oil supplementation,” Lipids (2022) 57: 115-124, which is incorporated by reference herein, in its entirety. It has been recognized that ether-containing phospholipids are minor components in plasma and include either alkyl or alkenyl phosphatidylcholine and phosphatidylethanolamine (the latter alkenyl classes being referred to as plasmalogens). It is further noted that while plasmalogen levels are lower than diacyl lipids, even so the plasmalogens are still quite abundant. These have only been studied recently because techniques such as lipidomics have allowed detection and quantitation of these and other minor lipid classes (see, e.g., Meikle, et al., “Postprandial plasma phospholipids in men are influenced by the source of dietary fat,” J Nutr. (2015) 145(9): 2012-2018). Methods of making certain ether lipids and/or plasmalogen compositions are described in WO 2021/007623 A1 to Baker Heart and Diabetes Institute, having inventor Meikle, which is incorporated by reference herein in its entirety. There are numerous approaches to the synthesis of these ether lipid compounds using either solely chemical synthesis or a combination of chemical and enzymatic synthesis strategies. Synthesis of AKG compounds have been described in the following papers: Carlos D. Magnusson, et al., “Chemoenzymatic synthesis of a focused library of enantiopure structured 1-O-alkyl-2,3-diacyl-sn-glycerol type ether lipids,” Tetrahedron (2011) 67: 1821-1836; and, Arnar Halldorsson, et al., “Lipase catalysed kinetic resolution of 1-O-alkylglycerols by sequential transesterification,” Tetrahedron: Asymmetry (2004) 15: 2893-2899. Similarly, synthesis of the lysoalkenylphosphatidylethanolamine (LPE(P)) from commercially available 2,3-O-isopropylidene-sn-glycerol has been described in Guanghui Ni, et al., “Synthesis and evaluation of immunostimulant plasmalogen lysophosphatidylethanolamine and analogues for natural killer T cells,” Bioorg. Med. Chem. (2014) 22(11): 2966-73. Gomes, MAGB, et al. describe the synthesis of numerous alkyl ether lipids (Gomes MAGB, Bauduin A, Le Roux C, Fouinneteau R, Berthe W, Berchel M, Couthon H, Jaffrès P A. “Synthesis of ether lipids: natural compounds and analogues,” Beilstein J Org Chem. 2023 Sep. 8; 19:1299-1369). FIG. 2 of Gomes loc cit. discusses a synthetic route that would be adaptable to production of the alkyl LPC(O) compounds of interest, providing the R-groups for the sn-1 fatty acid of interest and stopping at the lyso PAM step. One skilled in the art will appreciate that different protecting groups and/or deprotecting schemes can also be used. FIG. 6 of Gomes and further references disclosed therein describe how to produce the starting compound 1-O-alkylglycerol (i.e., compound 2.1 of FIG. 2 of Gomes). Additionally, U.S. Pat. No. 10,900,063, which is incorporated by reference herein in its entirety, describes the use of lipases to produce LPC(O)s.

### LPC(O) Synthetic Route

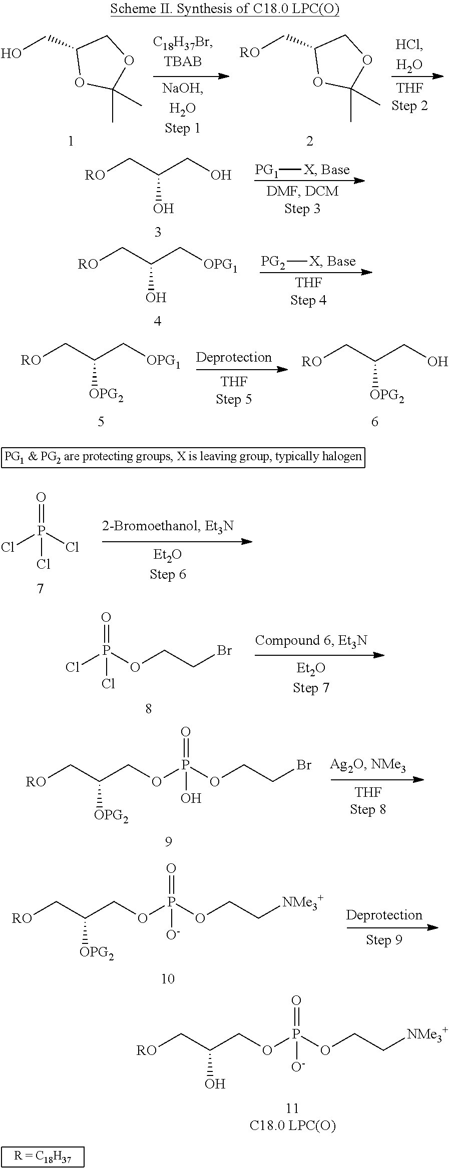

Starting material 1 (R-solketal or R-(−)-2,3-O-isopropylidene-sn-glycerol; CAS 14347-78-5; available from CombiBlocks (98% purity) or Fluorochem (95% purity)) is condensed with either alkyl bromides or mesylates to form corresponding alkyl substituted acetonides (Compound 2). TBAB shown in Scheme II above is tetrabutylammonium bromide, a commonly used phase transfer catalyst. The isopropylidene group can then be removed via acid hydrolysis (Compound 3), and alternative protecting groups applied to arrive at Compound 6. Separately, 2-bromoethyl phosphorodichloridate (Compound 8) is prepared, and then condensed with Compound 6 to give Compound 9. The terminal bromine atom is then replaced by trimethylamine, with a final alcohol deprotection step to afford Compound 11.

As would be readily understood by the skilled person, the reagents and conditions suggested above are merely exemplary and non-limiting. Synthesis of other SN-1 substituted LPC(O)s can be accomplished by, for example, providing appropriate alkyl bromide or alkyl mesylate compounds in step 1. For example, 1-bromooctadecane (CAS 112-89-0; available from TCI chemicals, >97.0% purity as determined by gas chromatography); oleyl mesylate (prepared by mesylation of oleyl alcohol: CAS 143-28-2; available from Sigma Aldrich, 85% purity as determined by gas chromatography); and 1-bromohexadecane (CAS 112-82-3; available from TCI chemicals, 96% purity as determined by gas chromatography) can be used in the synthesis of C18:0, C18:1 and C16:0 LCP(O)s respectively.

Suitable protecting groups for alcohol moieties are well known in the art (see, for example, Greene's Protective Groups in Organic Synthesis, Wiley, DOI: 10.1002/9781118905074).

### LPE(O) Synthetic Route

Synthesis of the corresponding LPE(O) compounds can be carried out in a similar manner using substituted ammonium compounds instead of trimethylamine in step 8. The ammonium compound can then be deprotected to leave an NH₃⁺ group. Such deprotection may take place alongside the alcohol deprotection in step 9, or via an additional synthetic step, depending on the nature of the substituents and protecting groups chosen. Alternative exemplary and non-limiting syntheses include:
(i) saturation of the vinyl double bond of LPE(P) alkenyl compounds produced by the method of Ni et al. loc cit, with chemical (typically nickel) or enzymatic reduction, which provides an alternative synthesis of LPE(O)s; and
(ii) hydrolysis of the SN2 group of commercially available alkyl PE compounds, such as 1-hexadecyl-2-(9Z-octadecenoyl)-sn-glycero-3-phosphoethanolamine (Cat 878130) from e.g. Avanti Polar Lipids (≤avantilipids.com>), using chemical or enzymatic means, such as an appropriate lipase to give C16.O alkyl LPE(O).

In a particular embodiment, a combination of lysoakylphosphatidylcholine (LPC(O)), or lysoalkylphosphatidylethanolamine (LPE(O)) compounds is contemplated. Derivatives of the aforementioned compounds are contemplated. One exemplary embodiment comprises a specific formulation of three species of lysoalkylphosphatidylcholine (LPC(O)). The species may include LPC(O-18:1), shown as compound (I-A1) below, LPC(O-18:0), compound (I-A2), and LPC(O-16:0), compound (I-A3). These ether lipids may have a molar ratio of R¹ groups of about 1.0:1.7:1.4, for example. The ratios may be varied among different cellular target populations and different subjects.

In this preferred embodiment, the compounds are depicted as follows, as LPC(O-18:1):

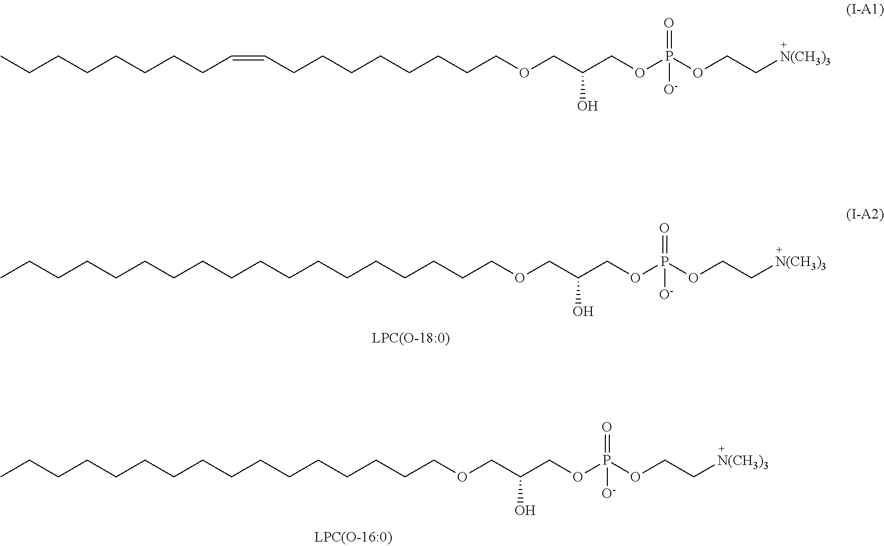

### (I-A3)

Other compounds and combinations are also contemplated, such as a combination of lysoalkylphosphatidylethanolamines (LPE(O)), including LPE(O-18:1), shown as compound (I-A4) below, LPE(O-18:0), compound (I-A5), and LPE(O-16:0), compound (I-A6). Molar ratios of R¹ groups may be varied, as well as molar percentages and weight percentages.

In this embodiment, the compounds are depicted as follows, as LPE(O-18:1):

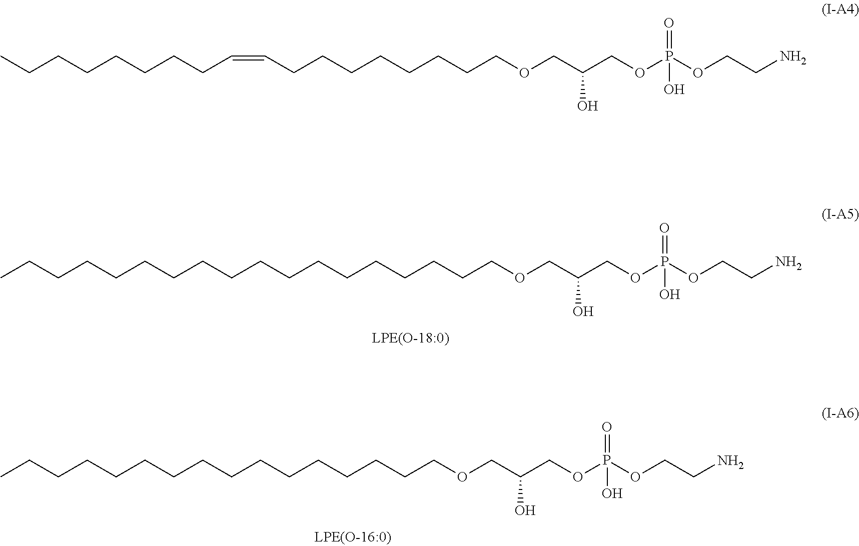 [SMILES: CCCCCCCC/C=C\CCCCCCCCOC[C@@H](O)COP(=O)(O)OCCN ; CCCCCCCCCCCCCCCCCCOC[C@@H](O)COP(=O)(O)OCCN ; CCCCCCCCCCCCCCCCOC[C@@H](O)COP(=O)(O)OCCN]

Other compounds and combinations are also contemplated, such as a combination of lysoalkylphosphatidic acids (LPA(O)), which are phosphate compounds, including LPA(O-18:1), shown as compound (I-A7) below, LPA(O-18:0), compound (I-A8), and LPA(O-16:0), compound (I-A9). Molar ratios of R¹ groups may be varied, as well as molar percentages and weight percentages.

In this embodiment, the compounds are depicted as follows, as LPA(O-18:1):

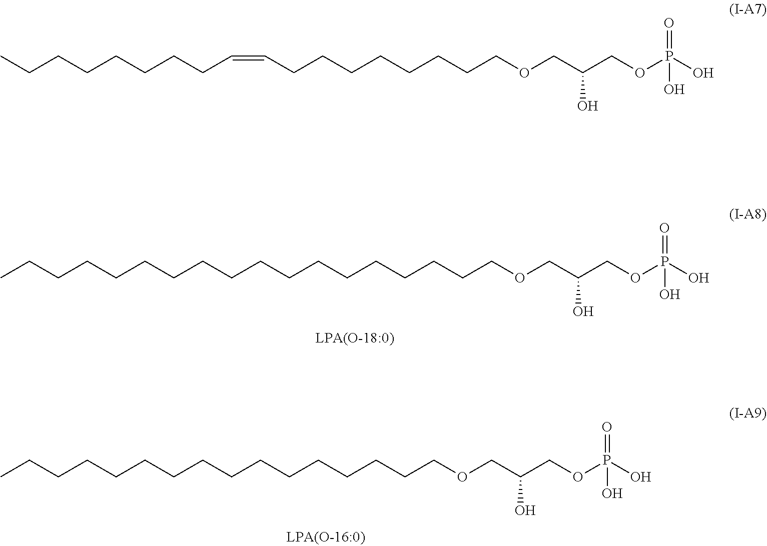 [SMILES: CCCCCCCC/C=C\CCCCCCCCOC[C@@H](O)COP(=O)(O)O ; CCCCCCCCCCCCCCCCCCOC[C@@H](O)COP(=O)(O)O ; CCCCCCCCCCCCCCCCOC[C@@H](O)COP(=O)(O)O]

When ingested, these phosphocholine species, in particular the LPC(O)s, are absorbed in the intestine and then metabolized into a series of ether phospholipids that include: alkyphosphatidylcholine (PC(O)), alkylphosphatidylethanolamine (PE(O)), alkenylphosphatidylcholine (plasmalogen, PC(P)) and alkenylphosphatidylethanolamine (plasmalogen, PE(P)). In studies, this led to increased levels of both the PC(P) and PE(P) species, which are bioactive species with a number of desirable health properties. The bioavailability and conversion of the LPC(O) into PC(P) and PE(P) is 5-12-fold higher than the commonly used alkylglycerol (AKG) species also used to increase plasmalogen levels. Without wishing to be bound by theory, it is believed that this means that lower doses can be used to achieve the same elevation of plasmalogens.

A further embodiment contemplated by the present disclosure is a formulation, which may be in the form of a food, such as a dietary supplement, that, upon ingestion leads to an increased level of plasmalogens within the blood and tissues of the recipient and thereby ameliorates or overcomes any plasmalogen deficiency that may exist within said individual. Increasing the levels of plasmalogens may lead to improved health outcomes. Such a formulation, for example, an LPC(O) formulation, could also be incorporated into a range of foods to facilitate delivery to the recipient. The intended recipients would be anyone who is deficient in plasmalogens and/or who is at risk of any of a range of metabolic diseases where plasmalogens may play a protective role.

As used herein, the term “dietary supplement” refers to a food product intended to enhance the diet of the subject and thereby improve nutrition. Dietary supplements may include the compositions described herein alone, or alongside other ingredients intended to supplement the diet, such as vitamins and minerals, fibre, herbs and other botanical extracts including flower remedies, homeopathic remedies, amino acids, enzymes and live microbials, probiotics, prebiotics or any combination thereof. Dietary supplements may be formulated in a wide variety of ways including as oils, gummies, drops, capsules, rapid-melt formulations, lozenges, oral sprays, chewing gums, gels, powders, premixed drinks, meal replacement shakes, or bars.

An example of a foodstuff into which a formulation as described herein can be incorporated, is infant formula. Foods in which a formulation comprising, for example, LPC(O)s can be incorporated include infant formula, follow-on formula, Medical Foods and Foods for Special Medical Purposes.

An example of a foodstuff into which a composition or formulation as described herein can be incorporated is any food stuff formulated for human consumption.

The terms “Medical Foods” or “Foods for Special Medical Purposes” as used herein refer to foodstuffs that are specially formulated and intended for the dietary management of a disease, disorder or condition that has distinctive nutritional needs that cannot be met by normal diet alone. Medical Foods assist patients who are temporarily or permanently unable to achieve an adequate nutritional intake from normal foods or through modification of the normal diet and are either malnourished or at risk of becoming malnourished. Medical Foods are used under medical supervision and may be administered orally or via tube feeds (e.g., nasogastric tubes). These terms as used herein are referred to under Regulation (EU) No 609/2013 and (Food and Drug Authority (FDA)) 21 CFR 101.9(j)(8)(ii). They are thus distinguished from dietary supplements which are generally available for consumption without medical supervision.

The term “infant” as used herein, is taken to mean a person aged 12 months or younger. The term “infant” is taken to additionally mean “pre-term infant”, which is a person aged 12 months or younger, who was born prior to 36 weeks of gestation. The term “toddler” as used herein, is taken to mean a person greater than one year of age up to three years of age. The term “child” or “children” as used herein, refers to a person greater than three years of age, up to 12 years of age. The terms “infant formula” as used herein, unless otherwise specified, refers to liquid, semi-liquid, solid and semi-solid human milk replacements or substitutes that are suitable for consumption by an infant. The synthetic formulas include components that are of semi-purified or purified origin. As used herein, the terms “semi-purified” and “purified” refer to a material that has been prepared by purification of a natural material or by synthesis. The term “infant formula” is not taken to include unmodified human breast milk.

The infant formula may include liquid and powdered dietary supplements, liquid and powdered human milk fortifiers, liquid and powdered preterm infant formulas, liquid and powdered infant formulas, liquid and powdered elemental and semi-elemental formulas, liquid and powdered toddler formulas, and powdered follow—on formulas suitable for use infants and children. Compositions may be in any product form comprising the ingredients described herein, and which is safe and effective for oral administration.

The infant formula may further include ingredients including, protein, fat, carbohydrate, vitamins, minerals, anti-caking agents, emulsifiers. In some embodiments, the formula may contain purified cow's milk whey and/or casein as a protein source, a blend of vegetable oils as a fat source, lactose as a carbohydrate source, a vitamin-mineral mix, and other ingredients including, but not limited to, any antioxidants suitable for oral administration such as vitamin A, vitamin E, vitamin C, retinal, tocopherol, L-ascorbyl palmitate, riboflavin and carotenoids, including lutein, beta-carotene, zeaxanthin, and lycopene, and combinations thereof. The infant formula may include oils, for example, vegetable oil including, for example, high oleic sunflower oil, coconut oil, canola oil, sunflower oil, or fish oil, and combinations thereof. The infant formula may include milk products and derivatives from milk, including, for example, lactose, milk proteins, galacto-oligosaccharides, whey concentrate, fructo-oligosaccharides, further compounds enriched from milk(s) secreted by mammals including, but not limited to human, bovine, etc.

The infant formula may include anti-caking agents such as tricalcium phosphate, potassium chloride, sodium citrate, and potassium citrate, magnesium hydrogen phosphate, and coagulants, such as for example magnesium chloride, choline chloride, L-ascorbic acid, emulsifier iron (II) sulfate, zinc sulfate.

The powders may be reconstituted with water prior to use to a caloric density tailored to the nutritional needs of the ultimate user, although in most instances the powders are reconstituted with water to form compositions comprising at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the reconstituted powder may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter.

When the nutritional product is a powdered infant formula, the protein component is present in an amount of from about 5% to about 35%, including from about 8% to about 12%, and including from about 10% to about 12% by weight of the infant formula; the fat component is present in an amount of from about 10% to about 35%, including from about 25% to about 30%, and including from about 26% to about 28% by weight of the infant formula; and the carbohydrate component is present in an amount of from about 30% to about 85%, including from about 45% to about 60%, including from about 50% to about 55% by weight of the infant formula.

The infant formulas contemplated herein may be formulated to include at least one of fat, protein, and carbohydrate, and preferably also contains vitamins, minerals, and at least one compound of Formula (I), (I-A), (I-A1), (I-A2), (I-A3), (I-A4), (I-A5), and/or (I-A6) or combinations thereof.

There are many disease settings where plasmalogens have been demonstrated to play a protective role. These include, but are not limited to: metabolic disorders (obesity, insulin resistance, type 2 diabetes; nonalcoholic fatty liver disease, nonalcoholic steatohepatitis); immune related diseases (asthma, atopic dermatitis, type 1 diabetes, infection); cardiovascular disease (atherosclerosis, cardiac remodeling, hypertension); neurological diseases (Alzheimer's disease; Parkinson's disease, autism, amyotrophic lateral sclerosis, frontotemporal dementia; multiple sclerosis; schizophrenia); cancer; myalgic encephalomyelitis/chronic fatigue syndrome; Barth syndrome; peroxisomal disorders (Zellweger syndrome spectrum disorders, rhizomelic chondrodysplasia punctata). These have been reviewed in several recent papers, as follows: Tremblay, et al., “Plasmalogens and platelet-activating factor roles in chronic inflammatory diseases,” BioFactors (2022) 1-14; Schooneveldt, et al., “Ether lipids in obesity: from cells to population studies,” Frontiers in Physiology (March 2022) 13: 1-11; Boselli, Jr., et al., “Plasmalogen replacement therapy,” Membranes (2021) 11: 838; and, S. Paul, G. I. Lancaster, and P. Meikle, “Plasmalogens: a potential therapeutic target for neurodegenerative and cardiometabolic disease,” Progress Lipid Res. (2019) 74: 186-195.

The formulation of the supplement is based on the proportional levels of the corresponding PE(P) species in circulation or tissues, and accordingly, specific formulations may be used to target specific fluids (e.g., plasma), tissues, such as liver, heart, or fat tissues, or specific cell types, such as immune cells. With regard to targeting plasma, an embodiment of a contemplated formulation comprises ether lipids having a molar ratio of 18:1 alkenyl/alkyl R¹ groups to 18:0 alkyl R¹ groups to 16:0 alkyl R¹ groups of about 1.0:1.7:1.4, having a molar percent of 18:1 ether groups in the range of from 18.6% to 27.9%, having a molar percent of 18:0 ether groups in the range of from 32.6% to 45.8%, or having a molar percent of 16:0 ether groups in the range of from 26.8% to 37.4%.

With regard to targeting immune cells, an embodiment of a contemplated formulation comprises ether lipids having a molar ratio of 18:1 alkenyl/alkyl R¹ groups to 18:0 alkyl R¹ groups to 16:0 alkyl R¹ groups is about 1.0:3.4:3.3.

An embodiment of a contemplated formulation comprises at least one compound of Formula (I). In certain embodiments, the at least one compound of Formula (I) is a compound of Formula (I-A). In certain embodiments, the at least one compound of Formula (I-A) is one or more of Formula (I-A1), Formula (I-A2) and/or Formula (I-A3). In certain embodiments, the at least one compound of Formula (I) is a mixture of two compounds of Formula (I). In certain embodiments, the at least one compound of Formula (I) is a mixture of two compounds of Formula (I-A). In certain embodiments, the at least one compound of Formula (I-A) is a mixture of two compounds selected from Formula (I-A1), Formula (I-A2) and Formula (I-A3). In certain embodiments, the at least one compound of Formula (I-A) is a mixture of Formula (I-A1), Formula (I-A2) and Formula (I-A3).

In certain embodiments, the mixture of Formula (I-A1), Formula (I-A2) and Formula (I-A3) makes up at least 50% of the ether lipids in the on a molar percentage basis. In certain embodiments, the mixture has a molar ratio of from 1.2:1 to 2.5:1 of (I-A2) to (I-A1). In certain embodiments, the mixture has a molar ratio of from 1.5:1 to 2.1:1 of (I-A2) to (I-A1). In certain embodiments, the mixture has a molar ratio of 1.7:1 of (I-A2) to (I-A1). In certain embodiments, the mixture has a molar ratio of from 0.9:1 to 1.7:1 of (I-A2) to (I-A3). In certain embodiments, the mixture has a molar ratio of from 1:1 to 1.5:1 of (I-A2) to (I-A3). In certain embodiments, the mixture has a molar ratio of 1.22:1 of (I-A2) to (I-A3). In certain embodiments, the mixture has a molar ratio of from 0.5:1 to 1:1 of (I-A1) to (I-A3). In certain embodiments, the mixture has a molar ratio of from 0.6:1 to 0.9:1 of (I-A1) to (I-A3). In certain embodiments, the mixture has a molar ratio of 0.72:1 of (I-A1) to (I-A3). In certain embodiments, the mixture has a molar percent of (I-A1) of from 18.6% to 27.9%, of (I-A2) of from 32.6% to 45.8%, and of (I-A3) of from 26.8% to 37.4%. In certain embodiments, the mixture has a molar percent of (I-A1) of 23.3%, a molar percent of (I-A2) of 39.2%, and a molar percent of (I-A3) of 32.1%. In certain embodiments, the mixture has a molar ratio of (I-A1):(I-A2):(I-A3) of 1:1.7:1.4.

In certain embodiments, the at least one compound of Formula (I), (I-A), (I-A1), (I-A2) and/or (I-A3) is converted to at least one plasmalogen in vivo.

An embodiment of a contemplated formulation comprises at least one compound of Formula (I). In certain embodiments, the at least one compound of Formula (I) is a compound of Formula (I-A). In certain embodiments, the at least one compound of Formula (I-A) is one or more of Formula (I-A4), Formula (I-A5) and/or Formula (I-A6). In certain embodiments, the at least one compound of Formula (I) is a mixture of two compounds of Formula (I). In certain embodiments, the at least one compound of Formula (I) is a mixture of two compounds of Formula (I-A). In certain embodiments, the at least one compound of Formula (I-A) is a mixture of two compounds selected from Formula (I-A4), Formula (I-A5) and Formula (I-A6). In certain embodiments, the at least one compound of Formula (I-A) is a mixture of Formula (I-A4), Formula (I-A5) and Formula (I-A6).

In certain embodiments, the mixture of Formula (I-A4), Formula (I-A5) and Formula (I-A6) makes up at least 50% of the ether lipids in the composition on a molar percentage basis. In certain embodiments, the mixture has a molar ratio of from 1.2:1 to 2.5:1 of (I-A5) to (I-A4). In certain embodiments, the mixture has a molar ratio of from 1.5:1 to 2.1:1 of (I-A5) to (I-A4). In certain embodiments, the mixture has a molar ratio of 1.7:1 of (I-A5) to (I-A4). In certain embodiments, the mixture has a molar ratio of from 0.9:1 to 1.7:1 of (I-A5) to (I-A6). In certain embodiments, the mixture has a molar ratio of from 1:1 to 1.5:1 of (I-A5) to (I-A6). In certain embodiments, the mixture has a molar ratio of 1.29:1 of (I-A5) to (I-A6). In certain embodiments, the mixture has a molar ratio of from 0.5:1 to 1:1 of (I-A4) to (I-A6). In certain embodiments, the mixture has a molar ratio of from 0.6:1 to 0.9:1 of (I-A4) to (I-A6). In certain embodiments, the mixture has a molar ratio of 0.76:1 of (I-A4) to (I-A6). In certain embodiments, the mixture has a molar percent of (I-A4) of from 18.6% to 27.9%, of (I-A5) of from 32.6% to 45.8%, and of (I-A6) of from 2.8% to 37.4%. In certain embodiments, the mixture has a molar percent of (I-A4) of 24.8%, a molar percent of (I-A5) of 42.4%, and a molar percent of (I-A6) of 32.8%. In certain embodiments, the mixture has a molar ratio of (I-A4):(I-A5):(I-A6) of 1:1.7:1.4.

In certain embodiments, the at least one compound of Formula (I), (I-A), (I-A4), (I-A5) and/or (I-A6) is converted to at least one plasmalogen in vivo.

In certain embodiments, the mixture of the at least one compound of Formula (I) or (I-A), is a mixture of compounds of (I-A1), (I-A2), (I-A3), (I-A4), (I-A5) and/or (I-A6).

It is expected that the above-disclosed ratios of the above formulations will produce optimized ratios of plasmalogens PE(P) and PC(P) in the serum and tissues of a human subject who is administered the formulations.

Other useful phosphatidyl classes can be prepared in accordance with the principles herein, for examples phosphatidyl serines, and phosphatidyl inositols.

In further embodiments, the LPC(O)s, LPE(O)s, and LPA(O)s may be used in various advantageous combinations which may be effective to increase plasmalogen levels. The plasmalogens which are potentiated or modulated are generally represented by those listed above.

In certain embodiments, the above-described formulations include one or more liquid or gel-based carriers, including, but not limited to, those selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents; thickening agents, including, but not limited to, those selected from the group consisting of corn starch, guar gum, xanthan gum, and the like. In certain embodiments, the one or more liquid or gel-based carrier(s) can be added to the formulations in a weight/volume percentage of from about 0.5% to about 95% weight/volume of the formulation. In certain embodiments, the natural or synthetic flavoring(s) can be added to the formulations in a weight/volume percentage of from about 3.0% to about 10.0% weight/volume of the formulation. In certain embodiments, the coloring agent(s) can be added to the formulations in a weight/volume percentage of from about 1.0% to about 10.0% weight/volume of the formulation. In certain embodiments, the thickening agent(s) can be added to the formulations in a weight/volume percentage of about 2% weight/volume of the formulation.

### Delivery System

The formulations disclosed herein may be delivered via dosage forms including, but not limited to, tablets, capsules, solutions, suspensions, powders, gums, and confectionaries. The formulations disclosed herein may be delivered via sublingual delivery systems including, but not limited to, dissolvable tabs under and on the tongue, liquid drops, and beverages. Alternatively, or in addition, edible films, hydrophilic polymers, oral dissolvable films, or oral dissolvable strips can be used.

For oral administration, the formulations disclosed herein may be further combined with one or more solid inactive ingredients for the preparation of tablets, capsules, pills, powders, granules, or other suitable dosage forms. For example, the formulation components may be combined with at least one excipient including, but not limited to, those selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like. In some embodiments, formulations according to the disclosure may include one or more of beeswax (such as beeswax E901), carnauba wax (such as carnauba wax E903), shellac (such as shellac E904), candelilla wax (such as candelilla wax E902), microcrystalline wax (such as microcrystalline wax E905), paraffin wax, and di-acylglycerols.

The components of the formulations administered according to the methods of the present disclosure can be administered in a wide variety of oral dosage forms. It will be obvious to those skilled in the art that suitable dosage forms may comprise, in certain embodiments one or more chemical compounds of the present disclosure and/or one or more pharmaceutically acceptable salts of a chemical compound of the present disclosure.

For preparing pharmaceutical formulations or compositions to be administered according to the methods of the present disclosure, pharmaceutically acceptable carriers can be either solid or liquid. Solid form preparations include powders, tablets, pills, capsules, and cachets. A solid carrier can be one or more substances that may also act as diluents, flavoring agents, solubilizing agents, lubricants, suspending agents, binders, preservatives, tablet disintegrating agents, or encapsulating materials.

In powders, the carrier is a finely divided solid, which is in a mixture with the finely divided active component. In tablets, one or more compounds of the present disclosure and/or pharmaceutically acceptable salts thereof is mixed with one or more carriers having the necessary binding capacity in suitable proportions, which is then compacted in the shape and size desired.

In certain embodiments, powders and tablets administered according to methods of the present disclosure preferably may contain, in total, from about one to about ninety-nine percent, such as from five or ten to about seventy percent one or more compounds of the present disclosure and/or pharmaceutically acceptable salts thereof. Suitable carriers include, but are not limited to, are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of one or more compounds of the present disclosure and/or pharmaceutically acceptable salts thereof with encapsulating material as a carrier providing a capsule in which one or more compounds of the present disclosure and/or pharmaceutically acceptable salts thereof, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, lozenges and rapid melts can be used as solid forms suitable for oral administration.

Capsules may be prepared in such a way as to be additionally coated for timed release. Coating thickness may be modified to provide a delayed release of the capsule contents. Capsules may be prepared in such a way as to be targeted release capsules. In some embodiments, the capsule can be targeted to the stomach. In some embodiments, the capsule targeted for delivery to the stomach is coated in a film coating. In some embodiments, the capsule can be targeted to the small intestine. In some embodiments, the capsule targeted for delivery to the small intestine is coated in an enteric coating.

The term “rapid-melt” as used herein refers to pharmaceutical formulations that melt on contact with saliva requiring little or no chewing.

Liquid preparations include, but are not limited to, solutions, suspensions, and emulsions, for example, water or water-propylene glycol solutions. The formulated preparations may take such forms as suspensions, solutions, or emulsions in oily or aqueous vehicles, and may contain formulation agents such as suspending, stabilizing, solubilizing, and/or dispersing agents. Alternatively, one or more compounds of the present disclosure and/or pharmaceutically acceptable salts thereof may be in powder form, such as that obtained by aseptic isolation of sterile solid or by lyophilization from solution, for constitution with a suitable vehicle, e.g., sterile, pyrogen-free water, before use.

Aqueous solutions suitable for oral use can be prepared by dissolving one or more compounds of the present disclosure, and/or pharmaceutically acceptable salts thereof, in water, and adding suitable colorants, flavors, stabilizing and thickening agents, as desired. Aqueous suspensions suitable for oral use can be made by solubilizing, and/or dispersing the finely divided active component in water with viscous material, such as natural or synthetic gums, resins, methylcellulose, sodium carboxymethylcellulose, or other well-known suspending agents.

Formulations suitable for topical administration in the mouth, or buccal, or sublingual administration include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

In some embodiments, a formulation comprises a solubilizing agent. As used herein, the term “solubilizing agent” refers to any agent which promotes solubilization or dispersal of the composition when placed into a liquid. In some embodiments, a “solubilizing agent” may be a dispersal agent. The solubilizing agent may additionally improve the stability of the formulated composition. Suitable solubilizing agents include, but are not limited to, carboxymethylcellulose sodium, hypromellose, proline, xanthan gum, maltodextrin, alginates, waxes, lipids, oils, alcohols, sugars, microcrystalline cellulose, starches, calcium phosphates, mannitol, sorbitol, erythritol, food grade solvents, phospholipids, such as lecithin (including, but not limited to, egg yolk L-α-lecithin, such as egg yolk L-α-lecithin available from Sigma-Aldrich, Saint Louis, M.O., U.S.A.), DMSO, ethanol, ethyl acetate, isopropanol, and the like. In some embodiments, a solubilizing agent is used to improve the separation of the compounds which make up the formulation and to prevent their settling or clumping in formulations. In some embodiments, a formulation comprises a solubilizing agent and a suitable carrier. In some embodiments, such a formulation is a pharmaceutical-grade product. In some embodiments, such a formulation is a food-grade product.

The pharmaceutical formulations or preparations are preferably in unit dosage forms. In such form, the formulation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules, and lozenges for oral administration and liquids for oral use are preferred formulations.

Further details on techniques for formulation and administration may be found in the latest edition of REMINGTON'S PHARMACEUTICAL SCIENCES (Mack Publishing Co., Easton, PA).

### Routes of Administration

The compounds of the present disclosure and/or pharmaceutically acceptable salts thereof may be administered by any route, including, but not limited to, oral, sublingual, buccal, or as an oral spray.

The methods described above may be further understood in connection with the following Examples. In addition, the following non-limiting examples are provided to illustrate the disclosure. However, the person skilled in the art will appreciate that it may be necessary to vary the procedures for any given embodiment of the disclosure, e.g., vary the order or steps.

### NUMBERED EMBODIMENTS

1. A composition comprising at least one compound of Formula (I) or a pharmaceutically acceptable salt thereof

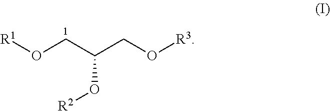

2. The composition of embodiment 1, wherein R³ is selected from phosphate and substituted phosphate.
3. The composition of embodiment 2, wherein R³ is substituted phosphate.
4. The composition of embodiment 3, wherein the substituted phosphate is substituted by an alkyl amine or inositol.
5. The composition of embodiment 4, wherein the alkyl amine is selected from

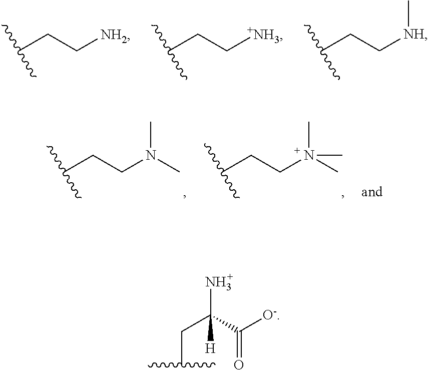

6. The composition of any one of embodiments 1-5, wherein R¹ and R² are each independently selected from hydrogen, an optionally substituted C₁₋₃₀ alkyl group, an optionally substituted C₂₋₃₀ alkenyl group, and an optionally substituted C₁₋₃₀ acyl group.
7. The composition of embodiment 6, wherein R¹ and R² are each independently selected from an optionally substituted C₁₄₋₂₄ alkyl group, an optionally substituted C₁₄₋₂₄ alkenyl group, and an optionally substituted C₁₄₋₂₄ acyl group.
8. The composition of embodiment 6, wherein if R² is hydrogen, R¹ is an optionally substituted C₁₋₃₀ alkyl group, an optionally substituted C₂₋₃₀ alkenyl group, or an optionally substituted C₁₋₃₀ acyl group.
9. The composition of embodiment 8, wherein R¹ is an optionally substituted C₁₄₋₂₄ alkyl group, an optionally substituted C₁₄₋₂₄ alkenyl group, or an optionally substituted C₁₄₋₂₄ acyl group.
10. The composition of embodiment 9, wherein R¹ is an optionally substituted C₁₄₋₁₈ alkyl group, an optionally substituted C₁₄₋₁₈ alkenyl group, or an optionally substituted C₁₄₋₁₈ acyl group.
11. The composition of embodiment 10, wherein R¹ is an unsubstituted C₁₆ alkyl group.
12. The composition of embodiment 10, wherein R¹ is an unsubstituted C₁₈ alkyl group.
13. The composition of embodiment 10, wherein R¹ is an unsubstituted C₁₈ alkenyl group.
14. The composition of embodiment 1, wherein the at least one compound of Formula (I) is a compound of Formula (I-A), or a pharmaceutically acceptable salt thereof

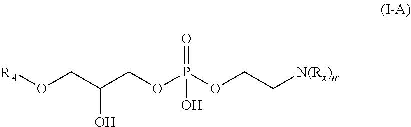

15. The composition of embodiment 14, wherein each Rₓ is independently selected from hydrogen or C₁₋₃ alkyl.
16. The composition of embodiment 14 or 15, wherein n is 2 or 3.
17. The composition of embodiment 14, wherein Rₓ is methyl, and n is 3.
18. The composition of any one of embodiments 14 to 17, wherein R, is an optionally substituted hydrocarbon chain containing from 14 to 24 carbon atoms.
19. The composition of embodiment 18, wherein R_{A} is an optionally substituted hydrocarbon chain containing from 16 to 18 carbon atoms.
20. The composition of embodiment 19, wherein R_{A} is an optionally substituted C₁₆₋₁₈ alkyl group, an optionally substituted C₁₆₋₁₈ alkenyl group, or an optionally substituted C₁₆₋₁₈ acyl group.
21. The composition of embodiment 20, wherein R_{A} is an optionally substituted C₁₆₋₁₈ alkyl group.
22. The composition of embodiment 20, wherein R_{A} is an optionally substituted C₁₆₋₁₈ alkenyl group.
23. The composition of embodiment 19, wherein R_{A} is an unsubstituted C₁₆ alkyl group.
24. The composition of embodiment 19, wherein R_{A} is an unsubstituted C₁₈ alkyl group.
25. The composition of embodiment 19, wherein R_{A} is an unsubstituted C₁₈ alkenyl group.
26. The composition of any preceding embodiment, wherein the composition comprises a mixture of at least two compounds of Formula (I) and/or (I-A).
27. The composition of any preceding embodiment, wherein the composition comprises a mixture of three compounds of Formula (I) and/or (I-A).
28. The composition of embodiment 14, wherein the compound of Formula (I-A) is of the structure (I-A1), (I-A2), or (I-A3)

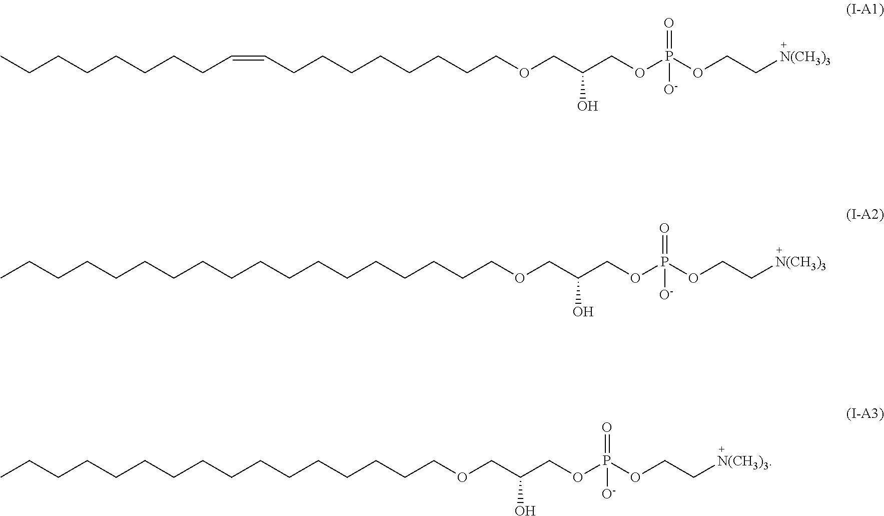

29. The composition of embodiment 28, wherein the composition comprises a mixture of at least two compounds selected from the group consisting of (I-A1), (I-A2), and (I-A3).
30. The composition of embodiment 28, wherein the composition comprises a mixture of (I-A1), (I-A2), and (I-A3).
31. The composition of embodiment 28, wherein the composition comprises a mixture of (I-A1), (I-A2), and (I-A3), wherein said mixture of (I-A1), (I-A2), and (I-A3) makes up at least 50% of the ether lipids in the composition on a molar percentage basis.
32. The composition of embodiment 28, wherein the mixture has a molar ratio of from 1.2:1 to 2.5:1 of (I-A2) to (I-A1).
33. The composition of embodiment 32, wherein the mixture has a molar ratio of from 1.5:1 to 2.1:1 of (I-A2) to (I-A1).
34. The composition of embodiment 33, wherein the mixture has a molar ratio of 1.7:1 of (I-A2) to (I-A1).
35. The composition of embodiment 28, wherein the mixture has a molar ratio of from 0.9:1 to 1.7:1 of (I-A2) to (I-A3).
36. The composition of embodiment 35, wherein the mixture has a molar ratio of from 1:1 to 1.5:1 of (I-A2) to (I-A3).
37. The composition of embodiment 36, wherein the mixture has a molar ratio of 1.22:1 of (I-A2) to (I-A3).
38. The composition of embodiment 28, wherein the mixture has a molar ratio of from 0.5:1 to 1:1 of (I-A1) to (I-A3).
39. The composition of embodiment 38, wherein the mixture has a molar ratio of from 0.6:1 to 0.9:1 of (I-A1) to (I-A3).
40. The composition of embodiment 39, wherein the mixture has a molar ratio of from 0.72:1 of (I-A1) to (I-A3).
41. The composition of embodiment 30, wherein the mixture has a molar percentage of (I-A1) of from 18.6% to 27.9% of (I-A2) of from 32.6% to 45.8%, and of (I-A3) of from 26.8% to 37.4%.
42. The composition of embodiment 30, wherein the mixture has a molar percentage of (I-A1) of 23.3%, a molar percentage of (I-A2) of 39.2%, and a molar percentage of (I-A3) of 32.1%.
43. The composition of embodiment 30, wherein the mixture has a molar ratio of (I-A1):(I-A2):(I-A3) of 1:1.7:1.4.
44. The composition of embodiment 14, wherein the compound of Formula (I-A) is of the structure (I-A4), (I-A5), or (I-A6)

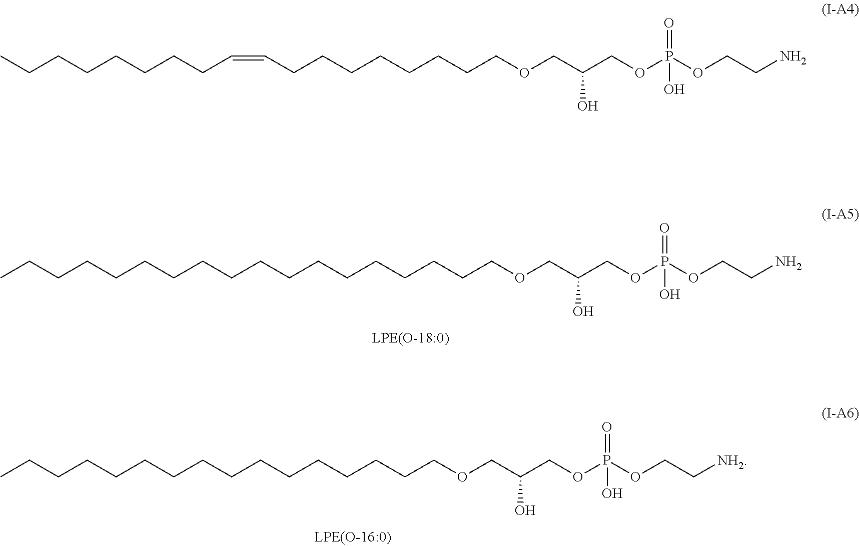 [SMILES: CCCCCCCC/C=C\CCCCCCCCOC[C@@H](O)COP(=O)(O)OCCN ; CCCCCCCCCCCCCCCCCCOC[C@@H](O)COP(=O)(O)OCCN ; CCCCCCCCCCCCCCCCOC[C@@H](O)COP(=O)(O)OCCN]

45. The composition of embodiment 44, wherein the composition comprises a mixture of at least two compounds selected from the group consisting of (I-A4), (I-A5), and (I-A6).
46. The composition of embodiment 44, wherein the composition comprises a mixture of (I-A4), (I-A5), and (I-A6).
47. The composition of embodiment 44, wherein the composition comprises a mixture of (I-A4), (I-A5), and (I-A6), wherein said mixture of (I-A4), (I-A5), and (I-A6) makes up at least 50% of the ether lipids in the composition on a molar percentage basis.
48. The composition of embodiment 44, wherein the mixture has a molar ratio of from 1.2:1 to 2.5:1 of (I-A5) to (I-A4).
49. The composition of embodiment 48, wherein the mixture has a molar ratio of from 1.5:1 to 2.1:1 of (I-A5) to (I-A4).
50. The composition of embodiment 49, wherein the mixture has a molar ratio of 1.7:1 of (I-A5) to (I-A4).
51. The composition of embodiment 44, wherein the mixture has a molar ratio of from 0.9:1 to 1.7:1 of (I-A5) to (I-A6).
52. The composition of embodiment 51, wherein the mixture has a molar ratio of from 1:1 to 1.5:1 of (I-A5) to (I-A6).
53. The composition of embodiment 52, wherein the mixture has a molar ratio of 1.29:1 of (I-A5) to (I-A6).
54. The composition of embodiment 44, wherein the mixture has a molar ratio of from 0.5:1 to 1:1 of (I-A4) to (I-A6).
55. The composition of embodiment 54, wherein the mixture has a molar ratio of from 0.6:1 to 0.9:1 of (I-A4) to (I-A6).
56. The composition of embodiment 55, wherein the mixture has a molar ratio of 0.76:1 of (I-A4) to (I-A6).
57. The composition of embodiment 46, wherein the mixture has a molar percentage of (I-A4) of from 18.6% to 27.9%, of (I-A5) of from 32.6% to 45.8%, and of (I-A6) of from 26.8% to 37.4%.
58. The composition of embodiment 57, wherein the mixture has a molar percentage of (I-A4) of 24.8%, a molar percentage of (I-A5) of 42.4%, and a molar percentage of (I-A6) of 32.8%.
59. The composition of embodiment 46, wherein the mixture has a molar ratio of (I-A4):(I-A5):(I-A6) of 1:1.7:1.3.
60. A formulation, such as a pharmaceutical formulation, comprising a composition as defined in any one of embodiments 1 to 59, and at least one excipient.
61. The formulation of embodiment 60, wherein the formulation is formulated for oral administration.
62. The formulation of embodiment 61, wherein the oral administration form is a tablet, a capsule, a solution, a mouthwash, a suspension, a powder, a gum, a confectionary, a lozenge, a sublingual delivery system, or a rapid-melt formulation.
63. The formulation of any one of embodiments 60 to 62, wherein the pharmaceutical formulation is a food product.
64. The formulation of embodiment 63, wherein the formulation is a dietary supplement.
65. The formulation of embodiment 63, wherein the food product is a medical food product.
66. The formulation of embodiment 63, wherein the food product is infant formula.
67. The formulation according to any one of embodiments 60 to 66 for maintaining or modulating the levels of plasmalogens in a human subject in need thereof.
68. The formulation according to any one of embodiments 60 to 66 for maintaining or modulating the levels of plasmalogens in an animal subject in need thereof.
69. The formulation according to embodiment 68, wherein said animal is selected from domestic animals, working animals and farm animals.
70. The formulation according to any one of embodiments 67 to 69, wherein said maintaining or modulating requires maintaining or modulating the levels of plasmalogens at levels and/or ratios associated with a non-disease state.
71. A composition as defined in any one of embodiments 1 to 59, or a formulation as defined in any one of embodiments 60 to 70 for use in increasing levels of plasmalogen compounds in the blood or tissues of a subject.
72. The composition for use according to embodiment 71, wherein said subject is a human.
73. The composition for use according to embodiment 71, wherein said subject is an animal.
74. The composition for use according to embodiment 73, wherein said animal subject is selected from domestic animals, working animals and farm animals.
75. A composition as defined in any one of embodiments 1 to 59, or a formulation as defined in any one of embodiments 60 to 70 for use in therapy.
76. A composition as defined in any one of embodiments 1 to 59, or a formulation as defined in any one of embodiments 60 to 70 for use in treating a disease or disorder associated with a deficiency in plasmalogens.
77. The composition or formulation for use according to embodiment 76, wherein said disease or disorder is a neurological disease.
78. The composition or formulation according to embodiment 77, wherein said neurological disease is selected from Alzheimer's disease, Parkinson's disease, autism, amyotrophic lateral sclerosis, frontotemporal dementia, multiple sclerosis, and schizophrenia.
79. The composition or formulation for use according to embodiment 76, wherein said disease or disorder is a metabolic disorder.
80. The composition or formulation according to embodiment 79, wherein the metabolic disorder is selected from the group consisting of obesity, insulin resistance, type 2 diabetes, nonalcoholic fatty liver disease, nonalcoholic steatohepatitis, immune related disorders, cardiovascular disease, neurological diseases, cancer, myalgic encephalomyelitis/chronic fatigue syndrome, Barth syndrome, and a peroxisomal disorder.
81. The composition or formulation according to embodiment 80, wherein the immune related disease is selected from the group consisting of asthma, atopic dermatitis, type 1 diabetes, and infection.
82. The composition or formulation according to embodiment 80, wherein the cardiovascular disease is selected from the group consisting of atherosclerosis, cardiac remodeling, and hypertension.
83. The composition or formulation according to embodiment 80, wherein the peroxisomal disorder is a Zellweger syndrome spectrum disorder or rhizomelic chondrodysplasia punctata.
84. The composition or formulation for use according to any one of embodiments 71 to 83, wherein the compound of Formula (I) or (I-A) is administered in a dose of from 0.1 to 4000 mg per day.
85. The composition or formulation for use according to embodiment 84, wherein the compound of Formula (I) or (I-A) is administered in a dose of from 0.1 to 2000 mg per day.
86. The composition or formulation for use according to embodiment 85, wherein the compound of Formula (I) or (I-A) is administered in a dose of from 25 to 1600 mg per day.
87. The composition or formulation for use according to embodiment 86, wherein the compound of Formula (I) or (I-A) is administered in a dose of 400 mg, 800 mg, or 1600 mg per day.
88. The composition or formulation for use according to embodiment 86, wherein the compound of Formula (I) or (I-A) is administered in a dose of 200 mg per day.
89. The composition or formulation for use according to embodiment 86, wherein the compound of Formula (I) or (I-A) is administered in a dose of from 25 to 100 mg per day.
90. A method of increasing levels of plasmalogen compounds in the blood or tissues of a subject in need thereof, comprising administering to said subject an effective amount of at least one compound of Formula (I) or a pharmaceutically acceptable salt thereof and/or at least one compound of Formula (I-A) or a pharmaceutically acceptable salt thereof as defined in embodiments 1 and 14 respectively.
91. The method of embodiment 90, wherein said subject is a human.
92. The method of embodiment 90, wherein said subject is an animal.
93. The method of embodiment 92, wherein said animal subject is selected from domestic animals, working animals and farm animals.
94. The method of embodiment 90, wherein the bioavailability of plasmalogen compounds in the blood or tissues of said subject is improved following administration of at least one compound of Formula (I) or a pharmaceutically acceptable salt thereof and/or at least one compound of Formula (I-A) or a pharmaceutically acceptable salt thereof relative to administration of AKG (alkylglycerol).
95. A method of treating a disease or disorder associated with a deficiency in plasmalogens, comprising administering to a subject in need thereof an effective amount of at least one compound of Formula (I) or a pharmaceutically acceptable salt thereof and/or at least one compound of Formula (I-A) or a pharmaceutically acceptable salt thereof as defined in embodiments 1 and 14 respectively.
96. The method of embodiment 95, wherein said subject is a human.
97. The method of embodiment 95, wherein said subject is an animal.
98. The method of embodiment 97, wherein said animal subject is selected from domestic animals, working animals and farm animals.
99. The method of embodiment 95, wherein said disease or disorder is a neurological disease.
100. The method of embodiment 99, wherein said neurological disease is selected from the group consisting of Alzheimer's disease, Parkinson's disease, autism, amyotrophic lateral sclerosis, frontotemporal dementia, multiple sclerosis, and schizophrenia.
101. The method of embodiment 95, wherein said disease or disorder is a metabolic disorder.
102. The method of embodiment 101, wherein the metabolic disorder is selected from the group consisting of obesity, insulin resistance, type 2 diabetes, nonalcoholic fatty liver disease, nonalcoholic steatohepatitis, immune related disorders, cardiovascular disease, neurological diseases, cancer, myalgic encephalomyelitis/chronic fatigue syndrome, Barth syndrome, and a peroxisomal disorder.
103. The method of embodiment 102, wherein the immune related disease is selected from the group consisting of asthma, atopic dermatitis, type 1 diabetes, and infection.
104. The method of embodiment 102, wherein the cardiovascular disease is selected from the group consisting of atherosclerosis, cardiac remodeling, and hypertension.
105. The method of embodiment 102, wherein the peroxisomal disorder is a Zellweger syndrome spectrum disorder or rhizomelic chondrodysplasia punctata.
106. The method of any one of embodiments 90 to 105, wherein the compound of Formula (I) or (I-A) is administered in a dose of from 0.1 to 4000 mg per day.
107. The method of embodiment 106, wherein the compound of Formula (I) or (I-A) is administered in a dose of from 0.1 to 2000 mg per day.
108. The method of embodiment 107, wherein the compound of Formula (I) or (I-A) is administered in a dose of from 25 to 1600 mg per day.
109. The method of embodiment 108, wherein the compound of Formula (I) or (I-A) is administered in a dose of 400 mg, 800 mg, or 1600 mg per day.
110. The method of any embodiment 108, wherein the compound of Formula (I) or (I-A) is administered in a dose of 200 mg per day.
111. The method of embodiment 108, wherein the compound of Formula (I) or (I-A) is administered in a dose of from 25 to 100 mg per day.
112. The method of embodiment 111, wherein the compound of Formula (I) or (I-A) is administered in a dose of 25 mg, 50 mg, or 100 mg per day.
113. Use of a composition as defined in any one of embodiments 1 to 59, or a formulation as defined in any one of embodiments 60 to 70 in the manufacture of a medicament for increasing levels of plasmalogen compounds in the blood or tissues of a subject in need thereof.
114. Use of a composition as defined in any one of embodiments 1 to 59, or a formulation as defined in any one of embodiments 60 to 70 in the manufacture of a medicament for the treatment of a disease or disorder associated with a deficiency in plasmalogens.
115. Use according to embodiment 114, wherein said disease or disorder is a neurological disease.
116. Use according to embodiment 115, wherein said neurological disease is selected from the group consisting of Alzheimer's disease, Parkinson's disease, autism, amyotrophic lateral sclerosis, frontotemporal dementia, multiple sclerosis, and schizophrenia.
117. Use according to embodiment 116, wherein said disease or disorder is a metabolic disorder.
118. Use according to embodiment 117, wherein the metabolic disorder is selected from obesity, insulin resistance, type 2 diabetes, nonalcoholic fatty liver disease, nonalcoholic steatohepatitis, immune related disorders, cardiovascular disease, neurological diseases, cancer, myalgic encephalomyelitis/chronic fatigue syndrome, Barth syndrome, and a peroxisomal disorder.
119. Use according to embodiment 118, wherein the immune related disease is selected from the group consisting of asthma, atopic dermatitis, type 1 diabetes, and infection.
120. Use according to embodiment 118, wherein the cardiovascular disease is selected from the group consisting of atherosclerosis, cardiac remodeling, and hypertension.
121. Use according to embodiment 118, wherein the peroxisomal disorder is a Zellweger syndrome spectrum disorder or rhizomelic chondrodysplasia punctata.
122. A kit for use in the method of any one of embodiments 90 to 112, said kit comprising at least one compound of Formula (I) or Formula (I-A) as defined in any one of embodiments 1 and 14 respectively or a pharmaceutically acceptable salt thereof.
123. The composition of embodiment 28, wherein the at least one compound is selected from a compound of Formula (I-A1), (I-A2) or (I-A3).
124. The composition of embodiment 29, wherein the mixture of at least two compounds is a 50:50 mixture.
125. The composition of embodiment 30, wherein the mixture of (I-A1), (I-A2) and (I-A3) is selected from:

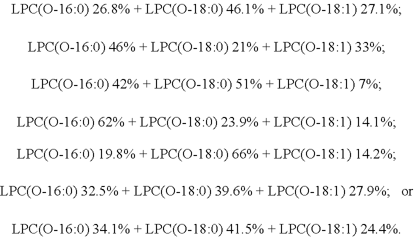

126. The composition of embodiment 44, wherein the at least one compound is selected from a compound of Formula (I-A4), (I-A5) or (I-A6).
127. The composition of embodiment 45, wherein the mixture of the at least two compounds is a 50:50 mixture.
128. The composition of embodiment 46, wherein the mixture of (I-A4), (I-A5) and (I-A6) is selected from:

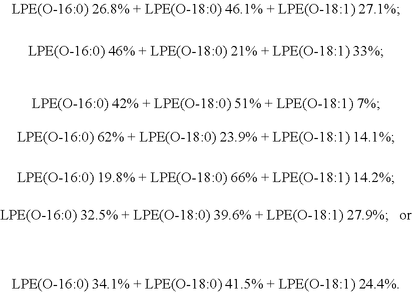

129. The composition of any one of embodiments 1-59 or 123-128, wherein the composition maintains or modulates the plasmalogen compound ratios observed in healthy human subjects.
130. The composition of any one of embodiments 1-59 or 123-128, wherein the composition results in a reduction in inflammation or the amelioration or reduction of symptoms associated with an inflammatory disease.
131. The composition of embodiment 130, wherein the reduction in inflammation is associated with a reduction in the levels of inflammatory cytokines.
132. The composition of any one of embodiments 1-59 or 123-128, wherein the composition maintains or modulates the levels of inflammatory cytokines at levels observed in healthy human subjects.
133. The composition of embodiment 132, wherein the inflammatory cytokine which is maintained or modulated is selected from IL-6, NFE2L2, TLR4.
134. A formulation comprising a composition as defined in any one of embodiments 1 to 59 or 123-128 and at least one excipient and/or solubilizing agent.
135. The formulation of embodiment 134, wherein the formulation is formulated for oral administration.
136. The formulation of embodiment 135, wherein the oral administration form is a beverage or a food product.
137. The formulation of embodiment 135, wherein the oral administration form is a food product.
138. The formulation of embodiment 135, wherein the oral administration form is infant formula.
139. The formulation of embodiment 134, wherein the formulation comprises a solubilizing agent.
140. The formulation of embodiment 139, wherein the solubilizing agent is selected from the group consisting of carboxymethylcellulose sodium, hypromellose, proline, xanthan gum, maltodextrin, alginates, waxes, lipids, oils, alcohols, sugars, microcrystalline cellulose, starches, calcium phosphates, mannitol, sorbitol, erythritol, food grade solvents, phospholipids, DMSO, ethanol, ethyl acetate, and isopropanol.
141. The formulation of embodiment 139, wherein the solubilizing agent is lecithin.
142. The formulation of embodiment 141, wherein the lecithin is purified egg yolk L-α-lecithin.
143. The formulation of embodiment 139, wherein the formulation has improved stability compared to a formulation comprising a composition as defined in any one of embodiments 1 to 59 or 123-128 that does not comprise a solubilizing agent.
144. The formulation of embodiment 139, wherein the formulation is less prone to degradation during freezing and thawing compared to a formulation comprising a composition as defined in any one of embodiments 1 to 59 or 123-128 that does not comprise a solubilizing agent.
145. The composition as defined in either one of embodiment 28 or embodiment 44, wherein the composition comprises one compound with a purity of at least 99.9%.
146. A formulation comprising a composition as defined in embodiment 145, and at least one excipient.
147. The formulation as defined any one of embodiments 134 to 138 or embodiment 146, wherein the formulation comprises at least one of a solubilizing agent, an emulsifier, a stabilizer, a dispersal agent, an antifoaming agent, or a diluent.
148. The formulation as defined any one of embodiments 134 to 138 or embodiments 146 to 147, wherein the formulation additionally comprises at least one solubilizing agent.
149. The formulation of embodiment 148, wherein the solubilizing agent is selected from the group consisting of carboxymethylcellulose sodium, hypromellose, proline, xanthan gum, maltodextrin, alginates, waxes, lipids, oils, alcohols, sugars, microcrystalline cellulose, starches, calcium phosphates, mannitol, sorbitol, erythritol, food grade solvents, phospholipids, DMSO, ethanol, ethyl acetate, and isopropanol.
150. The formulation of embodiment 149, wherein the solubilizing agent is selected from the group consisting of DMSO, ethanol, ethyl acetate, and isopropanol.
151. The formulation as defined any one of embodiments 134 to 144 or embodiments 146 to 150, wherein the formulation additionally comprises at least one antioxidant compound.
152. The formulation as defined any one of embodiments 134 to 144 or embodiments 146 to 151, wherein the formulation additionally comprises at least one anti-foaming agent.
153. The formulation as defined any one of embodiments 134 to 144 or embodiments 146 to 152, wherein the formulation is formulated for oral administration.
154. The formulation of embodiment 153, wherein the oral administration form is a tablet, a capsule, a solution, a mouthwash, a suspension, a powder, a gum, a confectionary, a lozenge, a sublingual delivery system, or a rapid-melt formulation.
155. The formulation of embodiment 154, wherein the oral administration form is a tablet.
156. The composition of embodiment 30, wherein the mixture of (I-A1), (I-A2) and (I-A3) comprises 90% w/v (I-A1).
157. The composition of embodiment 30, wherein the mixture of (I-A1), (I-A2) and (I-A3) comprises 90% w/v (I-A2).
158. The composition of embodiment 30, wherein the mixture of (I-A1), (I-A2) and (I-A3) comprises 90% w/v (I-A3).
159. The composition of embodiment 46, wherein the mixture of (I-A4), (I-A5) and (I-A6) comprises 90% w/v (I-A4).
160. The composition of embodiment 46, wherein the mixture of (I-A4), (I-A5) and (I-A6) comprises 90% w/v (I-A5).
161. The composition of embodiment 46, wherein the mixture of (I-A4), (I-A5) and (I-A6) comprises 90% w/v (I-A6).

The following nonlimiting examples are shown below. Two studies were compared to determine the effects of precursor compounds and various formulations of said precursor compounds on plasmalogen levels in humans.

### Example 1

The first study showed the effect krill and fish oil supplementation has on plasmalogen levels in female subjects.

The details of the lipidomic analyses have been described fully in K. Huynh, et al., “High-Throughput Plasma Lipidomics: Detailed Mapping of the Associations with Cardiometabolic Risk Factors,” Cell Chem. Biol. (2019) 26(1):71-84, and H H. Sung, et al., “Differential plasma postprandial lipidomic responses to krill oil and fish oil supplementations in women: A randomized crossover study,” Nutrition (2019) 65:191-201. Briefly, plasma samples were extracted in CHCl₃:MeOH (2:1) together with an internal standard mix containing non-physiological or stable isotope-labelled lipid standards, as previously described. Lipidomic analysis was performed by UHPLC ESI-MS/MS, using an Agilent 1290 HPLC coupled to an Agilent 6490 triple quadrupole mass spectrometer. Chromatographic data were analyzed using Mass Hunter Quant where relative lipid abundances were calculated by relating the area under the chromatogram for each lipid species to the corresponding internal standard. Correction factors were applied to adjust for different response factors, where these were known. Species that were chromatographically separated were labelled as such (e.g., PC(16:0/22:6) and PC(18:2/20:4)), whereas species that were mixed isomers were given the standard phospholipid notation (e.g., PC(40:8) was a mixture of PC(20:4/20:4) and PC(18:2/22:6). Where structural details were sufficient, lipids were manually annotated as containing long-chain omega-3 components (i.e., 20:5 EPA, 22:5 DPA, and 22:6 DHA).

This study was a randomized crossover study with krill oil (KO) and fish oil (FO) supplementation for 30 days and separated by a minimum washout period of 4 weeks between supplementations. Participants were instructed to maintain their habitual diet and were requested to not to consume any food or supplements containing omega-3 PUFA more than once a week during the study periods. For interventions, participants consumed daily either 7 one-gram capsules of KO (Euphausia superba oil, Swisse Wellness Pty Ltd., Victoria, Australia) containing 1.27 g of LC omega-3 PUFA (0.76 g EPA, 0.42 g DHA, 0.09 g DPA) or 5 one-gram capsules of FO (Natural FO, Swisse Wellness Pty Ltd., Victoria, Australia) containing 1.44 g of LC omega-3 PUFA (0.79 g EPA, 0.47 g DHA, 0.18 g DPA) for 30 days each. Participants were required to attend the clinic three times for blood and data collection, at days 0 (baseline), 15, and 30 for each supplementation period for blood sample collection. Prior to each clinic visit, participants consumed a low-fat evening meal, and were advised to avoid drinking alcohol and strenuous physical activities, and to fast from 10 pm. On each study day, standardized procedures were performed where participants arrived at the clinic between 7 am to 9 am, and a fasting blood sample (10 mL) was collected via a venipuncture by a qualified phlebotomist. After the blood sample collection, all participants completed a 24-h dietary recall and an electronic PUFA FFQ. The study protocol was approved by the Ethics Committee of Victoria University Human Research (HRE15-031). Informed consent was obtained from all participants prior to the study (ACTRN 12615000472572).

### Example 2

The second study showed the effect shark liver oil (SLO) supplementation has on plasmalogen levels in male subjects.

This study was a double-blind, placebo-controlled crossover study, where participants (n=10) were overweight or obese (BMI in the range of 28-40 kg/m²) adult males (aged 25-60 years) with no signs of cardiovascular disease or diabetes. Written informed consent was obtained from all study participants prior to commencement of the study. This study was performed in accordance with the ethical principles set forth in the Declaration of Helsinki and received approval from the Alfred Hospital Ethics Committee (approval number: 436/15). Participants were randomized into placebo or treatment arms and received 4 g Alkyrol® (purified SLO; Eurohealth, Ireland) per day or placebo (methylcellulose) for 3 weeks followed by a 3-week washout phase and were then crossed over to 3 weeks of the alternate placebo/Alkyrol® treatment. Fasting blood samples were collected at the start and end of each intervention. Tables 3 and 4 show the composition of alkyldiacylglycerols [TG(O)] and alkylglycerols (AKG), respectively, in Alkyrol® shark liver oil.

Table 3. The composition of alkyldiacylglycerols [TG(O)] in Alkyrol® shark liver oil.

TABLE 3

| TG(O) | Concentration (mM) | % of total TG(O) |
|---|---|---|
| TG(O-50:1) | 20.9 | 4.2 |
| TG(O-52:1) | 20.3 | 3.5 |
| TG(O-52:2) | 191.3 | 36.4 |
| TG(O-53:2) | 12.3 | 2.1 |
| TG(O-54:1) | 4.8 | 0.9 |
| TG(O-54:2) | 61.8 | 10.7 |
| TG(O-54:3) | 77.6 | 14.5 |
| TG(O-56:2) | 31.7 | 5.7 |
| TG(O-56:3) | 55.7 | 9.5 |
| TG(O-58:2) | 15.8 | 2.8 |
| TG(O-58:3) | 40.4 | 7.1 |
| TG(O-60:3) | 14.7 | 2.6 |

Table 4. The composition of alkylglycerols (AKG) in Alkyrol® shark liver oil.

TABLE 4

| AKG | Concentration (mM) | % of total AKG |
|---|---|---|
| AG(14:0) | 6.2 | 1.1 |
| AG(14:1) | 1.4 | 0.2 |
| AG(15:0) | 3.2 | 0.6 |
| AG(15:1) | 5.1 | 0.9 |
| AG(16:0) | 83.2 | 14.6 |
| AG(16:1) | 51.1 | 9.0 |
| AG(17:0) | 10.3 | 1.8 |
| AG(17:1) | 14.4 | 2.5 |
| AG(18:0) | 33.1 | 5.8 |
| AG(18:1) | 341.6 | 59.9 |
| AG(19:0) | 2.4 | 0.4 |
| AG(19:1) | 5.3 | 0.9 |
| AG(20:0) | 1.3 | 0.2 |
| AG(20:1) | 12.0 | 2.1 |

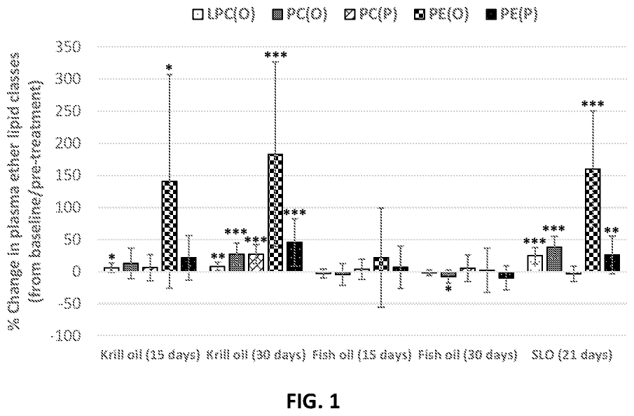

The datasets of the studies of Examples 1 and 2 were compared and the effect of these oil supplementations on plasma ether lipid levels were extrapolated. FIG. 1 and FIG. 2 show that both KO and SLO supplementation substantially increased the levels of different ether lipid classes in plasma while FO supplementation did not have much impact on these lipid classes. The increase in plasmalogens (PE(P) and PC(P)) were noticeably higher with KO supplementation (30 days) compared to SLO supplementation (21 days).

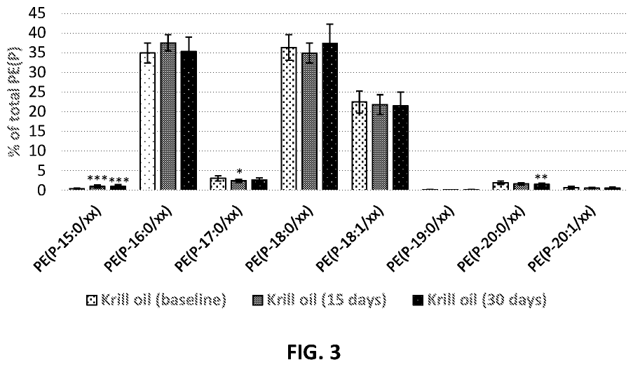

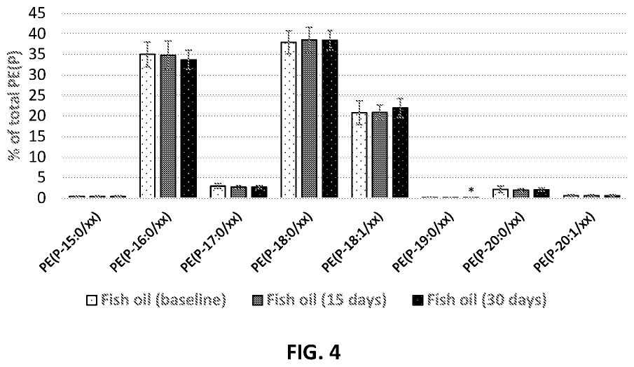

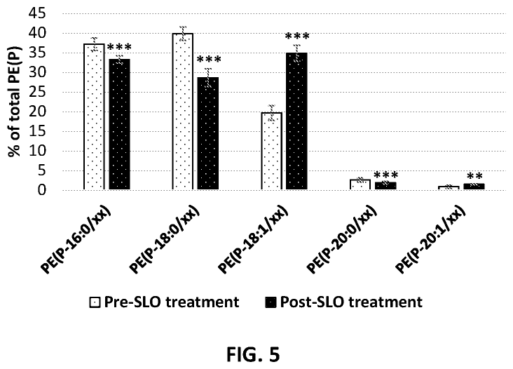

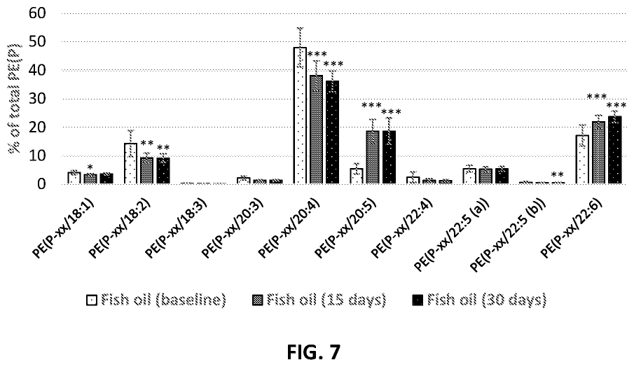

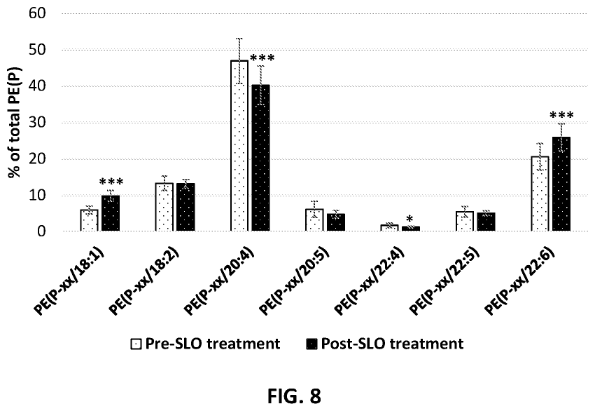

The effect of supplementation on PE(P) composition was also extrapolated. FIG. 3 and FIG. 4 show that both KO and FO supplementation did not have any noticeable impact on alkenyl chain composition of PE plasmalogen, while FIG. 5 shows that SLO supplementation significantly altered this composition (increased the proportion of 18:1 alkenyl chain containing PE plasmalogens and decreased the proportions of 16:0 and 18:0 alkenyl chain containing PE plasmalogens). All these supplementations had substantial effect on the acyl chain composition of PE plasmalogens. FIG. 6 and FIG. 7 show that both KO and FO supplementation decreased the proportion of 18:1, 18:2 and 20:4 containing PE plasmalogens and increased the proportions of 20:5 and 22:6 containing PE plasmalogens while FIG. 8 shows that SLO supplementation decreased 20:4 containing PE plasmalogens and increased the proportions of 18:1 and 22:6 containing PE plasmalogens.

Without wishing to be bound by theory, it is believed that these differences may arise from the types and content of ether lipids found in KO and SLO. In KO, the ether lipids are mostly phosphatidylcholine ether lipids (PC(O) or LPC(O)) whereas SLO ether lipids are monoalkyl-diacylglycerols or TG(O)). It was found that KO contains around 25 mg of LPC(O)/g of oil, while SLO contains around 200 mg of alkylglycerols/g of oil. Additionally, the alkyl chain composition of KO and SLO are distinct. In SLO, the most abundant alkyl species is O-18:1 (Paul, et al., Journal of Lipid Research (2021) 62: 10009) whereas in KO the most abundant alkyl species is O-16:0 (Table 4).

Table 5. The composition of lysoalkylphosphatidylcholines [LPC(O)] in krill oil. Several major components are presented in bold and underlined.

TABLE 5

| Lipid species | % of total LPC(O) |
|---|---|
| LPC(O-16:0) | 38.44 |
| LPC(O-16:1) | 22.65 |
| LPC(O-14:0) | 17.63 |
| LPC(O-18:1) | 7.46 |
| LPC(O-15:0) | 5.11 |
| LPC(O-18:2) | 3.19 |
| LPC(O-18:0) | 2.89 |
| LPC(O-17:0) | 1.56 |
| LPC(O-20:1) | 0.65 |
| LPC(O-22:1) | 0.22 |
| LPC(O-20:0) | 0.12 |
| LPC(O-24:2) | 0.03 |
| LPC(O-22:0) | 0.02 |
| LPC(O-24:1) | 0.02 |

The relative bioavailability of KO and SLO for ether lipid modulation was further compared, in Table 6. Considering the changes in ether lipid levels, KO LPC(O)s seem to have significantly higher bioavailability than SLO alkylglycerols.

Table 6. Differences in bioavailability between krill oil and shark liver oil.

TABLE 6

| | Mean % change in plasma ether lipid level | | | |
|---|---|---|---|---|
| | PE(O) | PE(P) | PC(O) | PC(P) |
| Shark liver oil (21 | 160 | 26 | 38 | −3 |
| days) | | | | |
| Krill oil (30 | 183 | 46 | 27 | 27 |
| days) | | | | |
| | Mean % change in plasma ether lipid level/umol dose exposed | | | |
| | PE(O) | PE(P) | PC(O) | PC(P) |
| Shark liver oil (21 | 5.2 | 0.8 | 1.2 | NC |
| days)* | | | | |
| Krill oil (30 | 41.8 | 10.4 | 6.3 | 6.2 |
| days)** | | | | |
| | Fold difference in bioavailability | | | |
| | PE(O) | PE(P) | PC(O) | PC(P) |
| KO vs SLO | 8.1 | 12.4 | 5.1 | NC |
| | Mean % change in plasma ether lipid level/umol dose exposed/week | | | |
| | (adjusting for the differences in treatment duration between the two | | | |
| | studies | | | |
| | PE(O) | PE(P) | PC(O) | PC(P) |
| Shark liver oil (21 | 1.7 | 0.3 | 0.4 | NC |
| days)* | | | | |
| Krill oil (30 | 9.7 | 2.4 | 1.5 | 1.4 |
| days)** | | | | |
| | Fold difference in bioavailability | | | |
| | PE(O) | PE(P) | PC(O) | PC(P) |
| KO vs SLO | 5.7 | 8.7 | 3.6 | NC |

NC = not calculated as there was no increase in PC(P) level with shark liver oil supplementation.
*Dose of shark liver oil/day: 4 g shark liver oil-800 mg alkylglycerol; Approximate molecular weight of alkylglycerols is 344 g/mol which gives 2.33 mmol/75 kg = 31 μmol/kg body weight. The treatment duration was 21 days.
**Dose of krill oil/day: 7 g krill oil =175 mg LPC(O); Approximate molecular weight of LPC(O) is 480 g/mol which gives 0.36 mmol/75 kg = 4.38 μmol/kg body weight. The treatment duration was 30 days.

It was observed that KO provided a twelve-fold higher percentage change of PE(P) level per micromolar dose, compared to SLO and that this effect was largely retained (eight-fold higher) when adjusted for the treatment period. This is a large improvement in the bioavailability of the LPC(O)/PC(O) in krill oil relative to the alkyldiacylglycerol present in SLO.

### Example 3

Animal experimentation was carried out by Product Safety labs (Dayton, NJ, USA). A total of 32 animals (all females) were used. Animals were selected based on adequate body weight gain, absence of clinical signs of disease or injury. Selected rats were randomized according to stratification by body weight so that group mean body weights do not differ by more than 20% within sex across groups and assigned to the following test groups (Table 7). Animals had ad libitum access to food and water throughout the experiment. Each animal was dosed by oral intubation using a stainless-steel ball-tipped gavage needle attached to an appropriate syringe. Dose administration was done once on Day 1.

Table 7. Study design

TABLE 7

| | No. | | Dose amount | Dose volume |
|---|---|---|---|---|
| | Animals/ | | of test lipid | (For oral |
| Group | Group | Group Identification | (Absolute, mg) | gavage, mL) |
| 1 | 4 | AKG dose mix C | 14 | 3.0 |
| 2 | 4 | AKG dose mix D | 47 | 3.0 |
| 3 | 4 | AKDAG-oleic dose | 36 | 3.0 |
| | | mix C | | |
| 4 | 4 | AKDAG-oleic dose | 120 | 3.0 |
| | | mix D | | |
| 5 | 4 | AKDAG-DHA dose | 40 | 3.0 |
| | | mix C | | |
| 6 | 4 | AKDAG-DHA dose | 133 | 3.0 |
| | | mix D | | |
| 7 | 4 | LPC(O) dose mix C | 21 | 3.0 |
| 8 | 4 | LPC(O) dose mix D | 70 | 3.0 |

### Plasmalogen Precursor Compounds and Formulations

Stable isotope versions of four different lipid species were administered at 2 doses (C, D) (Table 8). All these compounds were synthesized by Anthem Biosciences (Bengaluru, India).

Table 8. Deuterium labelled plasmalogen precursor compounds used in the study. “D2” indicates that two hydrogen atoms in the compound have been replaced by deuterium atoms (examples are provided in Scheme III below).

TABLE 8

| | | Dose | Dose Mix |
|---|---|---|---|
| Compound group | Composition | Mix C | D |
| Alkylglycerol | D2-C18.1-AKG (deuterium labeled C18:1 | 14 mg | 47 mg |
| (AKG) | alkyl glycerol)-25% (w/w) | | |
| | D2-C18.0-AKG (deuterium labeled C18:0 | | |
| | alkyl glycerol)-42.7% (w/w) | | |
| | D2-C16.0-AKG (deuterium labeled C16:0 | | |
| | alkyl glycerol)-32.3% (w/w) | | |
| Alkyldiacylglycerol | D2-C18.1/[Oleic]2 AKDAG (deuterium | 36 mg | 120 mg |
| (AKDAG) with | labeled C18:1 alkyl diacylglycerol with oleic | | |
| oleic acid | acid at sn-2, sn-3)-24.6% (w/w) | | |
| | D2-C18.0/[Oleic]2 AKDAG (deuterium | | |
| | labeled C18:0 alkyl diacylglycerol with oleic | | |
| | acid at sn-2, sn-3)-42% (w/w) | | |
| | D2-C16.0/[Oleic]2 AKDAG (deuterium | | |
| | labeled C16:0 alkyl diacylglycerol with oleic | | |
| | acid at sn-2, sn-3)-33.4% (w/w) | | |
| AKDAG with DHA | D2-C18.1/[DHA]2 AKDAG (deuterium | 41 mg | 137 mg |
| | labeled C18:1 alkyl diacylglycerol with | | |
| | docosahexaenoic acid at sn-2, sn-3)-24.6% | | |
| | (w/w) | | |
| | D2-C18.0/[DHA]2 AKDAG (deuterium | | |
| | labeled C18:0 alkyl diacylglycerol with | | |
| | docosahexaenoic acid at sn-2, sn-3)-41.9% | | |
| | (w/w) | | |
| | D2-C16.0/[DHA]2 AKDAG (deuterium | | |
| | labeled C16:0 alkyl diacylglycerol with | | |
| | docosahexaenoic acid at sn-2, sn-3)-33.5% | | |
| | (w/w) | | |
| LPC(O) | D2-C18.1 LPC(O) (deuterium labeled C18:1 | 21 mg | 70 mg |
| | lyso-alkylphosphatidylcholine)-24.8% (w/w) | | |
| | D2-C18.0 LPC(O) (deuterium labeled C18:0 | | |
| | lyso-alkylphosphatidylcholine)-42.3% (w/w) | | |
| | D2-C16.0 LPC(O) (deuterium labeled C16:0 | | |
| | lyso-alkylphosphatidylcholine)-32.9% (w/w) | | |

To facilitate the administration of the precursor compounds, purified egg yolk L-α-lecithin (Sigma-Aldrich, Saint Louis, MO, USA) was used as a vehicle (14 mg for dose mix C and 47 mg for dose mix D). Stock solutions of individual precursors (100 mg/ml) were firstly prepared in chloroform:methanol (1:1). A stock solution of lecithin (100 mg/ml; Sigma-Aldrich, Saint Louis, MO, USA) was also prepared in chloroform:methanol (1:1). To prepare the precursor compound mix, appropriate volumes of precursor compound and lecithin solutions were mixed, dried under a stream of nitrogen gas at 40° C. and then reconstituted in deionised water by vigorous vertexing, then sonication in an Ultrasonic Cleaner water bath (Soniclean, Adelaide, SA, Australia) for 1 hr and further sonication using a Misonix S-4000 Sonicator (Thermo Fisher Scientific, Melbourne, VIC, Australia) for 2×30 s at amplitude 25.

### Blood Sample Collection

Blood samples were collected at 8 time points (pre-dose, 1 hr, 2 hr, 4 hr, 8 hr, 12 hr, 24 hr and 48 hr post-dose) from all the animals. Approximately 200 μL of blood were collected sublingually under isoflurane anesthesia into blood collection tubes containing K2EDTA and kept on ice until centrifuged. Following centrifugation, the plasma was transferred to clean tubes and frozen at approximately −80° C. until analyzed.

### Terminal Sacrifice and Tissue Collection

Faecal samples from the 24 and 48-hour period were collected at terminal sacrifice, all surviving animals were euthanized using CO₂ asphyxiation. At 48 hr, the brain, liver, spleen, kidney, heart, gastrocnemius, and abdominal adipose tissues from all study animals were carefully dissected, placed on a fresh weigh boat and weighed wet, and then snap frozen within 5 minutes and stored at −80° C. No other observations were made on any animals at necropsy.

Lipid Extraction from Plasma Samples

Lipids were extracted form 10 μl of plasma samples using butanol:methanol (1:1) as described previously (Alshehry Z H. Et al 2015). Lipid extractions were performed as a single batch, with quality control samples (pooled plasma QC, NIST QCs and blanks) included every 20 samples.

Tissue Processing and Extraction of Lipids from Tissue Samples

Approximately 40-60 mg of tissues were homogenised in 400-600 μL of ice-cold phosphate buffered saline using a TissueLyser II (Qiagen, USA) for 60 s and then sonicated with a Misonix S-4000 Sonicator (Thermo Fisher Scientific, Melbourne, VIC, Australia) for 10-15 s at amplitude 25. Protein content of the homogenates was quantified using a Pierce™ BCA protein assay kit (Thermo Fisher Scientific, Rockford, IL, USA). Homogenates were then made up to a stock protein concentration of 5 mg/mL protein and 10 μL aliquots from the stock solutions, containing 50 μg of protein, were subsequently used for lipid extraction. For adipose tissues, 10 μL aliquots from the stock homogenates were used for lipid extraction. Lipids were extracted following single-phase chloroform:methanol (2:1) extraction procedure as described previously (Miekle P J. et al. 2011). Lipid extractions were performed as a single batch for each tissue type—excluding liver and brain samples which were extracted together, with quality control samples (pooled plasma QC, pooled tissue QC, NIST QCs, and blanks) included approximately every 10 samples.

### Liquid Chromatography-Mass Spectrometry Analysis

Lipidomic analysis was carried out on an Agilent 1290 UHPLC system, with a column system set to 45° C. (ZORBAX eclipse plus C18 column: 2.1×100 mm, 1.8 mm, Agilent), and an Agilent 6495 triple quadrupole mass spectrometer. Samples were placed in autosampler tray (20° C.) and 1 μL was injected. Lipids were separated with a stepped linear gradient of solvent A (50% water/30% acetonitrile/20% isopropanol) and solvent B (1% water/9% acetonitrile/90% isopropanol), both containing 10 mM ammonium formate, at a flow rate of 0.4 mL/min. The solvent gradient started at 85% A, decreasing to 50% over 2.5 min, to 43% over 0.1 min, to 30% over 6.4 min, to 7% over 0.1 min, to 4% over 1.9 min, then to 0% over 0.1 min and held for 0.9 min, then increased back to 85% over 0.2 min and held for equilibration for 3.8 min. The following mass spectrometer conditions were used; gas temperature, 150° C., gas flow rate 17 L/min, nebulizer 20 psi, sheath gas temperature 200° C., capillary voltage 3500V and sheath gas flow 10 L/min.

A modified version of a previously reported MRM method (see K. Huynh, et al., “High-Throughput Plasma Lipidomics: Detailed Mapping of the Associations with Cardiometabolic Risk Factors,” Cell Chem. Biol. (2019) 26(1):71-84) was used to detect deuterium incorporation into lipid species of the ether phospholipid and ether glycerolipid classes: alkyldiacylglycerol [TG(O)], lysoalkylphosphatidylcholine [LPC(O)], alkylphosphatidylcholine [PC(O)], alkylphosphatidylethanolamine [PE(O)], alkenylphosphatidylethanolamine [PE(P)] or PE plasmalogen, alkenylphosphatidylcholine [PC(P)] or PC plasmalogen. Both the deuterated and endogenous non-labelled versions of lipids were measured. The lipids with the deuterated isotope are denoted by [+1] and [+2], whereas the endogenous lipids are referred to as [+0]. Precursors labelled with D2 produce both [+1] and [+2] lipid species due to the location of the deuterium, making them susceptible to removal by the Δ1 desaturase (PEDS1) enzyme when producing plasmalogens. The precursor mass of the lipid species labelled with D2 was offset by 2 Da, as one deuterium is equivalent to 1 Da. The product ions were offset based on lipid class fragmentation patterns.

The Agilent MassHunter Quantitative Analysis software was used to integrate the peaks and quantify the area under the curve of each lipid species. To calculate the relative concentration of the deuterium labelled lipids in the samples, we first subtracted the signal for lipid species in the blanks from the samples. This removes any background signal that the mass spectrometer has captured. Next, we calculated the isotope ratio in the 0 hr samples (for tissues we used control male samples), which corresponds to the area of deuterium labelled lipids over the area of the endogenous lipids. This calculates the natural isotope ratio of each lipid that should be removed. We then multiplied the isotope ratio by the background subtracted area for each lipid to get the endogenous signal for each lipid. This value was then subtracted from all the deuterium labelled lipid areas. Afterwards, we divided this value by the area of the internal standard of the corresponding lipid class and multiplied it by the amount of internal standard added to the sample. This provided the relative concentration of the deuterated lipid in the sample.

### Effect of Labelled Precursor Supplementation on the Plasma Ether Lipids

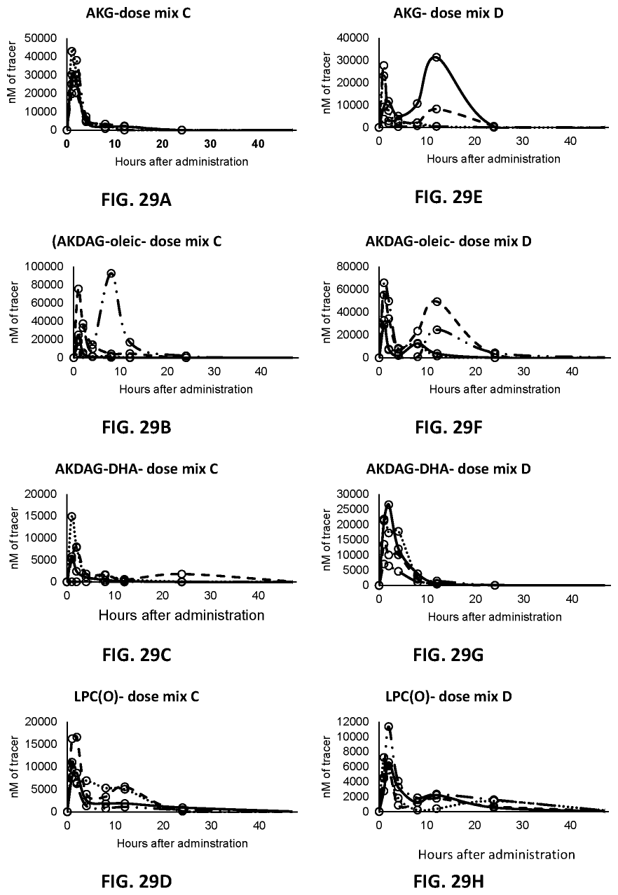

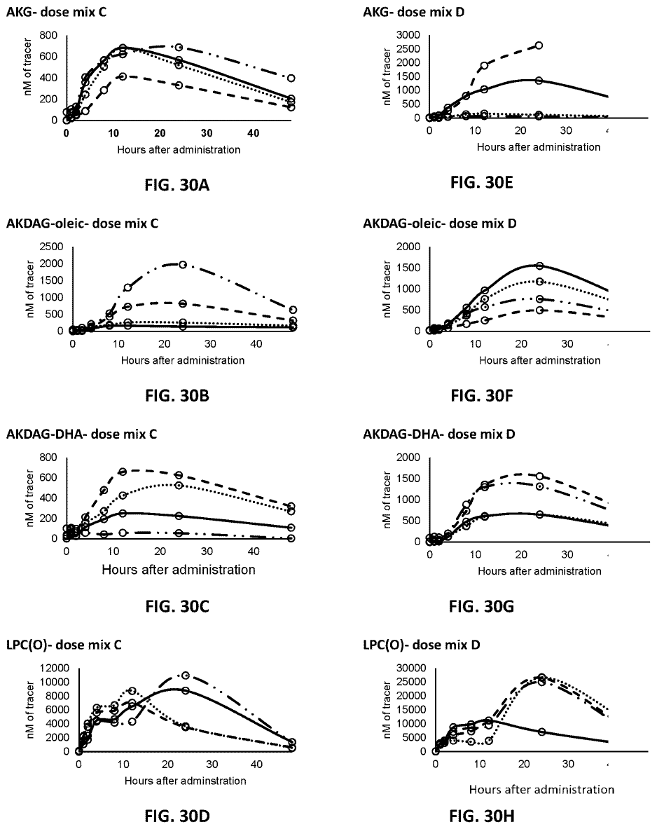

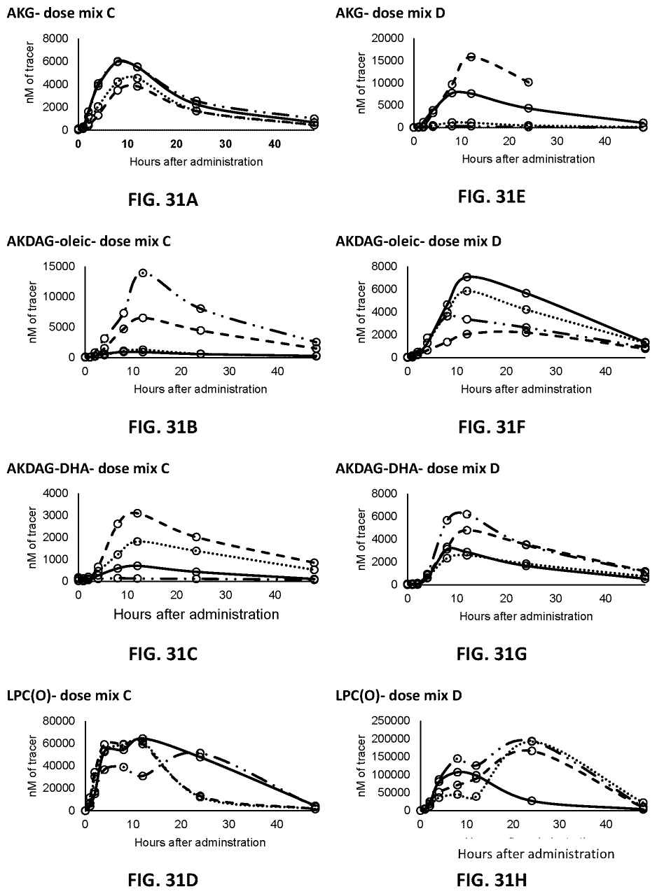

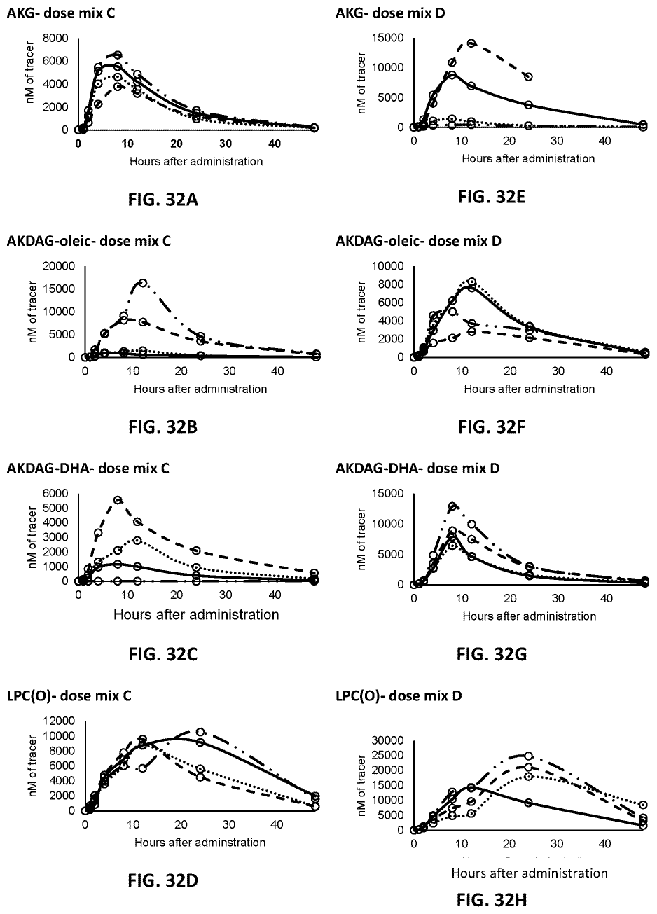

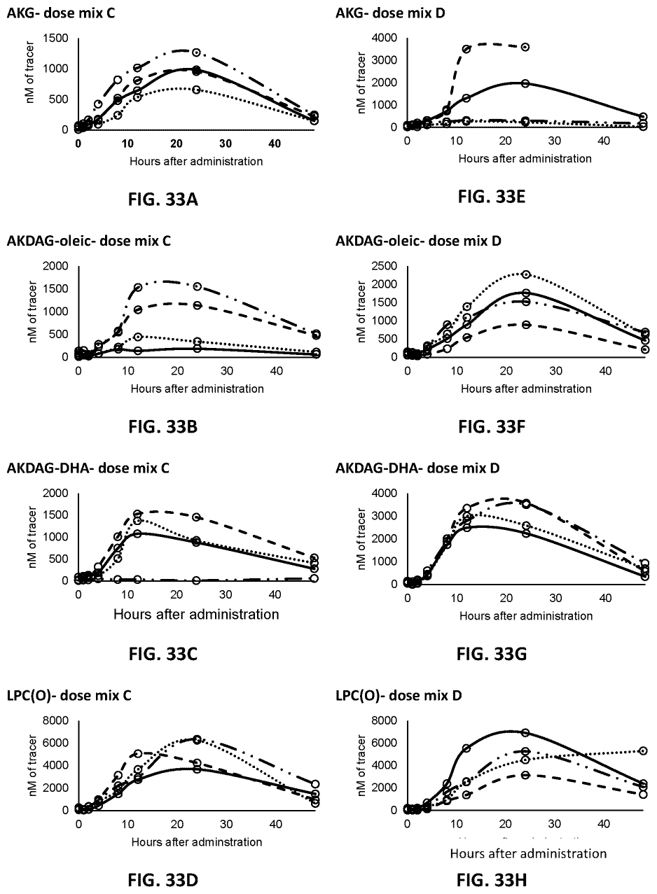

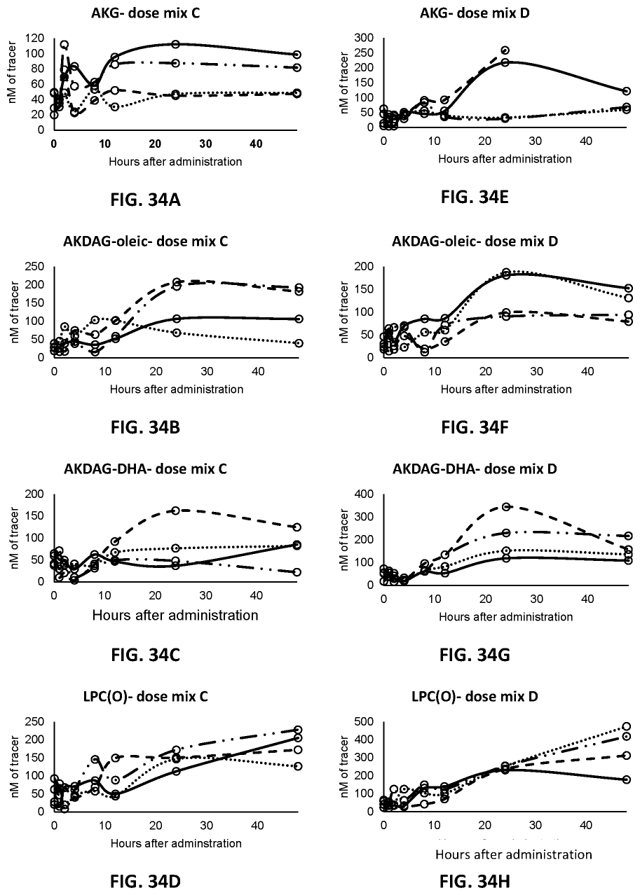

We observed distinct patterns in the concentrations of various tracer lipids over 48 hours following oral administration of the precursor compounds. Specifically, the concentration of TG(O) reached its highest point within 1-2 hours of administration, and then rapidly declined with all treatment groups (FIG. 29A-FIG. 29H). In the case of PC(O) (FIG. 31A-FIG. 31H), it took approximately 8-12 hours to reach the maximum concentration for all groups except LPC(O)-dose mix D (FIG. 30H) (24 hours). Interestingly it took much longer (12-24 hours) to reach the peak for LPC(O) concentration with all treatment groups, followed by a gradual decline (FIG. 30A-FIG. 30H). For PE(O) (FIG. 32A-FIG. 32H) the trajectory was much steeper (rapid rise withing 8 hours and then quick drop) for AKDAG-DHA treatment compared to others (FIG. 30C and FIG. 30G). The trajectory was much slower for PE(P), (FIG. 33A-FIG. 33H) notably, LPC(O) exhibited a much slower trajectory compared to other groups, with its concentration peaking at the 24-hour mark and then gradually subsiding (FIG. 33D and FIG. 33H), however showing a significant amount of tracer PE(P) at the 48-hour time point. The concentration of tracer PC(P) (FIG. 34A-FIG. 34H) peaked at 24 hours for all groups except LPC(O) (FIG. 34D and FIG. 34H) after fluctuations at earlier time points and remained stable up to 48 hours. For the LPC(O) groups, there was gradual increase in tracer PC(P) concentration starting from 12 to 48 hours. These observations highlight differing timelines for the peaks and subsequent declines in lipid concentrations across the treatments, shedding light on their differential dynamics of absorption and metabolism.

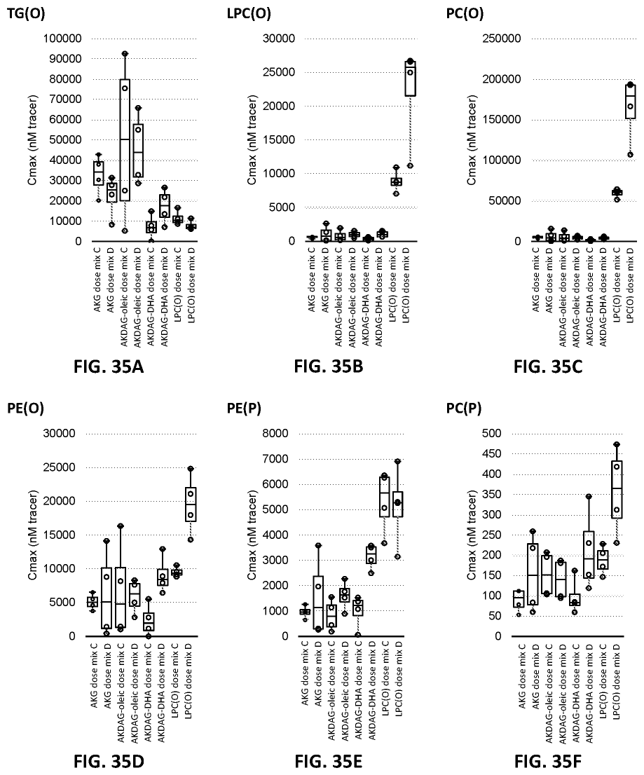

Next, we compared the maximum plasma tracer concentration, Cmax of major ether lipid classes among different experimental groups (FIG. 35A-FIG. 35F). We observed that the highest Cmax was consistently associated with LPC(O) groups across multiple lipid classes except for TG(O) (FIG. 35A). As expected, we observed a dose-dependent increase in plasma LPC(O) level following treatment with LPC(O) (FIG. 35B). Furthermore, similar dose-dependent patterns were observed for PC(O) (FIG. 35C) and PE(O) (FIG. 35D) within the LPC(O) groups. Additionally, the LPC(O) groups exhibited remarkably higher Cmax for PE(P) (FIG. 35E) compared to the other groups. The dose mix D LPC(O) group also displayed elevated Cmax for PC(P) (FIG. 35F).

Although LPC(O) was much more efficient in PE(P) production than other compounds, the actual conversion was low. LPC(O) dose mix C treatment shows 1.4% conversion into PC(O) and present in plasma at Cmax (FIG. 35C), but only 0.12% converted into PE(P) at Cmax and present in plasma (FIG. 35F).

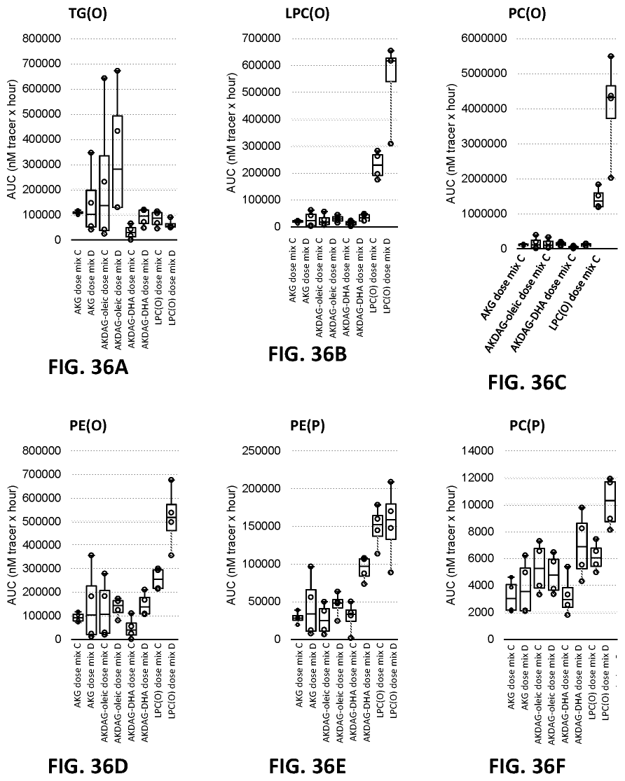

Next, we calculated the total exposure of the labelled precursor compounds as area under the curve (AUC) using the tracer lipid concentrations at different time points (FIG. 36A-FIG. 36F). We didn't observe significant difference in AUC among AKG, AKDAG with oleic acid [AKDAG(OA)], and AKDAG with DHA [AKDAG(DHA)] groups in PE(P) production (FIG. 36E), particularly evident in the case of PE(P) for dose mix Cs of each compound. However, LPC(O) administration (FIG. 36B) led to an approximately 5-fold increase in PE(P) relative to AKG and AKDAG based on the comparison between dose mix C groups. Intriguingly, LPC(O) displayed rapid metabolism into PC(O), resulting in a remarkable 16-fold elevation in PC(O) levels (FIG. 36C), accompanied by a comparatively modest 3-fold rise in PE(O) (FIG. 36D). This observation is interesting and indicates PC(O) as a metabolic sink for the LPC(O). Unlike LPC(O), AKG and AKDAG(OA) treatments exhibited rapid metabolism into TG(O) and showed much less conversion into PC(O), PE(O), and PE(P). On the other hand, AKDAG(DHA), especially at dose mix Ds, demonstrated a notable conversion into PE(P), albeit much lower than LPC(O). Notably, the production of PC(P) (FIG. 36F) remained limited across all treatments within a 2-day timeframe.

### Effect of Labelled Precursor Supplementation on the Tissue Ether Lipids

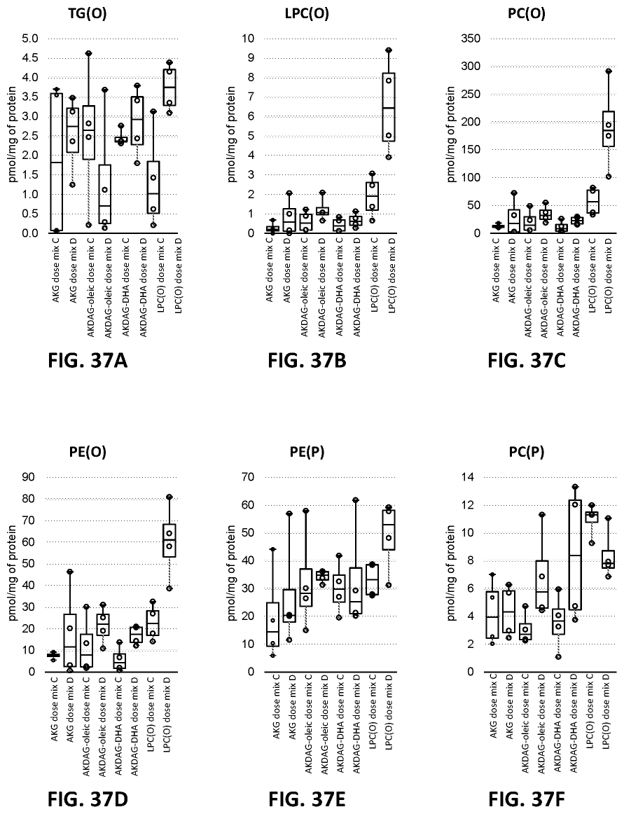

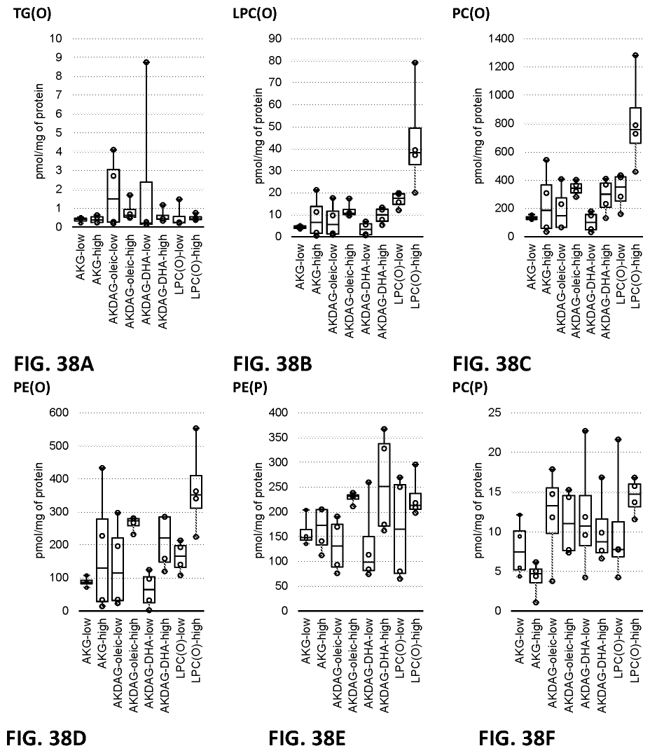

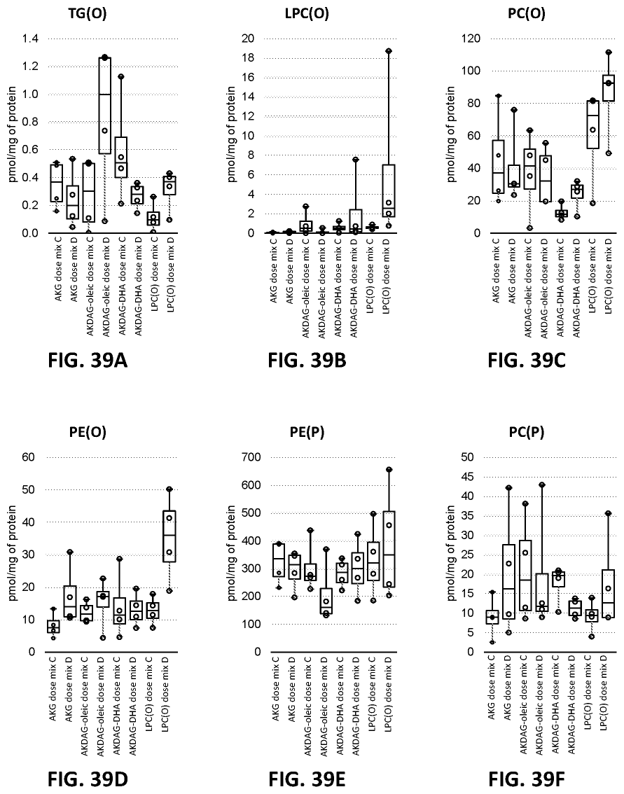

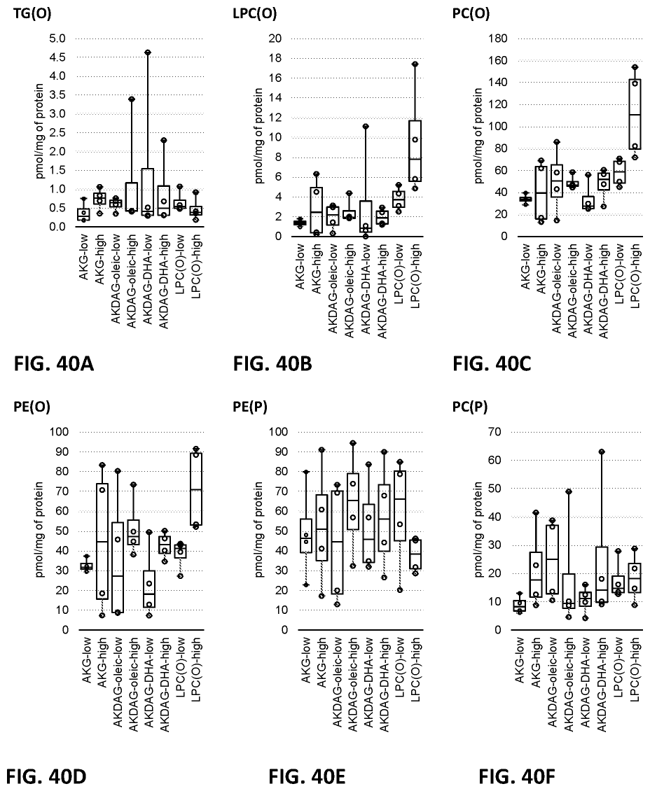

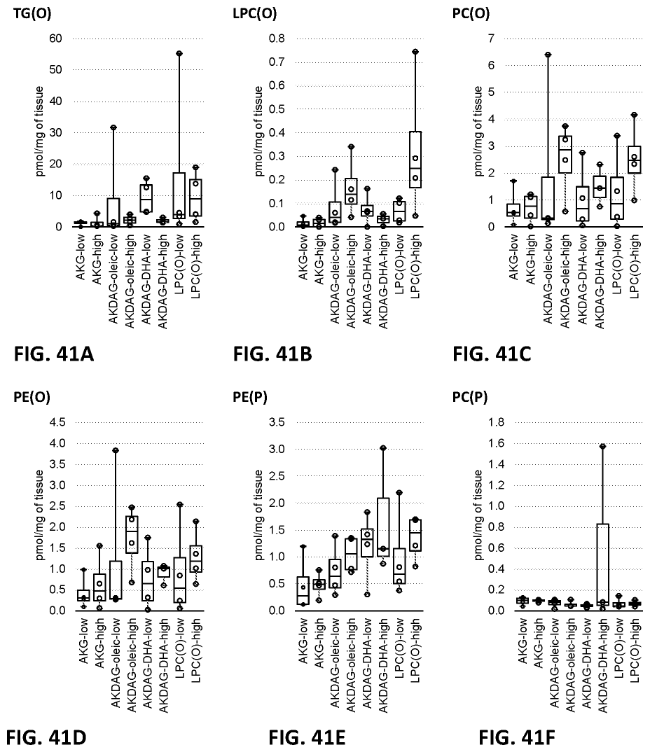

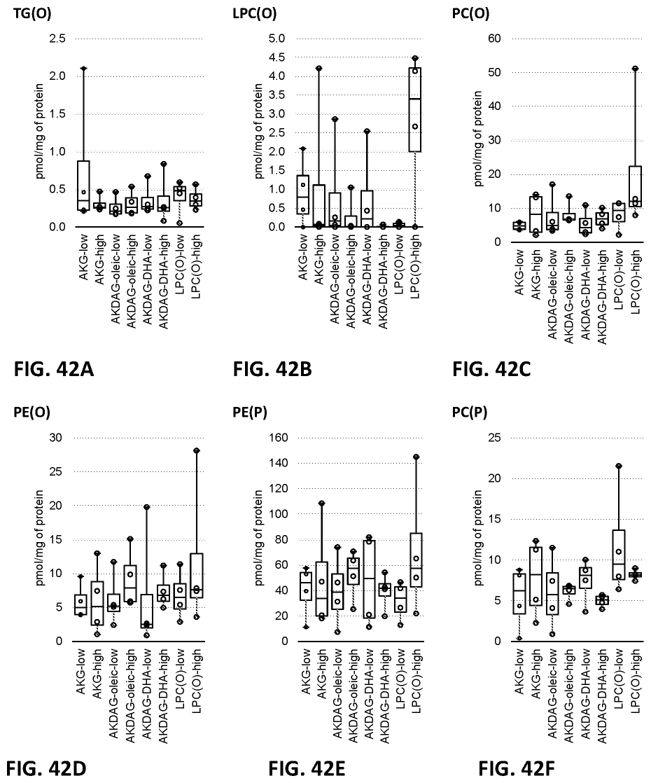

In addition to the analysis of plasma lipids, we conducted an examination of tissue ether lipids to assess the extent of incorporation of labelled precursors into endogenous ether lipids. Our observations indicate that in the liver, spleen, brain, and kidney, the labelled precursors were effectively converted into newly synthesized LPC(O), PC(O) and PE(O), particularly with the administration of LPC(O) (FIG. 37A-FIG. 43F). We did not observe obvious incorporation of labelled precursors into endogenous ether lipids within the visceral adipose tissue, skeletal muscle, and heart (FIG. 41A-FIG. 43F).

### Example 4

### Plasmalogen Precursor Treatment

Plasmalogen precursors were used to treat cells and mice to compare their bioavailability and conversion into endogenous plasmalogens. Precursors labelled with different numbers of deuterium atoms at various locations were used due to their availability. The number of deuterium-labelled sites is described using the letter “D” followed by a number. For example, “D2” refers to a precursor labelled with two deuterium atoms (see Scheme III). For example, cells were treated with AKG (O-16:0, D2) (Anthem Biosciences, Bengaluru, India), LPC(O-16:0, D4) (Cayman Chemical, Ann Arbor, USA) and LPE(O-16:0, D5) (Avanti Polar Lipids, Birmingham, USA) (Table 9). These are deuterium labelled plasmalogen precursors containing a 16:0 alkenyl chain (Scheme III). In comparison, mice were administered a mix of AKG (O-16:0, O-18:0, O-18:1, D2) (Anthem Biosciences, Bengaluru, India), a mix of LPC(O-16:0, O-18:0, O-18:1, D2) (Anthem Biosciences, Bengaluru, India), and LPE(O-16:0, D5) (Avanti Polar Lipids, Birmingham, USA) (Table 9). AKG and LPC(O) mixes were deuterium labelled and included 16:0, 18:0 and 18:1 alkenyl chains (Scheme III). LPE(O) contained a mixture of deuterium labelled 16:0 alkenyl chains and unlabeled 16:0, 18:0, 18:1 alkenyl chains (Scheme III).

Table 9. Composition of the plasmalogen precursor treatment for HepG2 cells, 3T3 cells and mice. Cells and mice were treated with control, alkylglycerol (AKG), lysoalkylphosphatidylcholine (LPC(O)), lysoalkylphosphatidylethanolamine (LPE(O)). Media for cells included Dulbecco Modified Eagle's Medium (DMEM).

TABLE 9

| Compound | Composition for | Composition for 3T3 | |
|---|---|---|---|
| group | HepG2 cells | cells | Composition for mice |
| Control | DMEM with 1% sodium | DMEM with 2% bovine | Lecithin (vehicle control) |
| | pyruvate and 0.05% | serum albumin and 0.05% | |
| | ethanol | ethanol | |
| AKG | Deuterium labelled D2- | Deuterium labelled D2- | Deuterium labelled D2-C18:1- |
| | C16:0-AKG in DMEM | C16:0-AKG in DMEM with | AKG-25% (w/w) |
| | with 1% sodium pyruvate | 2% bovine serum albumin | Deuterium labelled D2-C18:0- |
| | | | AKG-42.7% (w/w) |
| | | | Deuterium labelled D2-C16:0- |
| | | | AKG-32.3% (w/w) |
| LPC(O) | Deuterium labelled D4- | Deuterium labelled D4- | Deuterium labelled D2-C18:1 |
| | C16:0-LPC(O) in DMEM | C16:0-LPC(O) in DMEM | LPC(O)-24.8% (w/w) |
| | with 1% sodium pyruvate | with 2% bovine serum | Deuterium labelled D2-C18:0 |
| | | albumin | LPC(O)-42.3% (w/w) |
| | | | Deuterium labelled D2-C16:0 |
| | | | LPC(O)-32.9% (w/w) |
| LPE(O) | Deuterium labelled D5- | Deuterium labelled DS- | Unlabeled C18:1 LPE(O)-24.8% |
| | C16:0-LPE(O) in DMEM | C16:0-LPE(O) in DMEM | (w/w) |
| | | | Unlabeled C18:0 LPE(O)-42.4% |
| | | | (w/w) |
| | | | Unlabeled C16:0 LPE(O)- |
| | | | 11.35% (w/w) |
| | with 1% sodium pyruvate | with 2% bovine serum | Deuterium labelled D5-C16:0 |
| | | albumin | LPE(O)-21.45% (w/w) |

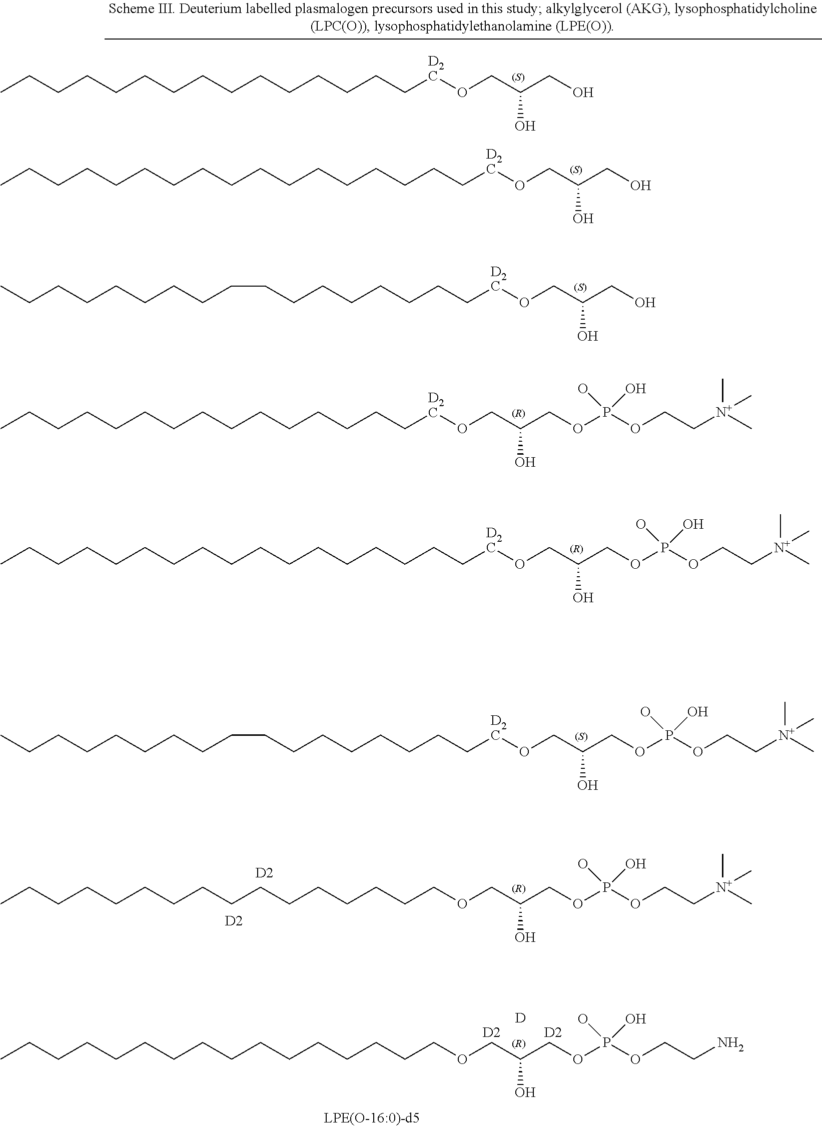

### Cell Culture

Cell lines were selected based on tissues commonly affected in diseases associated with low plasmalogen levels. The two cell lines were the immortalised human hepatoma HepG2 and immortalised mouse fibroblast 3T3. Cells were cultured at 37° C. with 5% CO2 in Dulbecco Modified Eagle's Medium (DMEM) (Gibco, USA) supplemented with 1% sodium pyruvate (Gibco, USA) and either 10% foetal bovine serum (Gibco, USA) or 10% newborn calf serum (Gibco, New Zealand) for HepG2 and 3T3, respectively. HepG2 cells were passaged every week and had their media replenished every two to three days. After passage 30, these cells were discarded. 3T3 cells were passaged every two to three days and discarded after passage 39. Both cell lines were cultured for at least two passages before seeding occurred. HepG2 cells were seeded in a 12-well plate at 300,000 cells/ml of media to achieve 80% confluency, while 3T3 cells reached 100% confluency before experimentation.

### Treatment

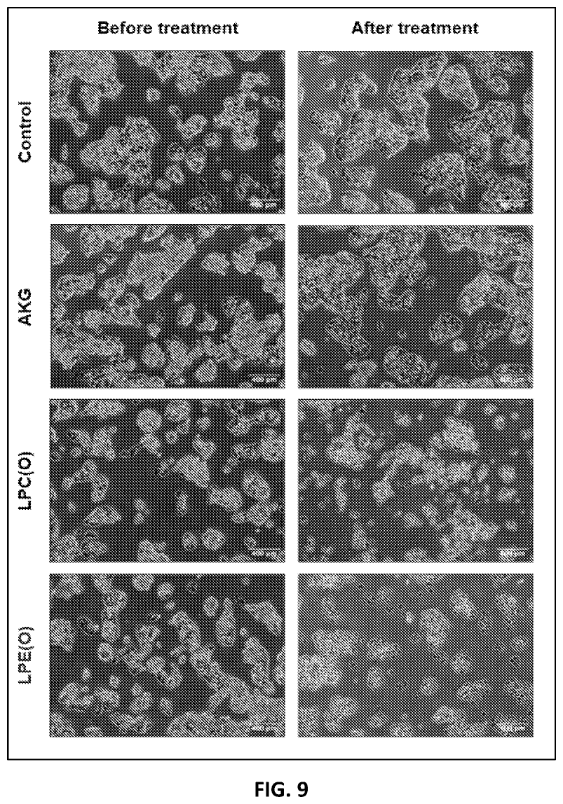

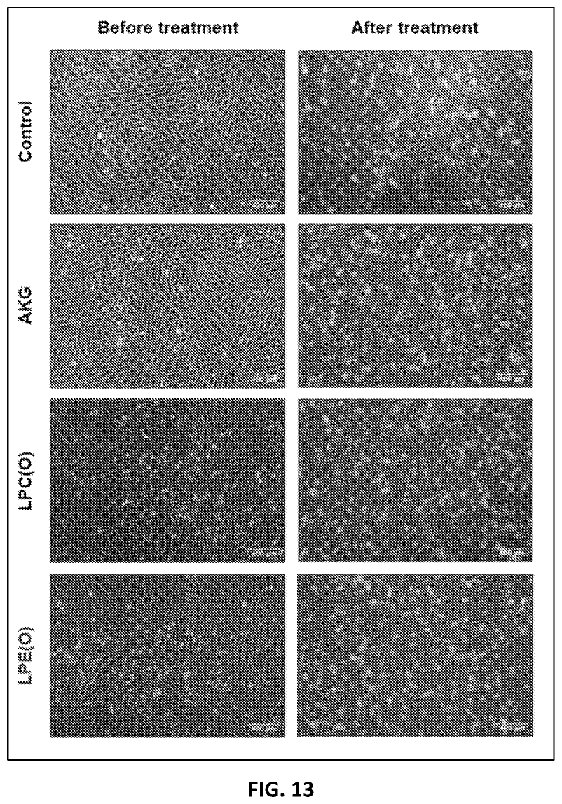

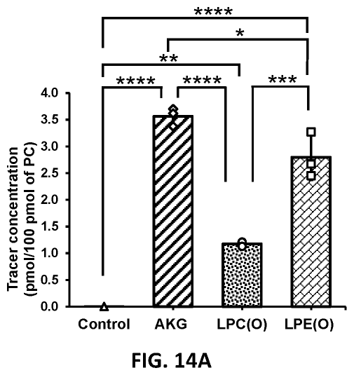

To compare the uptake and incorporation of plasmalogen precursors into plasmalogens, cells were treated with deuterium labelled AKG, LPC(O) and LPE(O) at a concentration of 20 μM (Table 9). As AKG is dissolved in ethanol, the control treatment was media with 0.05% ethanol. Before treatment, images were obtained using an Olympus CKX41 Inverted Microscope, and cells were washed with phosphate-buffered saline (PBS) without Ca²⁺ and Mg²⁺. FIG. 9 and FIG. 13 show the before and after images of HEPG2 cells and 3T3 cells. Cells were then treated with AKG, LPC(O), LPE(O) or control and incubated for 24 hours. After incubation, images were taken and only the adherent cells were harvested. For HepG2, cells were incubated with trypsin-ethylenediaminetetraacetic acid (EDTA) (0.05%), phenol red (Gibco, USA) for 5 minutes at 37° C. and 5% CO2. This was followed by centrifugation for 15 minutes at 13,000 g. The supernatant was then removed, and the remaining pellet was dried using the Speedy Vac and Pump (Thermo Scientific). For 3T3, cells were washed with PBS, and a cell scrapper was used. The cell suspension was then dried using the Speedy Vac and Pump. After the drying process, the samples were stored at −80° C. until lipid extraction was performed.

### Animal Experimentation

To compare the bioavailability and incorporation of plasmalogen precursors into plasmalogen, mice were administered with a deuterium labelled AKG mix, LPC(O) mix, and LPE(O) (Table 9). Lecithin was used as a vehicle to facilitate the oral delivery and as the control treatment. Animal care and experimentation were approved by the Alfred Health and Education Precinct Animal Ethics Committee (P8490). This study involved 64 approximately eight-week-old C57BL/6 mice, consisting of 32 males and 32 females. FIG. 9 shows the dosing schedule for 64 eight-week-old C57BL/6 mice were treated with a single dose of deuterium labelled precursors and control by oral gavage. Precursors include alkylglycerol (AKG), lysoalkylphosphatidylcholine (LPC(O)), lysoalkylphosphatidylethanolamine (LPE(O)). Blood samples were collected by tail-tip bleeding at 0, 1, 4, 24 and 48 hours and through cardiac puncture. Mice were housed at the Precinct Animal Centre of the Baker Heart and Diabetes Institute, with a 12-hour light and dark cycle and unrestricted access to a standard chow diet (SF00-105) and water. There were eight treatment groups, each consisting of four females and four males, which received either a single dose of dose mix A or dose mix B of lecithin (vehicle control), AKG, LPC(O) and LPE(O) (Table 10). Before treatment, mice were randomised into the treatment groups based on body weight and fasted for 4 hours. Each mouse received 200 d of their respective treatment through oral gavage. Blood was collected through tail-tip bleeding before treatment and at 1, 4, 24, and 48 hours after treatment. At the end of the experiment, mice were anesthetised through intraperitoneal injection of sodium pentobarbitone, and blood was collected via cardiac puncture. Mice were then euthanised by cervical dislocation, and tissues were collected and snap-frozen. Collected blood was placed into EDTA tubes and stored on ice. Plasma was separated from the blood via centrifugation for 15 minutes at 1,710 g and 20° C. The plasma supernatant was stored at −80° C. until lipid extraction was conducted.

Table 10. Dose mixes of plasmalogen precursor and control treatments. Precursors include alkylglycerol (AKG), lysoalkylphosphatidylcholine (LPC(O)), lysoalkylphosphatidylethanolamine (LPE(O)).

TABLE 10

| | Dose mix A | Dose mix B |
|---|---|---|
| Compound group | (mg/mouse) | (mg/mouse) |
| Lecithin | 1.0 | 3.3 |
| (vehicle control) | | |
| AKG | 1.0 | 3.3 |
| LPC(O) | 1.5 | 5.0 |
| LPE(O) | 1.4 | 4.5 |

### Lipid Extraction

Lipid extraction was performed to collect the lipids from the cell and plasma samples for lipidomic analysis. As part of the extraction process, Blanks and plasma quality control samples (PQC) pooled from the plasma of healthy individuals were extracted every 10-20 samples. Additionally, the extraction for the mice plasma contained NIST quality control (NIST QC) and mouse plasma quality control (mouse PQC) using plasma from two females and two males from each group. These quality control samples help identify any variation during the extraction process. Depending on the sample type, lipids were extracted using chloroform:methanol (2:1) or butanol:methanol (BUME, 1:1) based on a previously reported method (Alshehry Z. H. et al, Metabolites, 2015 Jun. 17; 5(2):389-403; Meikle P J, Wong G, Tsorotes D, Barlow C K, Weir J M, Christopher M J, et al., “Plasma lipidomic analysis of stable and unstable coronary artery disease,” Arteriosclerosis, Thrombosis, and Vascular Biology.

2011, 31(11):2723-32). In order to calculate the relative lipid concentrations, internal standards that contained >20 non-physiological lipids of known concentration were mixed with either chloroform:methanol or BUME before its use in the extraction.

The extraction for HepG2 cells involved taking 10 μl of sample, Blank and PQC and combining it with 200 μl of chloroform:methanol mixed with internal standards. Samples were then spun on a rotary mixer for 10 minutes, water bath sonicated for 30 minutes and rested at room temperature for 20 minutes. Afterwards, samples were centrifuged for 10 minutes at 13,000 rpm and dried using the Speedy Vac and Pump. Extracted lipids were then reconstituted with 50 μl of water-saturated butanol and sonicated for 10 minutes before adding 50 μl of methanol. Lastly, samples were centrifuged for 5 minutes at 4,000 rpm, and the supernatant was transferred into glass vials (Agilent) with Teflon glass inserts (Agilent) and stored at −80° C. for lipidomic analysis.

The extraction for 3T3 cells involved taking 10 μl of sample, Blank and PQC and combining it with 100 μl of BUME mixed with internal standards. Samples were then vortexed for 10 seconds, water bath sonicated for 60 minutes and centrifuged for 10 minutes at 13,000 rpm. The supernatant was then transferred into glass vials with Teflon glass inserts for lipidomic analysis.

The mice plasma extraction also used BUME but with only 5 μl of a sample mixed with 5 μl of water. Samples included the plasma supernatant, Blanks, PQCs, NIST QCs, and mouse PQCs. Since only 5 μl of a sample was used, half of the concentration of internal standards was mixed with BUME compared to the extraction of HepG2 and 3T3 cells.

To extract the lipids from brain samples, approximately 50 mg of brain tissues were homogenised in 500 μL of ice-cold phosphate buffered saline using a TissueLyser II (Qiagen, USA) for 60 s and then sonicated with a Misonix S-4000 Sonicator (Thermo Fisher Scientific, Melbourne, VIC, Australia) for 10-15 s at amplitude 25. Protein content of the homogenates was quantified using a Pierce™ BCA protein assay kit (Thermo Fisher Scientific, Rockford, IL, USA). Homogenates were then made up to a stock protein concentration of 5 mg/mL protein and 10 μL aliquots from the stock solutions, containing 50 μg of protein, were subsequently used for lipid extraction. Lipids were extracted following single-phase chloroform:methanol (2:1) extraction procedure as described previously (Meikle P J, Wong G, Tsorotes D, Barlow C K, Weir J M, Christopher M J, et al., “Plasma lipidomic analysis of stable and unstable coronary artery disease,” Arteriosclerosis, Thrombosis, and Vascular Biology. 2011, 31(11):2723-32). Lipid extractions were performed as a single batch with quality control samples (pooled plasma QC, pooled tissue QC, NIST QCs, and blanks) included approximately every 10 samples.

### Lipidomic Analysis by UHPLC/MS/MS

A targeted lipidomic analysis was conducted on the lipid extracts of HepG2 cells, 3T3 cells and plasma samples using-high-performance liquid chromatography-electrospray ionization-tandem mass spectrometry (UHPLC/MS/MS). Before analysis, lipid extracts were randomised, thawed at room temperature for an hour and water bath sonicated for 15 minutes. Lipids were quantified using an Agilent 1290 series UHPLC system combined with an Agilent 6495C triple quadrupole mass spectrometer. First, samples were placed in the autosampler tray at 20° C. and either 1 μl of cell sample or 2 μl of plasma sample was injected. Lipids were separated with a stepped linear gradient of solvent A and solvent B under the liquid chromatography conditions outlined in Table 11 and Table 12. Lipids were then identified based on their retention time, precursor ion and product ion masses under the mass spectrometry conditions outlined in Table 13.

Table 11. Ultra-high-performance liquid chromatography conditions for lipidomic analysis.

TABLE 11

| Conditions | |
|---|---|
| Solvent A | 50% water, 30% acetonitrile, 20% isopropanol, |
| | 10 mM ammonium formate |
| Solvent B | 1% water, 9% acetonitrile, 90% isopropanol, |
| | 10 mM ammonium formate |
| Column | ZORBAX eclipse plus C18 column: |
| | 2.1 × 100 mm, 1.8 mm, Agilent |
| Column temperature | 45° C. |
| Flow rate | 0.4 ml/min |

Table 12. Ultra-high-performance liquid chromatography gradient conditions.

TABLE 12

| Time (minutes) | % of Solvent A | % of Solvent B |
|---|---|---|
| 0.0 | 85.0 | 15.0 |
| 2.5 | 50.0 | 50.0 |
| 2.6 | 43.0 | 57.0 |
| 9.0 | 30.0 | 70.0 |
| 9.1 | 7.0 | 93.0 |
| 11.0 | 4.0 | 96.0 |
| 11.1 | 0.0 | 100.0 |
| 12.0 | 0.0 | 100.0 |
| 12.2 | 85.0 | 15.0 |
| 16.0 | 85.0 | 15.0 |

Table 13. Mass spectrometry conditions for lipidomic analysis.

TABLE 13

| Conditions | |
|---|---|
| Gas temperature | 150° C. |
| Gas flow rate | 171/min |
| Sheath gas temperature | 200° C. |
| Sheath gas flow | 101/min |
| Capillary voltage | 3500 V |
| Nebulizer | 20 psi |

A modified version of a previously reported MRM method was used to detect deuterium incorporation into lipid species of the glycerophospholipids and glycerolipids classes (Huynh, K. et al., 2019) Both the deuterated and endogenous non-labelled versions of lipids were measured. The lipids with the deuterated isotope are denoted by [+1], [+2], [+4] and [+5], whereas the endogenous lipids are referred to as [+0]. Precursors labelled with D4 produce [+4] lipid species, while those labelled with D5 result in [+5] lipid species. However, precursors labelled with D2 produce both [+1] and [+2] lipid species due to the location of the deuteriums, making them susceptible to removal by the PEDS1 enzyme when producing plasmalogen. The precursor mass of the lipid species labelled with D4 was offset by 4 Da, as one deuterium is equivalent to 1 Da. The product ions were offset based on lipid class fragmentation patterns.

The Agilent MassHunter Quantitative Analysis v10.0 software was used to integrate the peaks and quantify the area under the curve of each lipid. To calculate the relative concentration of the deuterium labelled lipids in the cell samples, we first subtracted the background signal area from the Blank samples. This removes any unrelated background signal that the mass spectrometer has identified. Next, we calculated the isotope ratio in the control samples, which corresponds to the area of deuterium labelled lipids over the area of the endogenous lipids. This calculates the natural isotope of each lipid that should be removed. We then multiplied the isotope ratio by the subtracted background area for each sample to get the endogenous signal for each lipid. This value was then subtracted from all the deuterium labelled lipid areas. Afterwards, we divided this value by the area of the internal standard of the corresponding lipid class and multiplied it by the amount of internal standard added. This provided the relative concentration of the deuterium lipid in pmol/sample. Any variation in lipid concentrations among samples, such as differences in cell numbers, were removed by normalising to one of the most abundant phospholipids, PC 34:1. The relative concentration of PC 34:1 was calculated by dividing the area of PC 34:1 by the area of its internal standard. This was then multiplied by 100 to give a concentration of μmol/sample. The relative concentration of the deuterium lipid was then divided by the relative concentration of PC 34:1 to provide a concentration of pmol/μmol of PC 34:1. The relative concentrations of each lipid class were also calculated as the sum of the individual species within the class. When calculating the sum of TG(O), only the neutral loss species were considered.

The relative lipid concentrations in the mice were calculated with an additional step of multiplying the deuterium lipid concentration (μmol/sample) by 200 to give a concentration of pmol/ml of plasma before normalising to PC 34:1. Furthermore, for the calculations of LPE(O), the concentration was divided by 0.65 since there were both labelled and unlabeled precursors.

### Effect of Plasmalogen Precursors on the Plasmalogen Biosynthesis Pathway in HepG2 Cells

To compare the uptake and incorporation of plasmalogen precursors into plasmalogens, HepG2 cells were treated with deuterium labelled plasmalogen precursors at 20 μM for 24 hours, and lipid concentrations were analysed using LC-MS/MS. Images taken before and after treatment revealed that LPC(O) and LPE(O) led to floating cells and smaller adherent cells, whereas AKG did not affect cell morphology compared to the control (FIG. 9).

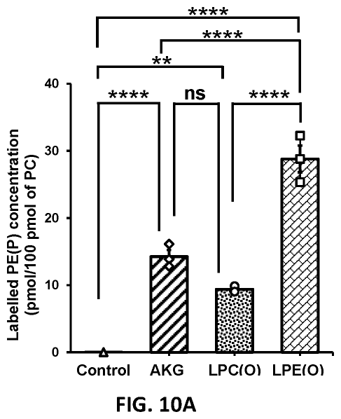

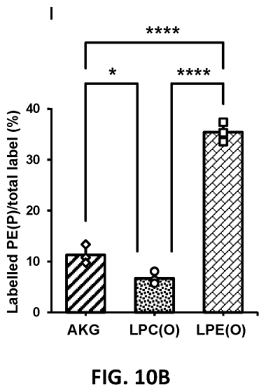

The levels of labelled PE(P) of the 16:0, 18:0 and 18:1 species were compared between treatment groups. It was found that concentrations were significantly higher in the plasmalogen precursor groups compared to the control (p≤0.01, FIG. 10A). This increase in labelled PE(P) was most evident in LPE(O), followed by LPC(O) and then AKG (FIG. 10A). To better understand how efficiently the precursor is metabolised into plasmalogen, the ratio of labelled PE(P) concentration to the total label concentration was calculated. Here, total label refers to the sum of labelled AKG, alkyl-acylglycerol (DG(O)), LPC(O), lysophosphatidylcholine plasmalogen (LPC(P)), LPE(O), lysophosphatidylethanolamine plasmalogen (LPE(P)), alkylphosphatidylcholine (PC(O)), phosphatidylcholine plasmalogen (PC(P)), alkylphosphatidylethanolamine (PE(O)), PE(P), monoalkyl-diacylglycerol (TG(O)). This approach is a better representation of the efficiency of the precursor since it accounts for the amount of precursor taken up by the cell. Whilst some precursors may have a greater uptake and result in a higher production of PE(P), this does not necessarily mean that it is more efficient in producing PE(P) if it requires more of the precursor. Comparisons between plasmalogen precursor groups showed that the ratio of LPE(O) is significantly greater than both AKG and LPC(O) (p≤0.0001, FIG. 10B). Additionally, the ratio of AKG is significantly increased compared to LPC(O) (p≤0.05, FIG. 10B). However, the difference between AKG and LPC(O) is smaller than the difference between LPE(O) and other precursors.

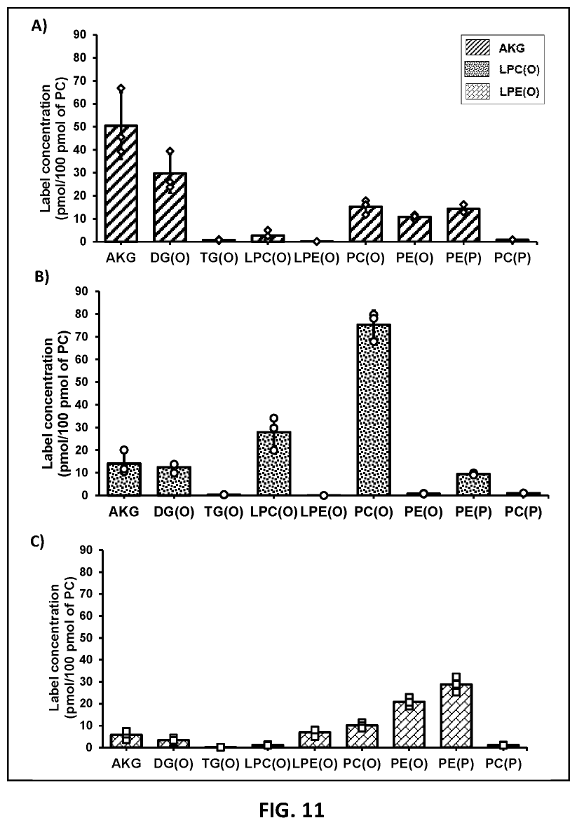

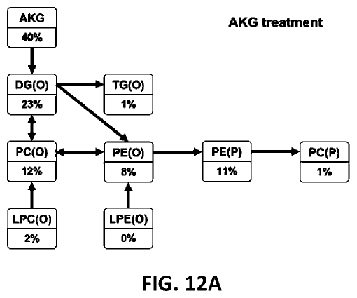

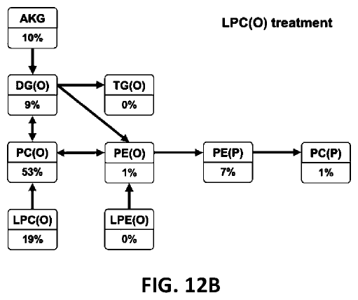

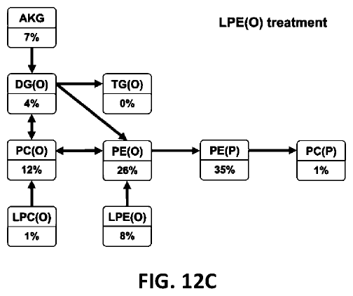

Bar charts were created to visualise the distribution of the label among lipid classes of the plasmalogen biosynthesis pathway (FIG. 11). Following AKG treatment, a large proportion of label was incorporated into AKG and DG(O), referring to 40% and 23% of the total label, respectively (FIG. 11A and FIG. 12A). In comparison, there was minimal incorporation into TG(O), LPC(O), LPE(O) and PC(P) and moderate incorporation into PC(O), PE(O) and PE(P), where PE(P) contained 11% of the total label (FIG. 11A and FIG. 12A). Following LPC(O) treatment, there was high incorporation into LPC(O) and greater incorporation into PC(O), which represented 53% of the total label (FIG. 11B and FIG. 12B). There was moderate incorporation into AKG, DG(O) and PE(P), where PE(P) consisted of 7% of the total label (FIG. 11B and FIG. 12B). The remaining lipid classes showed little to no incorporation (FIG. 11B). Following LPE(O) treatment, there was moderate incorporation into AKG, LPE(O) and PC(O) and higher incorporation into PE(O) and PE(P) (FIG. 11C). Most of the label was incorporated into PE(P) and represented 35% of the total label (FIG. 12C). Otherwise, there was low incorporation into DG(O) and minimal incorporation into TG(O), LPC(O) and PC(P) (FIG. 11C).

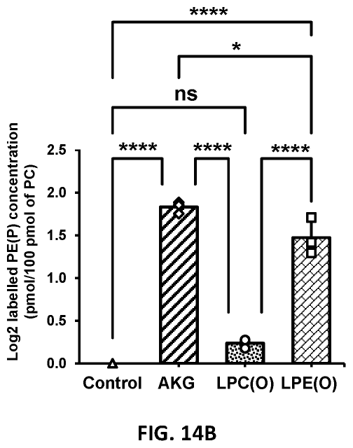

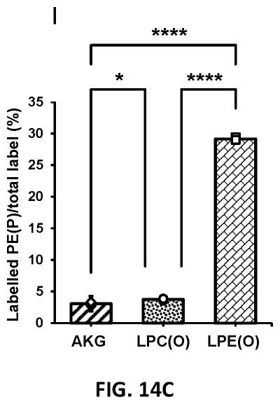

To validate the findings of the HepG2 cells, 3T3 cells were treated with deuterium labelled plasmalogen precursors at 20 μM for 24 hours, and lipid concentrations were analysed using LC-MS/MS. Unlike HepG2 cells, plasmalogen precursor treatment in 3T3 cells led to no changes in cell morphology compared to control (FIG. 13). By comparing labelled PE(P) concentrations between treatment groups, it was found that concentrations were significantly increased following plasmalogen precursor treatment compared to control (p≤0.01, FIG. 14A and FIG. 14B). This increase was most noticeable in AKG, followed by LPE(O) and then LPC(O) (FIG. 14A). However, when comparing the ratio of labelled PE(P) concentration to the total label concentration, it was observed that LPE(O) was significantly higher than both AKG and LPC(O), which is consistent with the results from the HepG2 cells (FIG. 14C). Although LPC(O) had a higher ratio than AKG, this difference was considerably smaller than that between LPE(O) and AKG (FIG. 14C).

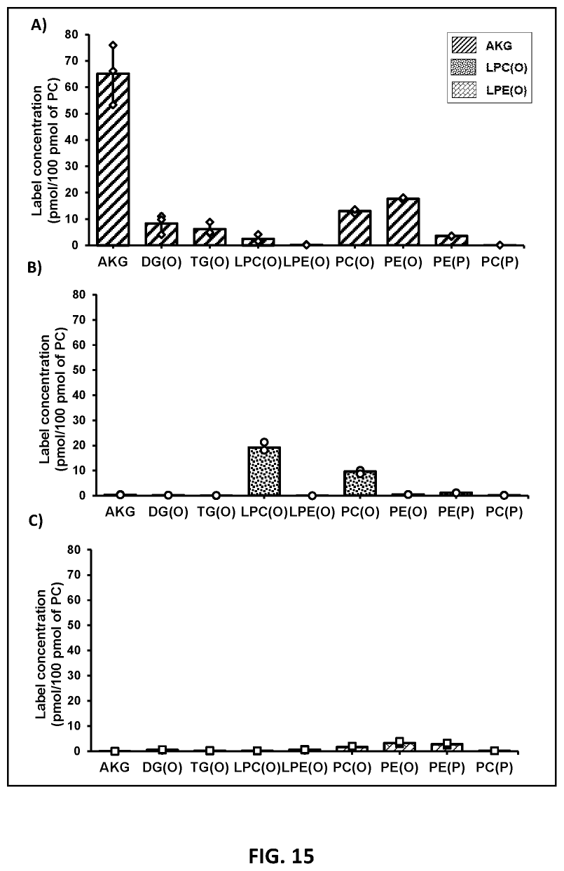

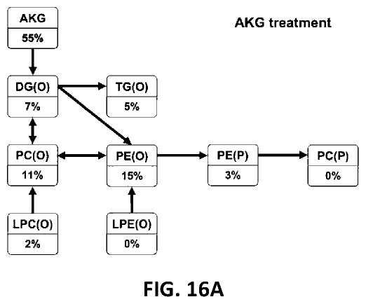

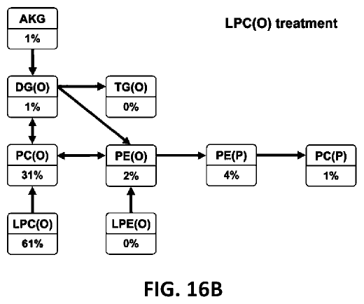

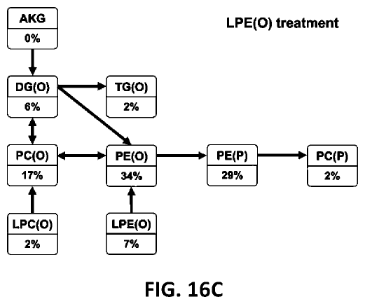

Bar charts were similarly generated to evaluate the label distribution within the plasmalogen biosynthesis pathway (FIG. 15). Following AKG treatment, a high proportion of the label was incorporated into AKG, representing 55% of the total label (FIG. 15A and FIG. 16A). This finding was consistent with the results observed in HepG2 cells. In addition, there was moderate incorporation in PC(O) and PE(O) and minimal incorporation into the remaining lipid classes, with only 11% of the total label being incorporated into PE(P) (FIG. 15A and FIG. 16A). When comparing the effects of AKG treatment between cell lines, it was found that a lower proportion of label was incorporated into PE(P) and DG(O) in 3T3 cells compared to HepG2 cells (FIG. 11A and FIG. 15A). Following LPC(O) treatment, there was high incorporation into LPC(O) and moderate incorporation into PC(O), which is opposite to the results observed in HepG2 cells (FIG. 11B and FIG. 15B). There was little to no incorporation into the remaining lipid classes, with only 4% of the total label being incorporated into PE(P) (FIG. 15B and FIG. 16B). Following LPE(O) treatment, there was moderate incorporation into LPE(O) and higher incorporation into PC(O), PE(O) and PE(P) (FIG. 15C). While a large proportion of the label was incorporated into PE(P) in HepG2 cells, most of it was incorporated into PE(O) in 3T3 cells and accounted for 34% of the total label (FIG. 12C and FIG. 16C). There was also a high incorporation into PE(P), representing 29% of the total label (FIG. 16C). The rest of the lipid classes showed little to moderate incorporation (FIG. 15C).

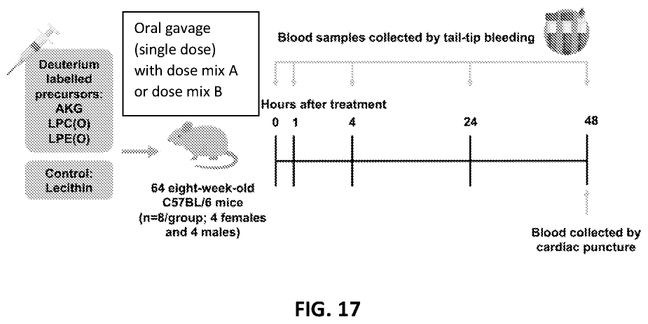

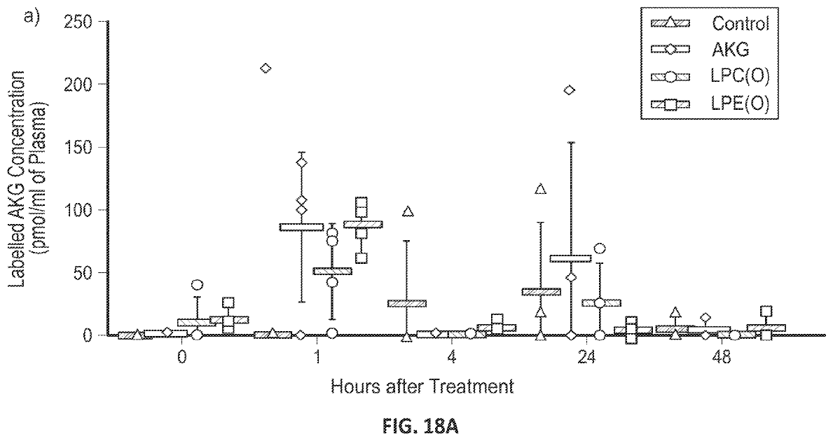

To compare the bioavailability and incorporation of plasmalogen precursors into plasmalogens, mice were treated with a single dose of deuterium labelled plasmalogen precursors (FIG. 17). The plasma lipid concentrations from multiple time points were analysed using LC-MS/MS. Unlike the cell lines, lipidomic analysis in the mice considered only the 16:0 lipid species, as LPE(O) treatment only used labelled 16:0 compared to AKG and LPC(O), which had 16:0, 18:0 and 18:1. Although all treated mice had their lipid concentrations measured, the data reported here mainly focuses on dose mix A treated mice since dose mix B treated mice exhibited similar trends.

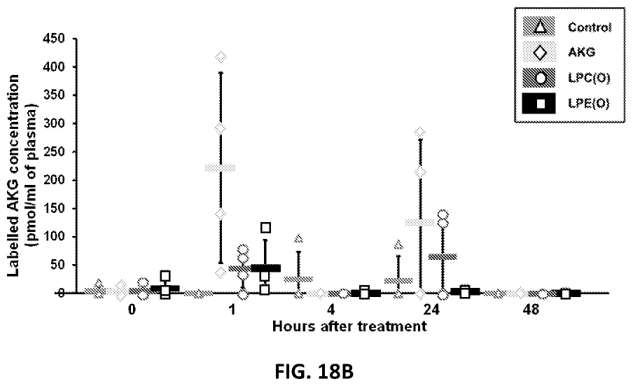

The levels of labelled AKG were compared between treatment groups to estimate the rate at which AKG is taken up into circulation. In females, there is a high concentration of labelled AKG for all precursors at 1 hour after treatment (FIGS. 18A, 19A, 20A). However, in males, AKG treatment led to a more profound increase in labelled AKG after 1 hour of treatment compared to LPC(O) and LPE(O) (FIGS. 18B, 19B and 20B). Furthermore, AKG treatment in males resulted in approximately twice the amount of labelled AKG compared to females (FIG. 18A and FIG. 18B).

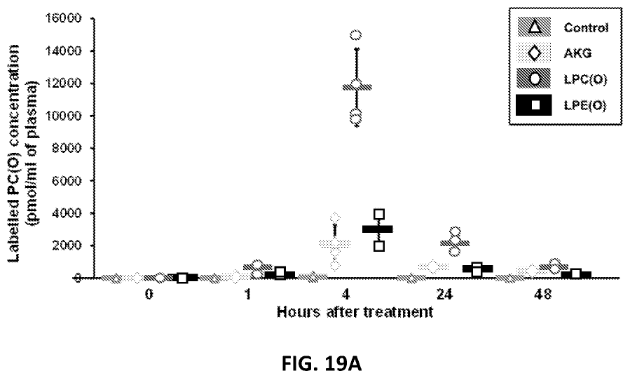

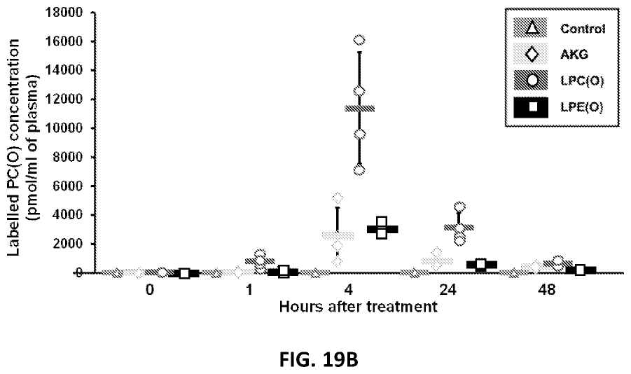

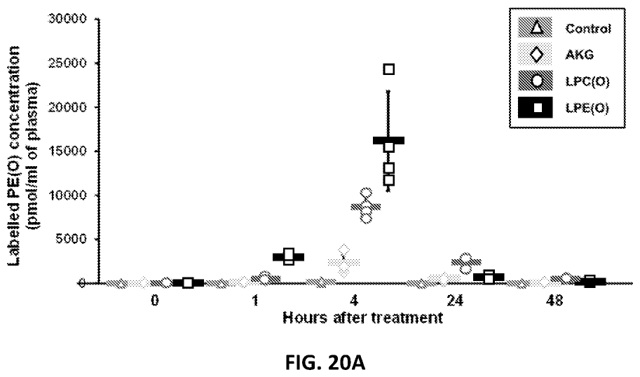

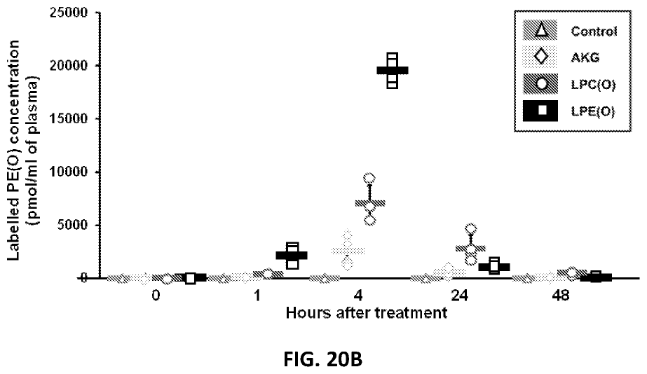

The levels of labelled PC(O) and PE(O) were measured since plasmalogen precursors are initially converted into these lipids before producing plasmalogen. For all plasmalogen precursors, the highest concentration of labelled PC(O) occurred 4 hours after treatment (FIG. 19A and FIG. 19B). Moreover, treatment with LPC(O) resulted in the highest increase in labelled PC(O), followed by LPE(O), and then AKG (FIG. 19A and FIG. 19B). Similar to PC(O), labelled PE(O) concentration was at its highest after 4 hours of treatment for all plasmalogen precursors (FIG. 20A and FIG. 20B). LPE(O) treatment produced the highest increase in labelled PE(O), followed by LPC(O), and then AKG.

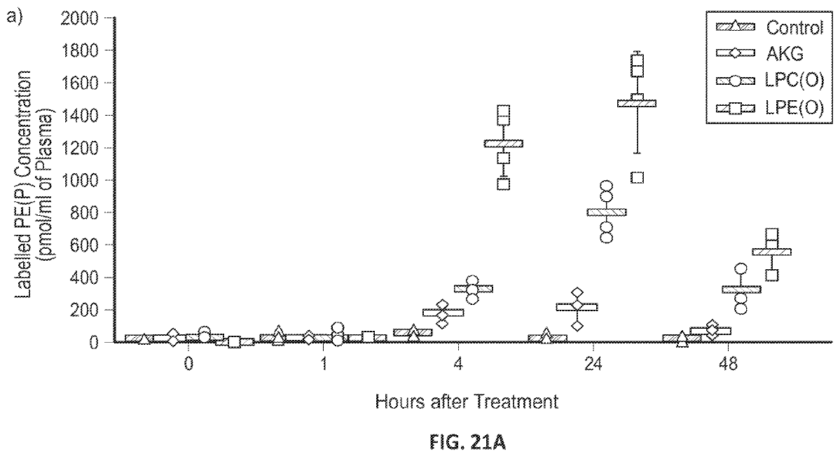

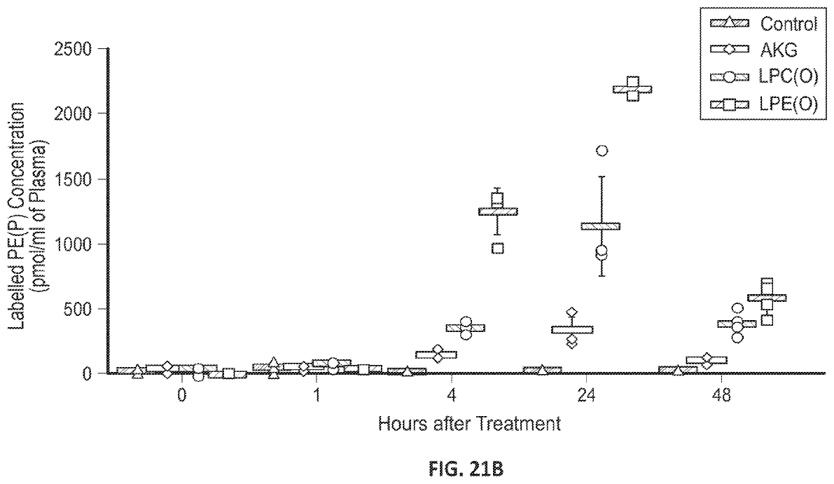

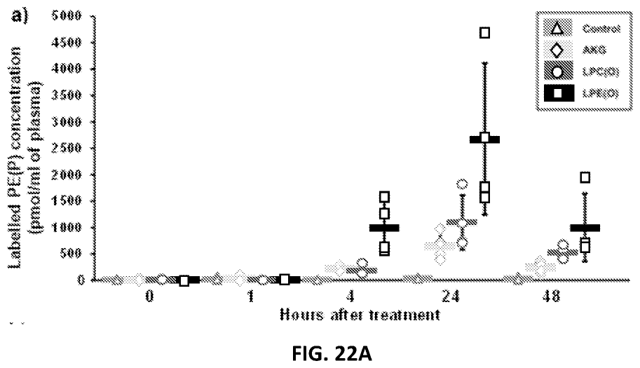

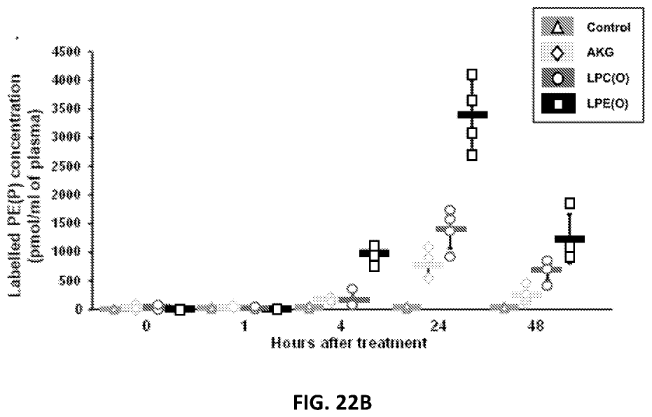

When examining labelled PE(P) concentration, it was revealed that LPE(O) treatment resulted in the highest increase, followed by LPC(O), and then AKG (FIG. 21A, FIG. 21B, FIG. 22A, FIG. 22B). This was observed after 24 hours of treatment and was consistent in females and males who received either dose mix A (FIG. 21A, FIG. 21B) or dose mix B (FIG. 22A, FIG. 22B) treatment dose.

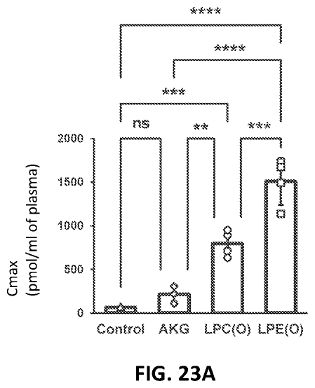

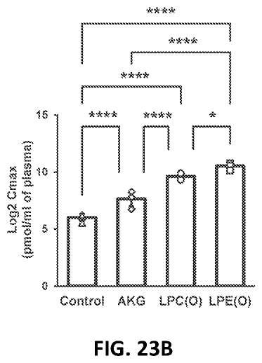

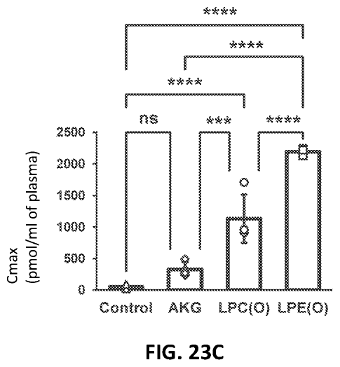

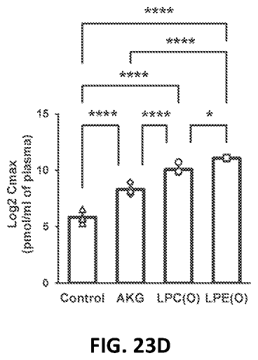

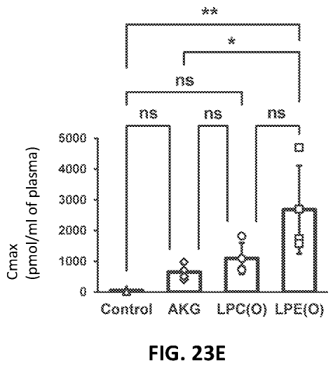

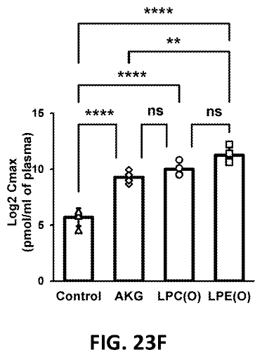

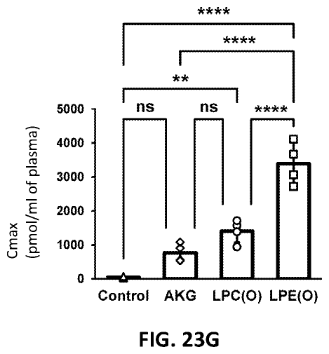

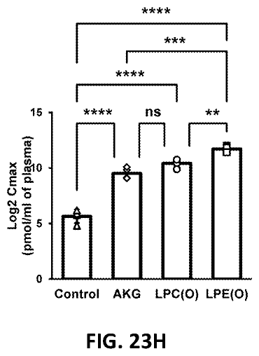

The Cₘₐₓ of PE(P) was compared between treatment groups to evaluate the bioavailability of plasmalogen precursors. In both males and females treated with dose mix A, LPC(O) and LPE(O) treatment showed a significantly higher Cₘₐₓ than the control and AKG (p≤0.01, FIG. 23A, FIG. 23B and FIG. 23C, FIG. 23D). Furthermore, LPE(O) treatment led to a significantly higher Cₘₐₓ compared to LPC(O) (p≤0.001, FIG. 23A, and FIG. 23C). Males treated with LPE(O) treatment produced 1.45× higher Cₘₐₓ than females FIG. 23A and FIG. 23C). Similar trends were evident in mice treated with a dose mix B (FIG. 23E, FIG. 23F and FIG. 23G, FIG. 23H). In both males and females, LPE(O) treatment resulted in a significantly increased Cₘₐₓ compared to control and AKG (p≤0.05, FIG. 23E and FIG. 23G). Additionally, LPE(O) treatment led to a significantly larger Cₘₐₓ than LPC(O) in males, while this difference was only observed as a trend in females (p≤0.0001, FIG. FIG. 23E and FIG. 23G). Treatment with LPC(O) produced a greater Cₘₐₓ than AKG but was only seen as a trend in the dose mix B treated mice compared to the dose mix A treated mice (FIGS. 23A-23H). When comparing mice treated with different doses, it was found that a dose mix B of LPE(O) led to 1.77× higher Cₘₐₓ than a dose mix A in females (FIG. 23A and FIG. 23E). In comparison, a dose mix B of LPE(O) led to 1.55× greater Cₘₐₓ than a dose mix A in males (FIG. 23C and FIG. 23G).

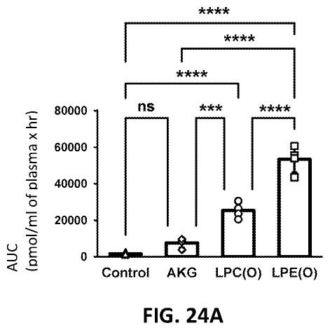

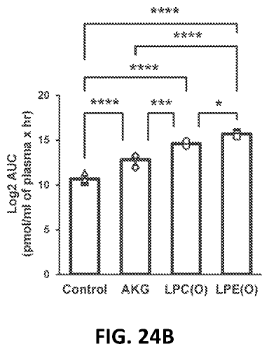

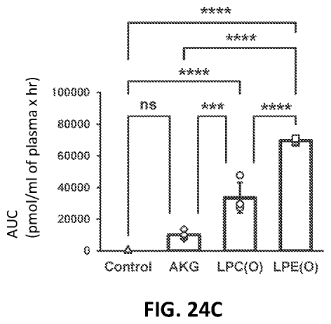

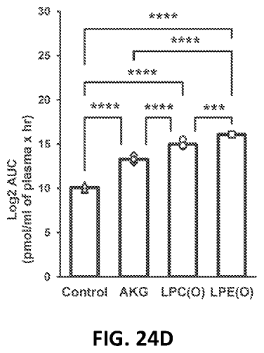

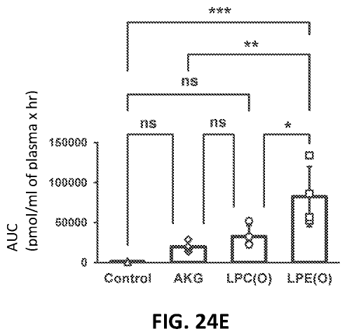

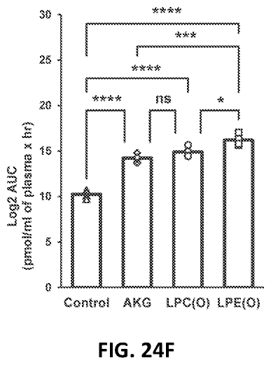

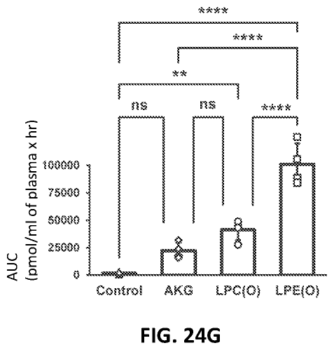

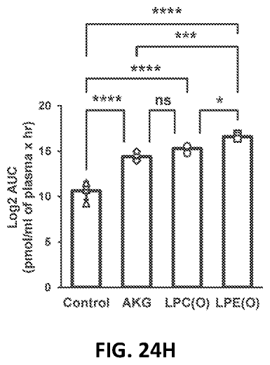

The bioavailability of plasmalogen precursors was further evaluated by comparing the AUC of PE(P) between the groups. Similar to the Cₙₐₓ results, both males and females treated with dose mix A of LPC(O) or LPE(O) showed a significantly higher AUC than control or AKG (p≤0.001, FIG. 24A, FIG. 24B and FIG. 24C and FIG. 24D). In addition, LPE(O) treatment produced a significantly greater AUC than LPC(O) (p≤0.0001, FIG. 24A and FIG. 24C). It was further observed that LPE(O) treatment in males resulted in 1.3× higher AUC than in females (FIG. 24A and FIG. 24C). Likewise, these trends were reflected in mice treated with a dose mix B. LPE(O) treatment resulted in a higher AUC than LPC(O), AKG or control in both males and females (p≤0.05, FIG. 24E, FIG. 24F and FIG. 24G and FIG. 24H). LPC(O) treatment resulted in a greater AUC than AKG (FIG. 24E and FIG. 24G). When comparing mice treated with different doses, it was observed that a dose mix B of LPE(O) produced a 1.5× higher AUC than a dose mix A in both females and males (FIGS. 24A-24H).

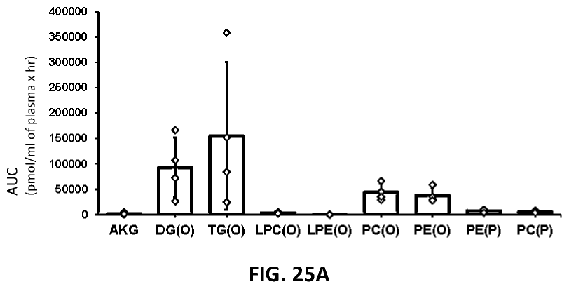

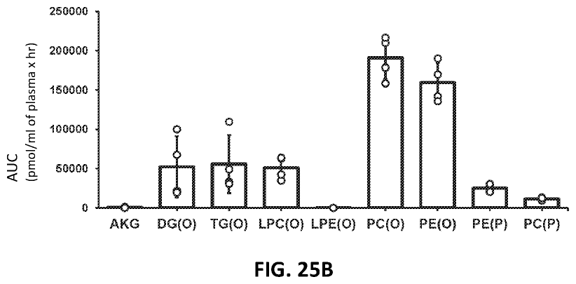

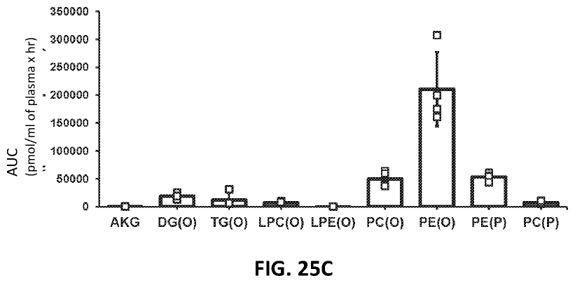

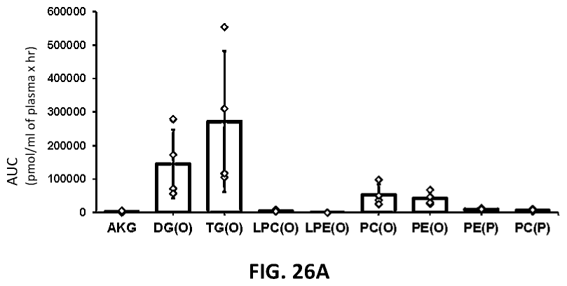

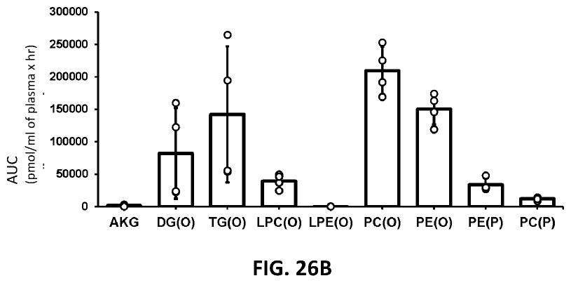

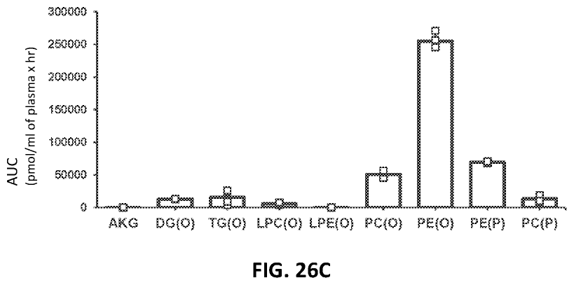

Bar charts of AUCs for females and males treated with a dose mix A were created to visualise the distribution of the label across the plasmalogen biosynthesis pathway (FIG. 25 and FIG. 26). In females, AKG treatment resulted in high incorporation into DG(O) and TG(O) and moderate incorporation into PC(O) and PE(O) (FIG. 25A). There was also minimal to no incorporation into AKG, LPC(O), LPE(O), PE(P) and PC(P) (FIG. 25A). Similar trends were evident after AKG treatment in males (FIG. 26A). In comparison, LPC(O) treatment in females led to moderate incorporation into DG(O), TG(O), LPC(O) and PE(P) and high incorporation into PC(O) and PE(O) (FIG. 25B). Little to no incorporation was shown in AKG, LPE(O) and PC(P) (FIG. 25B). Whilst LPC(O) treatment led to a greater increase in TG(O) in males, the other lipid classes exhibited similar trends to females (FIG. 25B and FIG. 26B). Following LPE(O) treatment in both females and males, there was high incorporation into PE(O), moderate incorporation into PC(O) and PE(P) and little to no incorporation into the remaining lipid classes (FIG. 25C and FIG. 26C).

### Tissue Analysis

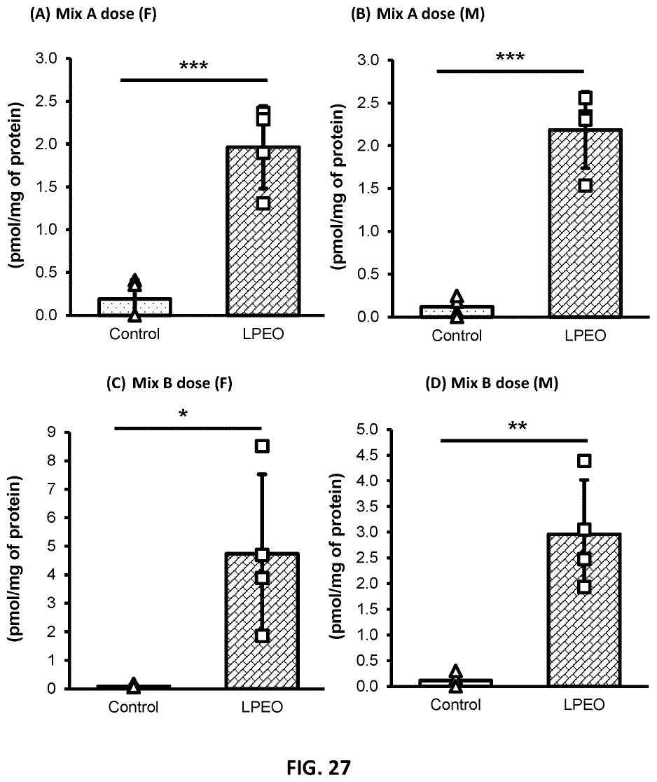

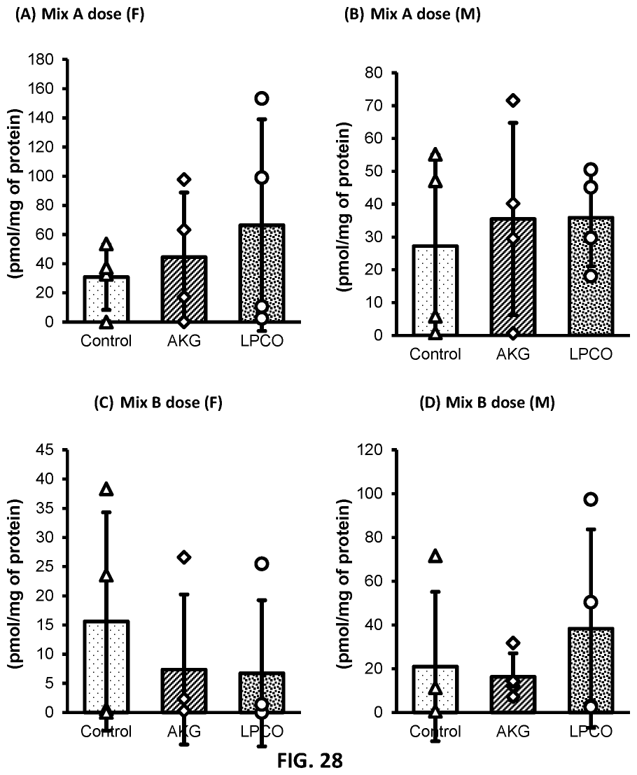

The conversion of labelled precursor compounds into brain PE plasmalogens was tested and it was observed that LPE(O) supplementation led to small but significant increases in labelled PE plasmalogens in the brain (FIG. 27), whereas the increases in labelled PE(P) with AKG and LPC(O) supplementation were quite variable (FIG. 28).

### Statistical Analysis

Lipidomic analysis was performed on triplicates of HepG2 and 3T3 cells and four replicates for the plasma samples. However, samples collected from males treated with a dose mix A of lecithin had five replicates at 0 hours after treatment. This is because one mouse had died before treatment but was still included in the analysis. Figures were produced using Microsoft Excel and presented as mean±standard deviation. Statistical analysis was conducted using GraphPad Prism 9.5.1. A one-way analysis of variance (ANOVA) was performed to compare treatment groups, followed by Tukey's post-hoc test to determine which groups were significantly different. Significance was indicated by * p≤0.05, ** p≤0.01, *** p≤0.001 and **** p≤0.0001. p<0.05 was considered statistically significant.

### Example 5

This example relates to the modulation of plasmalogen level and composition by LPC(O) and LPE(O) supplementation in RAW 264.7 cells using different SN-1 alkyl compositions.

### Cell Culture Methods

A mouse macrophage cell line, RAW 264.7 was used for this study. The cells were cultured at 37° C. with 5% CO₂ in Roswell Park Memorial Institute (RPMI) 1640 medium containing L-glutamine with 1% sodium pyruvate (Gibco, USA) and 10% fetal bovine serum (Gibco, USA). The cells were passaged every week and the media replenished every two to three days until passage 30, at which point these cells were discarded. The cells were seeded in a 12-well plate at a concentration of 300,000 cells/ml of media and allowed to grow until they reached 80% confluency.

### Plasmalogen Precursor Supplementation of Cells

To assess the incorporation of plasmalogen precursors into cellular plasmalogens, cells were supplemented with compounds with different SN1 compositions, i.e., varying proportions of O-16:0, O-18:0, and O-18:1 of LPC(O) or LPE(O) at a concentration of 20 μM (Table 14). The plasmalogen precursors, LPC(O), and LPE(O), were dissolved in chloroform:methanol (2:1), evaporated under a stream of nitrogen gas at 40° C., and then reconstituted in RPMI medium with 2% bovine serum albumin (Sigma-Aldrich, USA). The control treatment for the cells consisted of RPMI with 2% bovine serum albumin. Once prepared, the media containing the precursors were vortexed and sonicated in an ultrasonic water bath (Soniclean, Adelaide, SA, Australia) for 15 minutes.

Table 14. List of plasmalogen precursor compounds and their molar % used in this study.

TABLE 14

| Group | |
|---|---|
| Number | Compound group |
| 1 | Vehicle control |
| 2 | LPC(O-16:0) 100% |
| 3 | LPC(O-18:0) 100% |
| 4 | LPC(O-18:1) 100% |
| 5 | LPC(O-16:0) 50% + LPC(O-18:0) 50% |
| 6 | LPC(O-18:0) 50% +LPC(O-18:1) 50% |
| 7 | LPC(O-16:0) 50% + LPC(O-18:1) 50% |
| 8 | LPC(O-16:0) 26.8% + LPC(O-18:0) 46.1% + LPC(O-18:1) |
| | 27.1% |
| 9 | LPC(O-16:0) 46% + LPC(O-18:0) 21% + LPC(O-18:1) 33% |
| 10 | LPC(O-16:0) 42% + LPC(O-18:0) 51% + LPC(O-18:1) 7% |
| 11 | LPC(O-16:0) 62% + LPC(O-18:0) 23.9% + LPC(O-18:1) 14.1% |
| 12 | LPC(O-16:0) 19.8% + LPC(O-18:0) 66% + LPC(O-18:1) 14.2% |
| 13 | LPC(O-16:0) 32.5% + LPC(O-18:0) 39.6% + LPC(O-18:1) |
| | 27.9% |
| 14 | LPC(O-16:0) 34.1% + LPC(O-18:0) 41.5% +LPC(O-18:1) 24.4% |
| 15 | LPE(O-16:0) 100% |
| 16 | LPE(O-18:0) 100% |
| 17 | LPE(O-18:1) 100% |
| 18 | LPE(O-16:0) 50% + LPE(O-18:0) 50% |
| 19 | LPE(O-18:0) 50% + LPE(O-18:1) 50% |
| 20 | LPE(O-16:0) 50% + LPE(O-18:1) 50% |
| 21 | LPE(O-16:0) 26.8% + LPE(O-18:0) 46.1% + LPE(O-18:1) |
| | 27.1% |
| 22 | LPE(O-16:0) 46% + LPE(O-18:0) 21% + LPE(O-18:1) 33% |
| 23 | LPE(O-16:0) 42% + LPE(O-18:0) 51% + LPE(O-18:1) 7% |
| 24 | LPE(O-16:0) 62% + LPE(O-18:0) 23.9% + LPE(O-18:1) 14.1% |
| 25 | LPE(O-16:0) 19.8% + LPE(O-18:0) 66% + LPE(O-18:1) 14.2% |
| 26 | LPE(O-16:0) 32.5% +LPE(O-18:0) 39.6% + LPE(O-18:1) 27.9% |
| 27 | LPE(O-16:0) 34.1% + LPE(O-18:0) 41.5% + LPE(O-18:1) 24.4% |

### Lipid Extraction

Lipids were extracted from cells using the butanol-methanol method, following established protocols. Each lyophilized cell sample received 10 μl of MilliQ water and 100 μl of a butanol-methanol mixture (1:1) containing a specific set of internal standards. After this, each sample was vortexed for several seconds and was placed in a sonicator bath for 1 hour. Subsequently, the samples were centrifuged at 13,000 g for 15 minutes, and the resulting supernatant was carefully transferred into mass spectrometry vials with inserts. These vials were then frozen at −80° C. until they were ready for mass spectrometry analysis.

### Lipidomic Analysis

A targeted lipidomic approach using LC-MS/MS, following a previously published protocol (Huynh, et al., “High-Throughput Plasma Lipidomics: Detailed Mapping of the Associations with Cardiometabolic Risk Factors,” Cell Chem. Biol., 2019 Jan. 17; 26(1):71-84.e4, doi: 10.1016/j.chembiol.2018.10.008), was utilised for the samples from this study. This method employs liquid chromatography (Agilent 1290 Infinity) coupled with tandem mass spectrometry (Agilent 6495C) operating in the dynamic Multiple Reaction Monitoring (MRM) mode. Multiple lipid species belonging to different lipid classes including phosphatidylcholine (PC), alkylphosphatidylcholine [PC(O)], alkenylphosphatidylcholine [PC(P)], phosphatidylethanolamine (PE), alkylphosphatidylethanolamine [PE(O)], alkenylphosphatidylethanolamine [PE(P)] or PE plasmalogen, lysophosphatidylcholine (LPC), lysoalkylphosphatidylcholine [LPC(O)], lysoalkenylphosphatidylcholine [LPC(P)], lysophosphatidylethanolamine (LPE), lysoalkylphosphatidylethanolamine [LPE(O)], lysoalkenylphosphatidylethanolamine [LPE(P)], triacylglycerol (TG) and alkyldiacylglycerol [TG(O)] were measured. The lipid species measured in this study are listed in Supplementary Table 1, which is included at the end of this Example.

The chromatography analytical gradient utilised in this analysis involved a series of steps: starting with a flow rate of 0.4 mL/minute at 15% B, it increased to 50% B over 2.5 minutes, followed by increments to 57% over 0.1 minutes, 70% over 6.4 minutes, 93% over 0.1 minute, and finally to 96% over 1.9 minutes, terminating in 100% B over 0.1 minute. The solvent was held at 100% B for 0.9 minutes, totaling a runtime of 12.0 minutes. Equilibration was initiated by reducing the solvent from 100% B to 15% B over 0.2 minutes and maintained for a total of 16 minutes.

For data processing, vendor software Qualitative Analysis B.07.00 and QQQ Quantitative Analysis B.10.0.0 were utilized. Relative quantification of lipid species was determined by comparing them to the corresponding internal standards. The total concentrations of lipid classes were determined by summing the concentrations of individual lipid species within each respective lipid class.

### Statistical Analysis

Lipidomic analysis was conducted on triplicate samples of cells. Figures were generated using Microsoft Excel and presented as mean values with standard deviation error bars. Statistical analyses were performed using GraphPad Prism 9.5.1. Initially, a one-way analysis of variance (ANOVA) was applied to compare the different treatment groups. Post-hoc analysis was then conducted using Fisher's LSD test to identify significant differences among the groups. A P value less than 0.05 was considered statistically significant.

Before commencing the treatment, cells underwent washing with phosphate-buffered saline (PBS) without Ca²⁺ and Mg²⁺. Following 24 hours of treatment with the precursors, cells were harvested via an initial PBS wash, followed by cell scraping. The cell suspension was subsequently dried overnight using the SPD121P SpeedVac Concentrator. Upon drying, the samples were stored at −80° C. until lipid extraction was performed.

### Results

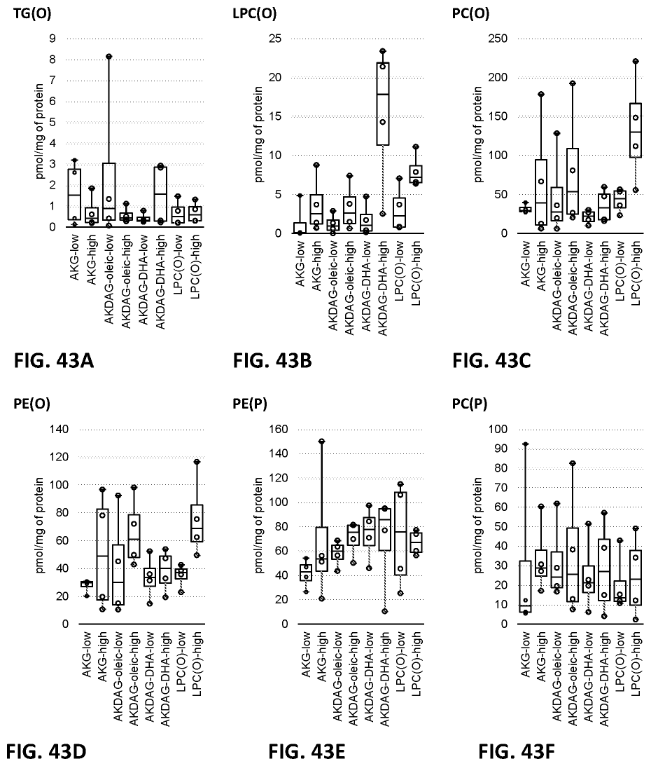

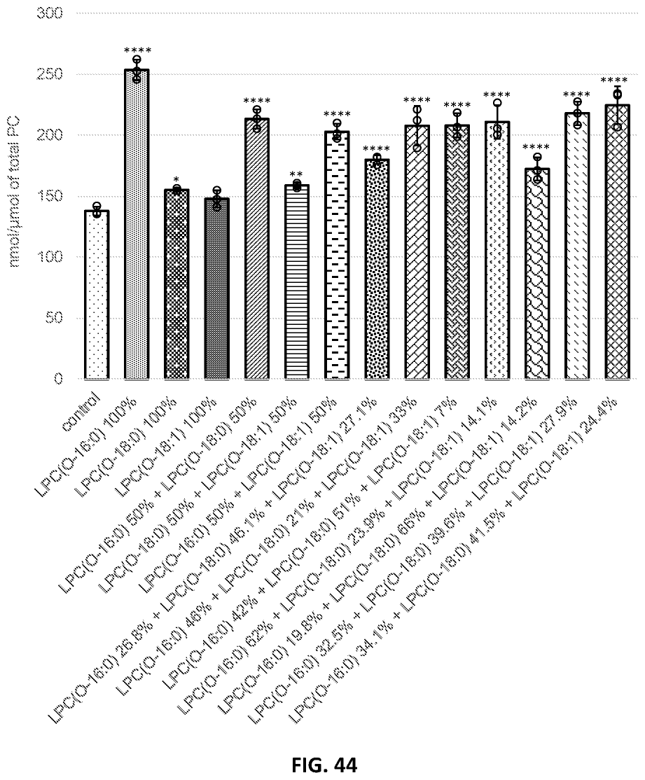

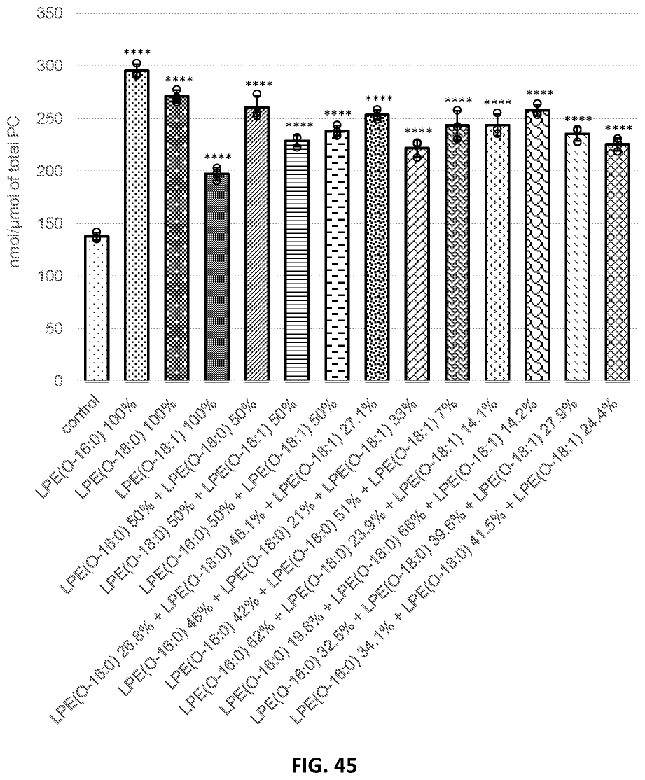

The supplementation of both LPC(O) and LPE(O) resulted in significant increases in the total cellular PE(P) level, albeit to different extents (7-114% relative to control group) (FIG. 44 and FIG. 45). Notably, LPC(O-16:0) 100% exhibited greater efficacy compared to LPC(O-18:0) 100% and LPC(O-18:1) 100% in elevating the endogenous PE(P) level (FIG. 44). Conversely, both LPE(O-16:0) 100% and LPE(O-18:0) 100% showed equal effectiveness in enhancing the PE(P) level, while LPE(O-18:1) 100% was comparatively less effective (FIG. 45). Although different combinations of O-16:0, O-18:0, and O-18:1 species significantly increased the PE(P) level, none of the combinations matched the efficacy of the 100% O-16:0 species in augmenting the PE(P) level (FIG. 43-44).

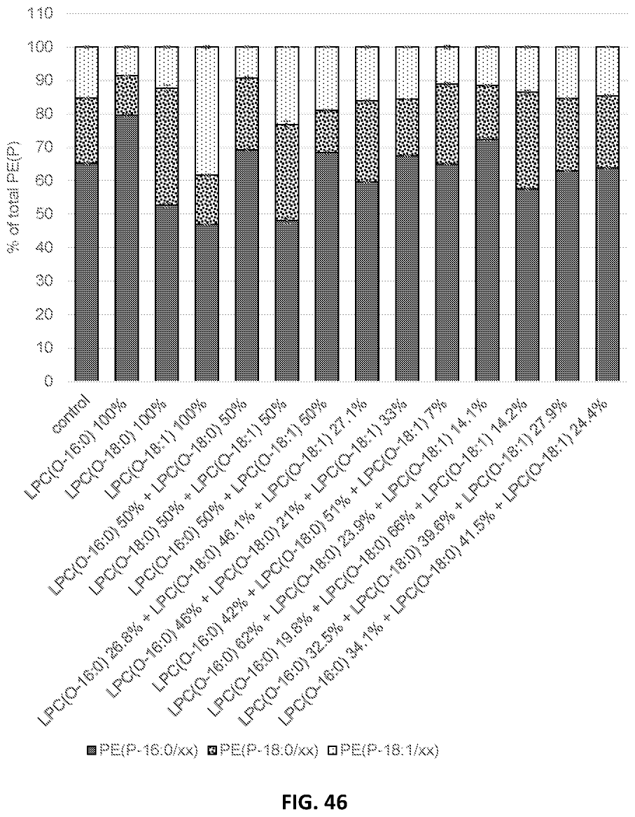

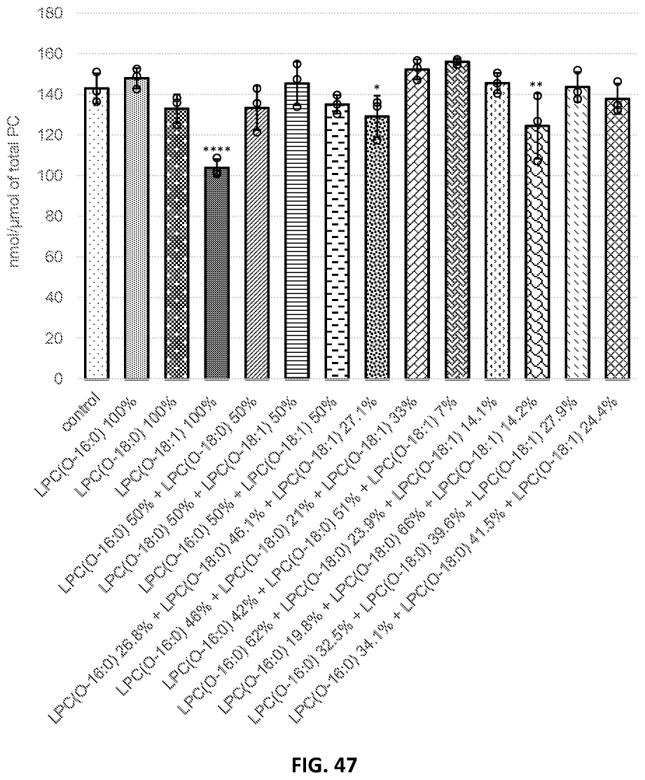

Different combinations of O-16:0, O-18:0, and O-18:1 species also significantly impacted the plasmalogen composition in the cells (FIG. 46 and FIG. 47). Within the LPC(O) treatments, both single and dual species supplementation resulted in alterations in the relative proportions of 16:0, 18:0, and 18:1 PE(P) in cells (FIG. 45). However, specific mixtures, particularly LPC(O-16:0) 34.1%+LPC(O-18:0) 41.5%+LPC(O-18:1) 24.4%, were able to maintain these proportions while increasing the total PE(P) level (FIG. 46). Among the LPE(O) treatments, only one combination—LPE(O-16:0) 62%, LPE(O-18:0) 23.9%, and LPE(O-18:1) 14.1%—successfully preserved the relative proportions of 16:0, 18:0, and 18:1 PE(P) while augmenting the total PE(P) level (FIG. 46). However, all other combinations increased the total PE(P) level while altering the relative proportions of these species.

Without wishing to be bound by theory, it is believed that these results establish the efficacy of novel plasmalogen precursors LPC(O) and LPE(O) in significantly elevating cellular PE(P) levels within RAW 264.7 macrophage cells. Nonetheless, the degree of this increase exhibited significant variation across different SN1 compositions for both LPC(O) and LPE(O) compounds. Notably, O-16:0 at 100% emerged as the most potent in increasing the endogenous PE(P) level compared to the O-18:0 and O-18:1 species. This disparity emphasizes the differential impact of precursor species with different SN1 compositions on cellular plasmalogen levels, potentially attributed to variations in their metabolic processing or their distinct effects on de novo plasmalogen synthesis pathways.

Without being bound by theory, the prevalence of the 16:0 species as the most abundant PE(P) species within these cells suggests a potential preference for accumulating more 16:0 species following supplementation. Notably, alterations in particular PE(P) species are known to inversely affect other species. Consequently, an increase in 18:0 or 18:1 PE(P) species with O-18:0 or O-18:1 precursor supplementation, respectively might trigger stronger feedback inhibition on the de novo synthesis of 16:0 PE(P) species, resulting in a substantially lower overall increase in the total cellular PE(P) level.

The findings illustrate the significant impact of precursor compounds with different SN1 compositions on plasmalogen composition within cells. Specific mixtures within LPC(O) and LPE(O) treatments were able to maintain relative proportions of 16:0, 18:0, and 18:1 PE(P) species while increasing total PE(P) levels, indicating a potential avenue for modulating cellular plasmalogen composition without disrupting the balance of individual species.

Supplementary Table 1: Acquisition details for dynamic multiple reaction
monitoring transitions used in this study.

| | Precursor | Product | Retention Time | Collision |
|---|---|---|---|---|
| Lipid Species | lon | lon | (min) | Energy |
| LPC 14:0 | 468.3 | 184.1 | 1.90 | 21 |
| LPC 15:0 | 482.3 | 184.1 | 2.24 | 21 |
| LPC 16:0 | 496.3 | 184.1 | 2.57 | 21 |
| LPC 16:1 | 494.3 | 184.1 | 2.07 | 21 |
| LPC 17:0 | 510.4 | 184.1 | 2.87 | 21 |
| LPC 17:1 | 508.4 | 184.1 | 2.44 | 21 |
| LPC 18:0 | 524.4 | 184.1 | 3.22 | 21 |
| LPC 18:1 | 522.4 | 184.1 | 2.71 | 21 |
| LPC 18:1 d7 (ISTD) | 529.4 | 184.1 | 2.70 | 21 |
| LPC 18:2 | 520.3 | 184.1 | 2.27 | 21 |
| LPC 18:3 | 518.3 | 184.1 | 1.93 | 21 |
| LPC 18:3(104) | 518.3 | 104.1 | 1.97 | 21 |
| LPC 19:0 | 538.4 | 184.1 | 3.45 | 21 |
| LPC 19:1 | 536.4 | 184.1 | 3.04 | 21 |
| LPC 20:0 | 552.4 | 184.1 | 3.72 | 21 |
| LPC 20:1 | 550.4 | 184.1 | 3.31 | 21 |
| LPC 20:2 | 548.4 | 184.1 | 2.88 | 21 |
| LPC 20:3 | 546.4 | 184.1 | 2.50 | 21 |
| LPC 20:3(104) | 546.4 | 104.1 | 2.55 | 21 |
| LPC 20:4 | 544.3 | 184.1 | 2.24 | 21 |
| LPC 20:4_OH | 560.3 | 184.1 | 1.70 | 21 |
| LPC 20:5 | 542.3 | 184.1 | 1.85 | 21 |
| LPC 22:0 | 580.4 | 184.1 | 4.27 | 21 |
| LPC 22:1 | 578.4 | 184.1 | 3.77 | 21 |
| LPC 22:4 | 572.4 | 184.1 | 2.74 | 21 |
| LPC 22:5(104) | 570.4 | 104.1 | 2.41 | 21 |
| LPC 22:5(a\b\c) | 570.4 | 184.1 | 2.41 | 21 |
| LPC 22:6 | 568.3 | 184.1 | 2.17 | 21 |
| LPC 22:6_OH | 584.3 | 184.1 | 1.61 | 21 |
| LPC 24:0 | 608.5 | 184.1 | 4.97 | 21 |
| LPC 26:0 | 636.5 | 184.1 | 5.86 | 21 |
| LPC(O-16:0) | 482.4 | 104.1 | 2.92 | 23 |
| LPC(O-18:0) | 510.4 | 104.1 | 3.52 | 23 |
| LPC(O-18:1) | 508.4 | 104.1 | 3.03 | 23 |
| LPC(O-20:0) | 538.4 | 104.1 | 4.03 | 23 |
| LPC(O-20:1) | 536.4 | 104.1 | 3.59 | 23 |
| LPC(O-22:0) | 566.5 | 104.1 | 4.69 | 23 |
| LPC(O-22:1) | 564.4 | 104.1 | 4.08 | 23 |
| LPC(O-24:0) | 594.5 | 104.1 | 5.50 | 23 |
| LPC(O-24:1) | 592.5 | 104.1 | 4.74 | 23 |
| LPC(O-24:2) | 590.5 | 104.1 | 4.21 | 23 |
| LPC(P-16:0) | 480.3 | 104.1 | 2.89 | 25 |
| LPC(P-17:0) (a\b) | 494.3 | 104.1 | 3.16 | 25 |
| LPC(P-18:0) | 508.3 | 104.1 | 3.50 | 25 |
| LPC(P-18:1) | 506.3 | 104.1 | 3.02 | 25 |
| LPC(P-20:0) | 536.3 | 104.1 | 4.00 | 25 |
| LPE 16:0 | 454.3 | 313.3 | 2.68 | 17 |
| LPE 18:0 | 482.3 | 341.3 | 3.32 | 17 |
| LPE 18:1 | 480.3 | 339.3 | 2.83 | 17 |
| LPE 18:1 d7 (ISTD) | 487.3 | 346.3 | 2.82 | 17 |
| LPE 18:2 | 478.3 | 337.3 | 2.38 | 17 |
| LPE 20:4 | 502.3 | 361.3 | 2.35 | 17 |
| LPE 22:6 | 526.3 | 385.3 | 2.27 | 17 |
| LPE(O-16:0) | 440.3 | 299.3 | 2.99 | 17 |
| LPE(O-18:0) | 468.3 | 327.3 | 3.57 | 17 |
| LPE(O-18:1) | 466.3 | 325.3 | 3.10 | 17 |
| LPE(P-16:0) | 438.3 | 266.4 | 3.04 | 19 |
| LPE(P-18:0) | 466.3 | 294.4 | 3.61 | 19 |
| LPE(P-18:1) | 464.3 | 292.4 | 3.17 | 19 |
| LPE(P-20:0) | 494.3 | 322.4 | 4.16 | 19 |
| PC 28:0 | 678.5 | 184.1 | 5.14 | 25 |
| PC 30:0 | 706.5 | 184.1 | 5.95 | 25 |
| PC 31:0 (a\b) \ PC(O-32:0) | 720.6 | 184.1 | 7.07 | 25 |
| PC 31:1 \ PC(O-32:1) \ PC(P-32:0) | 718.5 | 184.1 | 6.44 | 25 |
| PC 32:0 | 734.6 | 184.1 | 6.93 | 25 |
| PC 32:1 | 732.6 | 184.1 | 6.15 | 25 |
| PC 32:2 | 730.5 | 184.1 | 5.51 | 25 |
| PC 33:0(a\b) | 748.6 | 184.1 | 7.39 | 25 |
| PC 33:1 \ PC(O-34:1) \ PC(P-34:0) | 746.6 | 184.1 | 7.51 | 25 |
| PC 33:2 \ PC(O-34:2) \ PC(P-34:1) | 744.6 | 184.1 | 6.58 | 25 |
| PC 34:0 | 762.6 | 184.1 | 8.03 | 25 |
| PC 34:1 | 760.6 | 184.1 | 7.13 | 25 |
| PC 34:2 | 758.6 | 184.1 | 6.42 | 25 |
| PC 34:2_OH | 774.6 | 184.1 | 4.73 | 25 |
| PC 34:3(a\b\c) \ PC(P-35:2)(a\b) | 756.6 | 184.1 | 6.51 | 25 |
| PC 34:4 | 754.5 | 184.1 | 5.45 | 25 |
| PC 34:5 \ PC(P-35:4)(a\b) | 752.6 | 184.1 | 5.51 | 25 |
| PC 35:1(a\b) \ PC(O-36:1) | 774.6 | 184.1 | 8.14 | 25 |
| PC 35:2(a\b) \ PC(O-36:2)(a\b) | 772.6 | 184.1 | 7.58 | 25 |
| PC 35:3(a\b) \ PC(O-36:3)(a\b) \ PC(P-36:2)(a\b) | 770.6 | 184.1 | 6.93 | 25 |
| PC 35:4 \ PC(O-36:4) \ PC(P-36:3) | 768.5 | 184.1 | 6.45 | 25 |
| PC 35:5 \ PC(O-36:5) \ PC(P-36:4) | 766.5 | 184.1 | 6.02 | 25 |
| PC 36:1 | 788.6 | 184.1 | 8.26 | 25 |
| PC 36:2(a\b) | 786.6 | 184.1 | 7.37 | 25 |
| PC 36:3(a\b\c) | 784.6 | 184.1 | 6.70 | 25 |
| PC 36:4(a\b) | 782.6 | 184.1 | 6.12 | 25 |
| PC 36:4(a\b)_OH | 798.6 | 184.1 | 5.33 | 25 |
| PC 36:5(a\b) \ PC(P-37:4)(a\b) | 780.5 | 184.1 | 6.35 | 25 |
| PC 36:6 | 778.5 | 184.1 | 5.22 | 25 |
| PC 37:4(a\b) \ PC(O-38:4) | 796.6 | 184.1 | 7.21 | 25 |
| PC 37:6 \ PC(O-38:6) \ PC(P-38:5)(a\b) | 792.6 | 184.1 | 6.29 | 25 |
| PC 38:2 | 814.6 | 184.1 | 8.47 | 25 |
| PC 38:3 | 812.6 | 184.1 | 7.80 | 25 |
| PC 38:4(a\b\c) | 810.6 | 184.1 | 7.13 | 25 |
| PC 38:5(a\b) | 808.6 | 184.1 | 6.61 | 25 |
| PC 38:6(a\b) | 806.6 | 184.1 | 6.02 | 25 |
| PC 38:7(a\b\c) | 804.6 | 184.1 | 5.49 | 25 |
| PC 39:5(a\b) \ PC(O-40:5) \ PC(P-40:4) \ PC 38:6(a\b)_OH | 822.6 | 184.1 | 7.09 | 25 |
| PC 39:6(a\b) \ PC(O-40:6) \ PC(P-40:5)(a\b) | 820.6 | 184.1 | 7.31 | 25 |
| PC 40:4(a\b) | 838.6 | 184.1 | 8.42 | 25 |
| PC 40:5(a\b) | 836.6 | 184.1 | 7.71 | 25 |
| PC 40:6 | 834.6 | 184.1 | 7.17 | 25 |
| PC 40:7(a\b\c) | 832.6 | 184.1 | 6.30 | 25 |
| PC 40:8 | 830.6 | 184.1 | 5.64 | 25 |
| PC 42:10 | 854.5 | 184.1 | 5.54 | 25 |
| PC 42:2 | 870.5 | 184.1 | 10.14 | 25 |
| PC 42:3 | 868.5 | 184.1 | 9.94 | 25 |
| PC 42:4 | 866.5 | 184.1 | 9.29 | 25 |
| PC 42:5 (a/b) | 864.5 | 184.1 | 8.51 | 25 |
| PC 42:6 (a/b) | 862.5 | 184.1 | 7.57 | 25 |
| PC 42:7 | 860.5 | 184.1 | 7.27 | 25 |
| PC 42:8 | 858.5 | 184.1 | 6.42 | 25 |
| PC 42:9 | 856.5 | 184.1 | 5.98 | 25 |
| PC 44:12 | 878.6 | 184.1 | 5.37 | 25 |
| PC 44:4 | 894.5 | 184.1 | 10.07 | 25 |
| PC 44:5 | 892.5 | 184.1 | 9.80 | 25 |
| PC(15:0_18:1) d7 (ISTD) | 753.6 | 184.1 | 6.59 | 25 |
| PC(O-32:2) \ PC(P-32:1) | 716.6 | 184.1 | 6.44 | 25 |
| PC(O-34:4) \ PC(P-34:3) | 740.6 | 184.1 | 5.97 | 25 |
| PC(O-36:0) | 776.6 | 184.1 | 9.95 | 25 |
| PC(O-38:5) \ PC(P-38:4) | 794.6 | 184.1 | 7.39 | 25 |
| PC(O-40:7) \ PC(P-40:6) | 818.6 | 184.1 | 7.09 | 25 |
| PC(O-42:4) (a/b) | 852.5 | 184.1 | 9.94 | 25 |
| PC(O-42:5) (a/b/c) | 850.5 | 184.1 | 9.55 | 25 |
| PC(O-42:6) \ PC(P-42:5) | 848.5 | 184.1 | 8.73 | 25 |
| PC(O-42:7) | 846.5 | 184.1 | 7.98 | 25 |
| PC(O-42:8) | 844.5 | 184.1 | 7.49 | 25 |
| PC(O-44:6) \ PC(P-44:5) | 876.5 | 184.1 | 9.90 | 25 |
| PC(O-44:7) | 874.5 | 184.1 | 9.15 | 25 |
| PC(O-46:7) (a/b) | 902.5 | 184.1 | 9.92 | 25 |
| PC(O-46:8) | 900.5 | 184.1 | 9.37 | 25 |
| PC(P-18:0/18:1) d9 (ISTD) | 781.6 | 184.1 | 8.86 | 25 |
| PC(P-30:0) | 690.4 | 184.1 | 6.48 | 25 |
| PC(P-34:2) | 742.5 | 184.1 | 6.94 | 25 |
| PC(P-36:5) | 764.6 | 184.1 | 6.22 | 25 |
| PC(P-38:6) | 790.6 | 184.1 | 6.63 | 25 |
| PC(P-40:7) | 816.6 | 184.1 | 6.87 | 25 |
| PC(P-46:8) | 898.5 | 184.1 | 9.19 | 25 |
| PE 32:0 | 692.5 | 551.5 | 7.28 | 19 |
| PE 32:1 | 690.5 | 549.5 | 6.47 | 19 |
| PE 34:1 | 718.5 | 577.5 | 7.49 | 19 |
| PE 34:2 | 716.5 | 575.5 | 6.73 | 19 |
| PE 34:3(a\b\c) | 714.5 | 573.5 | 6.10 | 19 |
| PE 35:1(a\b) | 732.6 | 591.5 | 7.96 | 19 |
| PE 35:2(a\b) | 730.5 | 589.5 | 7.17 | 19 |
| PE 36:0 | 748.6 | 607.6 | 9.62 | 19 |
| PE 36:1 | 746.6 | 605.6 | 8.66 | 19 |
| PE 36:2(a\b) | 744.6 | 603.5 | 7.74 | 19 |
| PE 36:3(a\b) | 742.5 | 601.5 | 6.97 | 19 |
| PE 36:4 | 740.5 | 599.5 | 6.67 | 19 |
| PE 36:5(a\b) | 738.5 | 597.5 | 5.96 | 19 |
| PE 37:4 (a\b) | 754.6 | 613.5 | 7.10 | 19 |
| PE 38:3(a\b) | 770.6 | 629.6 | 8.39 | 19 |
| PE 38:4 | 768.6 | 627.5 | 7.76 | 19 |
| PE 38:5(a\b) | 766.5 | 625.5 | 6.95 | 19 |
| PE 38:6 | 764.5 | 623.5 | 6.47 | 19 |
| PE 39:6(a\b) \ PE(O-40:6) | 778.5 | 637.5 | 7.50 | 19 |
| PE 40:4 | 796.6 | 655.6 | 8.80 | 19 |
| PE 40:5(a\b) | 794.6 | 653.6 | 8.09 | 19 |
| PE 40:6 | 792.6 | 651.5 | 7.52 | 19 |
| PE 40:7 | 790.5 | 649.5 | 6.65 | 19 |
| PE(15:0_18:1) d7 (ISTD) | 711.6 | 570.5 | 6.92 | 19 |
| PE(O-34:1) | 704.6 | 563.5 | 8.26 | 19 |
| PE(O-34:2) | 702.5 | 561.5 | 7.47 | 19 |
| PE(O-36:3)(a\b) | 728.6 | 587.5 | 7.73 | 19 |
| PE(O-36:4) | 726.5 | 585.5 | 7.40 | 19 |
| PE(O-36:5) | 724.5 | 583.5 | 6.76 | 19 |
| PE(O-38:4)(a\b) | 754.6 | 613.6 | 8.39 | 19 |
| PE(O-38:5)(a\b) | 752.6 | 611.5 | 7.72 | 19 |
| PE(O-38:6) | 750.6 | 609.5 | 7.16 | 19 |
| PE(O-40:5) | 780.6 | 639.6 | 8.65 | 19 |
| PE(O-40:7) | 776.6 | 635.5 | 7.30 | 19 |
| PE(P-15:0/20:4)(a\b) | 710.5 | 361.3 | 6.63 | 19 |
| PE(P-15:0/22:6)(a\b) | 734.5 | 385.3 | 6.42 | 19 |
| PE(P-16:0/18:1) | 702.5 | 339.3 | 8.15 | 19 |
| PE(P-16:0/18:2) | 700.5 | 337.3 | 7.34 | 19 |
| PE(P-16:0/18:3) | 698.5 | 335.3 | 6.71 | 19 |
| PE(P-16:0/20:3) | 726.5 | 363.3 | 7.73 | 19 |
| PE(P-16:0/20:4) | 724.5 | 361.3 | 7.23 | 19 |
| PE(P-16:0/20:5) | 722.5 | 359.3 | 6.57 | 19 |
| PE(P-16:0/22:4) | 752.6 | 389.3 | 8.05 | 19 |
| PE(P-16:0/22:5)(a\b) | 750.5 | 387.3 | 7.52 | 19 |
| PE(P-16:0/22:6) | 748.5 | 385.3 | 7.00 | 19 |
| PE(P-17:0/20:4)(a\b) | 738.6 | 361.3 | 7.70 | 19 |
| PE(P-17:0/22:6)(a\b) | 762.6 | 385.3 | 7.49 | 19 |
| PE(P-18:0/18:1) | 730.6 | 339.3 | 9.37 | 19 |
| PE(P-18:0/18:1) d9 (ISTD) | 739.5 | 348.3 | 9.32 | 19 |
| PE(P-18:0/18:2) | 728.6 | 337.3 | 8.50 | 19 |
| PE(P-18:0/18:3) | 726.5 | 335.3 | 7.85 | 19 |
| PE(P-18:0/20:3) | 754.5 | 363.3 | 8.94 | 19 |
| PE(P-18:0/20:4) | 752.6 | 361.3 | 8.39 | 19 |
| PE(P-18:0/20:5) | 750.5 | 359.3 | 7.67 | 19 |
| PE(P-18:0/22:4) | 780.6 | 389.3 | 9.27 | 19 |
| PE(P-18:0/22:5)(a\b) | 778.5 | 387.3 | 8.71 | 19 |
| PE(P-18:0/22:6) | 776.6 | 385.3 | 8.14 | 19 |
| PE(P-18:1/18:1) | 728.6 | 339.3 | 8.35 | 19 |
| PE(P-18:1/18:2) | 726.5 | 337.3 | 7.50 | 19 |
| PE(P-18:1/18:3) | 724.5 | 335.3 | 6.85 | 19 |
| PE(P-18:1/20:3) | 752.5 | 363.3 | 7.89 | 19 |
| PE(P-18:1/20:4) | 750.5 | 361.3 | 7.45 | 19 |
| PE(P-18:1/20:5) | 748.5 | 359.3 | 6.77 | 19 |
| PE(P-18:1/22:4) | 778.5 | 389.3 | 8.27 | 19 |
| PE(P-18:1/22:5)(a\b) | 776.6 | 387.3 | 7.67 | 19 |
| PE(P-18:1/22:6)(a\b) | 774.5 | 385.3 | 7.24 | 19 |
| PE(P-19:0/20:4)(a\b) | 766.6 | 361.3 | 8.92 | 19 |
| PE(P-20:0/18:1) | 758.6 | 339.3 | 10.03 | 19 |
| PE(P-20:0/18:2) | 756.6 | 337.3 | 9.73 | 19 |
| PE(P-20:0/20:4) | 780.6 | 361.3 | 9.63 | 19 |
| PE(P-20:0/22:6) | 804.6 | 385.3 | 9.38 | 19 |
| PE(P-20:1/20:4) | 778.5 | 361.3 | 8.55 | 19 |
| PE(P-20:1/22:6) | 802.6 | 385.3 | 8.25 | 19 |
| TG(48:0) [NL-16:0] | 824.8 | 551.5 | 11.26 | 25 |
| TG(48:0) [NL-18:0] | 824.8 | 523.5 | 11.26 | 25 |
| TG(48:0) [SIM] | 824.8 | 824.8 | 11.25 | 0 |
| TG(48:1) [NL-16:1] | 822.8 | 551.5 | 11.09 | 25 |
| TG(48:1) [NL-18:1] | 822.8 | 523.5 | 11.09 | 25 |
| TG(48:1) [NL-18:1] d7 (ISTD) | 829.8 | 523.5 | 11.07 | 25 |
| TG(48:1) [SIM] | 822.8 | 822.8 | 11.08 | 0 |
| TG(48:2) [NL-14:0] | 820.8 | 575.5 | 10.94 | 25 |
| TG(48:2) [NL-14:1] | 820.8 | 577.5 | 10.93 | 25 |
| TG(48:2) [NL-16:1] | 820.8 | 549.5 | 10.92 | 25 |
| TG(48:2) [NL-18:2] | 820.8 | 523.5 | 10.94 | 25 |
| TG(48:2) [SIM] | 820.8 | 820.8 | 10.94 | 0 |
| TG(48:3) [NL-14:0] | 818.8 | 573.5 | 10.80 | 25 |
| TG(48:3) [NL-16:1] | 818.8 | 547.5 | 10.78 | 25 |
| TG(48:3) [NL-18:3] | 818.8 | 523.5 | 10.83 | 25 |
| TG(48:3) [SIM] | 818.8 | 818.8 | 10.79 | 0 |
| TG(49:1) [NL-16:1] | 836.8 | 565.5 | 11.18 | 25 |
| TG(49:1) [NL-17:1] \ TG(O-50:1) [NL-17:1] | 836.8 | 551.5 | 11.44 | 25 |
| TG(49:1) [SIM] | 836.8 | 836.8 | 11.18 | 0 |
| TG(50:0) [NL-18:0] | 852.8 | 551.5 | 11.47 | 25 |
| TG(50:0) [SIM] | 852.8 | 852.8 | 11.47 | 0 |
| TG(50:1) [NL-14:0] | 850.8 | 605.5 | 11.26 | 25 |
| TG(50:1) [NL-16:0] | 850.8 | 577.5 | 11.26 | 25 |
| TG(50:1) [NL-18:1] | 850.8 | 551.5 | 11.27 | 25 |
| TG(50:1) [SIM] | 850.8 | 850.8 | 11.26 | 0 |
| TG(50:2) [NL-14:0] | 848.8 | 603.5 | 11.10 | 25 |
| TG(50:2) [NL-16:1] | 848.8 | 577.5 | 11.10 | 25 |
| TG(50:2) [NL-18:1] | 848.8 | 549.5 | 11.10 | 25 |
| TG(50:2) [NL-18:2] | 848.8 | 551.5 | 11.12 | 25 |
| TG(50:2) [SIM] | 848.8 | 848.8 | 11.10 | 0 |
| TG(50:3) [NL-14:0] | 846.8 | 601.5 | 10.95 | 25 |
| TG(50:3) [NL-14:1] | 846.8 | 603.5 | 10.96 | 25 |
| TG(50:3) [NL-16:1] | 846.8 | 575.5 | 10.95 | 25 |
| TG(50:3) [NL-18:2] | 846.8 | 549.5 | 10.96 | 25 |
| TG(50:3) [NL-18:3] | 846.8 | 551.5 | 11.00 | 25 |
| TG(50:3) [SIM] | 846.8 | 846.8 | 10.96 | 0 |
| TG(50:4) [NL-14:0] | 844.8 | 599.5 | 10.82 | 25 |
| TG(50:4) [NL-18:3] | 844.8 | 549.5 | 10.84 | 25 |
| TG(50:4) [NL-20:4] | 844.8 | 523.5 | 10.91 | 25 |
| TG(50:4) [SIM] | 844.8 | 844.8 | 10.83 | 0 |
| TG(51:0) [NL-16:0] | 866.7 | 593.4 | 11.56 | 25 |
| TG(51:0) [SIM] | 866.7 | 866.7 | 11.56 | 0 |
| TG(51:1) [NL-17:0] | 864.8 | 577.5 | 11.36 | 25 |
| TG(51:1) [SIM] | 864.8 | 864.8 | 11.36 | 0 |
| TG(51:2) [NL-15:0] | 862.8 | 603.5 | 11.20 | 25 |
| TG(51:2) [NL-17:0] | 862.8 | 575.5 | 11.19 | 25 |
| TG(51:2) [NL-17:1] \ TG(O-52:2) [NL-17:1] | 862.8 | 577.5 | 11.42 | 25 |
| TG(51:2) [SIM] | 862.8 | 862.8 | 11.19 | 0 |
| TG(52:1) [NL-18:0] | 878.8 | 577.5 | 11.48 | 25 |
| TG(52:1) [NL-18:1] | 878.8 | 579.5 | 11.48 | 25 |
| TG(52:1) [SIM] | 878.8 | 878.8 | 11.48 | 0 |
| TG(52:2) [NL-16:0] | 876.8 | 603.5 | 11.29 | 25 |
| TG(52:2) [NL-18:2] | 876.8 | 579.5 | 11.31 | 25 |
| TG(52:2) [SIM] | 876.8 | 876.8 | 11.31 | 0 |
| TG(52:3) [NL-16:1] | 874.8 | 603.5 | 11.12 | 25 |
| TG(52:3) [NL-18:2] | 874.8 | 577.5 | 11.14 | 25 |
| TG(52:3) [SIM] | 874.8 | 874.8 | 11.13 | 0 |
| TG(52:4) [NL-16:1] | 872.8 | 601.5 | 10.98 | 25 |
| TG(52:4) [NL-18:2] | 872.8 | 575.5 | 10.98 | 25 |
| TG(52:4) [NL-18:3] | 872.8 | 577.5 | 11.01 | 25 |
| TG(52:4) [SIM] | 872.8 | 872.8 | 10.99 | 0 |
| TG(52:5) [NL-18:3] | 870.8 | 575.5 | 10.87 | 25 |
| TG(52:5) [NL-20:4] | 870.8 | 549.5 | 10.92 | 25 |
| TG(52:5) [NL-20:5] | 870.8 | 551.5 | 10.94 | 25 |
| TG(52:5) [SIM] | 870.8 | 870.8 | 10.86 | 0 |
| TG(53:2) [NL-17:1] | 890.8 | 605.5 | 11.47 | 25 |
| TG(53:2) [NL-18:1] \ TG(O-54:2) [NL-18:1] | 890.8 | 591.5 | 11.64 | 25 |
| TG(53:2) [SIM] | 890.8 | 890.8 | 11.37 | 0 |
| TG(54:0) [NL-18:0] | 908.8 | 607.5 | 11.89 | 25 |
| TG(54:0) [SIM] | 908.8 | 908.8 | 11.89 | 0 |
| TG(54:1) [NL-18:1] | 906.8 | 607.5 | 11.69 | 25 |
| TG(54:1) [SIM] | 906.8 | 906.8 | 11.69 | 0 |
| TG(54:2) [NL-18:0] | 904.8 | 603.5 | 11.49 | 25 |
| TG(54:2) [NL-20:1] | 904.8 | 577.5 | 11.49 | 25 |
| TG(54:2) [SIM] | 904.8 | 904.8 | 11.49 | 0 |
| TG(54:3) [NL-18:1] | 902.8 | 603.5 | 11.31 | 25 |
| TG(54:3) [NL-18:2] | 902.8 | 605.5 | 11.33 | 25 |
| TG(54:3) [SIM] | 902.8 | 902.8 | 11.31 | 0 |
| TG(54:4) [NL-18:2] | 900.8 | 603.5 | 11.16 | 25 |
| TG(54:4) [NL-20:3] | 900.8 | 577.5 | 11.22 | 25 |
| TG(54:4) [SIM] | 900.8 | 900.8 | 11.15 | 0 |
| TG(54:5) [NL-18:3] | 898.8 | 603.5 | 11.02 | 25 |
| TG(54:5) [NL-20:4] | 898.8 | 577.5 | 11.08 | 25 |
| TG(54:5) [SIM] | 898.8 | 898.8 | 11.02 | 0 |
| TG(54:6) [NL-18:3] | 896.8 | 601.5 | 10.88 | 25 |
| TG(54:6) [NL-20:4] | 896.8 | 575.5 | 10.94 | 25 |
| TG(54:6) [NL-20:5] | 896.8 | 577.5 | 10.95 | 25 |
| TG(54:6) [NL-22:6] | 896.8 | 551.5 | 11.01 | 25 |
| TG(54:6) [SIM] | 896.8 | 896.8 | 10.88 | 0 |
| TG(54:7) [NL-20:5] | 894.8 | 575.5 | 10.81 | 25 |
| TG(54:7) [NL-22:6] | 894.8 | 549.5 | 10.86 | 25 |
| TG(54:7) [SIM] | 894.8 | 894.8 | 10.83 | 0 |
| TG(56:6) [NL-20:4] | 924.8 | 603.5 | 11.11 | 25 |
| TG(56:6) [NL-22:5] | 924.8 | 577.5 | 11.08 | 25 |
| TG(56:6) [SIM] | 924.8 | 924.8 | 11.09 | 0 |
| TG(56:7) [NL-20:4] | 922.8 | 601.5 | 10.96 | 25 |
| TG(56:7) [NL-20:5] | 922.8 | 603.5 | 10.97 | 25 |
| TG(56:7) [NL-22:5] | 922.8 | 575.5 | 10.94 | 25 |
| TG(56:7) [NL-22:6] | 922.8 | 577.5 | 11.02 | 25 |
| TG(56:7) [SIM] | 922.8 | 922.8 | 11.02 | 0 |
| TG(56:8) [NL-20:4] | 920.8 | 599.5 | 10.83 | 25 |
| TG(56:8) [NL-20:5] | 920.8 | 601.5 | 10.84 | 25 |
| TG(56:8) [NL-22:6] | 920.8 | 575.5 | 10.88 | 25 |
| TG(56:8) [SIM] | 920.8 | 920.8 | 10.88 | 0 |
| TG(56:9) [NL-22:6] | 918.8 | 573.5 | 10.76 | 25 |
| TG(56:9) [SIM] | 918.8 | 918.8 | 10.74 | 0 |
| TG(58:10) [NL-22:6] | 944.9 | 599.5 | 10.77 | 25 |
| TG(58:10) [SIM] | 944.9 | 944.9 | 10.77 | 0 |
| TG(58:8) [NL-22:6] | 948.8 | 603.5 | 11.03 | 25 |
| TG(58:8) [SIM] | 948.8 | 948.8 | 11.00 | 0 |
| TG(58:9) [NL-22:6] | 946.9 | 601.5 | 10.90 | 25 |
| TG(58:9) [SIM] | 946.9 | 946.9 | 10.90 | 0 |
| TG(O-50:1) [NL-15:0] | 836.8 | 577.5 | 11.61 | 25 |
| TG(O-50:1) [NL-16:0] | 836.8 | 563.5 | 11.64 | 25 |
| TG(O-50:1) [NL-18:1] | 836.8 | 537.5 | 11.62 | 25 |
| TG(O-50:2) [NL-16:1] | 834.8 | 563.5 | 11.50 | 25 |
| TG(O-50:2) [NL-18:1] | 834.8 | 535.5 | 11.47 | 25 |
| TG(O-50:2) [NL-18:2] | 834.8 | 537.5 | 11.49 | 25 |
| TG(O-50:3) [NL-18:2] | 832.8 | 535.5 | 11.25 | 25 |
| TG(O-52:0) [NL-16:0] | 866.8 | 593.5 | 12.05 | 25 |
| TG(O-52:1) [NL-16:0] | 864.8 | 591.5 | 11.85 | 25 |
| TG(O-52:1) [NL-18:1] | 864.8 | 565.5 | 11.85 | 25 |
| TG(O-52:2) [NL-16:0] | 862.8 | 589.5 | 11.62 | 25 |
| TG(O-52:2) [NL-18:1] | 862.8 | 563.5 | 11.62 | 25 |
| TG(O-54:3) [NL-17:1] | 888.8 | 603.5 | 11.78 | 25 |
| TG(O-54:3) [NL-18:1] | 888.8 | 589.5 | 11.63 | 25 |
| TG(O-54:4) [NL-17:1] | 886.8 | 601.5 | 11.58 | 25 |
| TG(O-54:4) [NL-18:2] | 886.8 | 589.5 | 11.49 | 25 |

For all transitions, the polarity was positive, with fragmentor 166 and cell accelerator voltage set to 5.

LPC: lysophosphatidylcholine; LPC(O): lysoalkylphosphatidylcholine; LPC(P): lysoalkenylphosphatidylcholine; LPE: lysophosphatidylethanolamine; LPE(O): lysoalkylphosphatidylethanolamine; LPE(P): lysoalkenylphosphatidylethanolamine; PC: phosphatidylcholine; PC(O): alkylphosphatidylcholine; PC(P): alkenylphosphatidylcholine; PE: phosphatidylethanolamine; PE(O): alkylphosphatidylethanolamine; PE(P): alkenylphosphatidylethanolamine [PE(P)]; TG: triacylglycerol; TG(O): alkyldiacylglycerol, ISTD: internal standard; NL: neutral loss; SIM: single ion monitoring.

### Example 6

This example relates to modulation of plasmalogen level and composition by LPC(O) and LPE(O) supplementation in 3T3-L1 cells using different SN-1 compositions.

The method employed in this study was the same as described in example 5, with the exception of the cell line and culture medium used. For this study, a mouse fibroblast/pre-adipocyte cell line, 3T3-L1, was utilised and cultured in Dulbecco's Modified Eagle Medium (DMEM) (Gibco, USA), supplemented with 1% sodium pyruvate (Gibco, USA) and 10% newborn calf serum (Gibco, New Zealand).

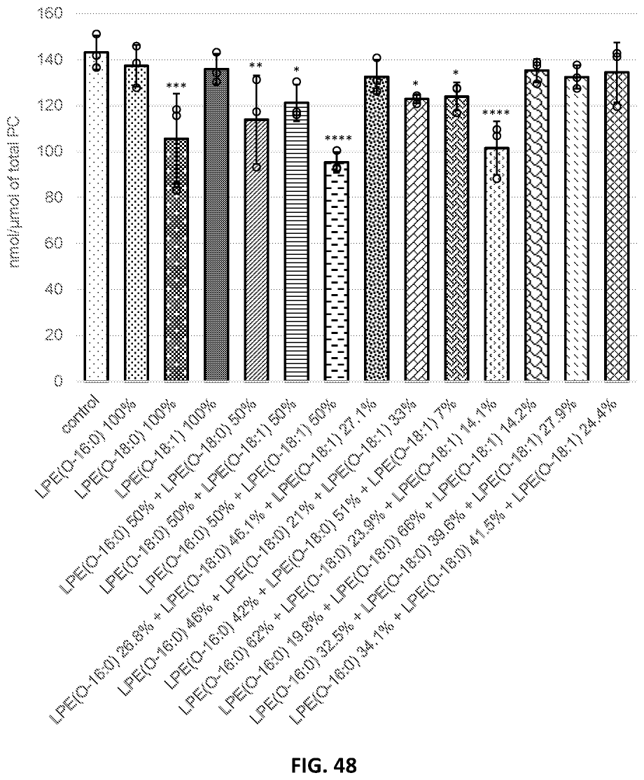

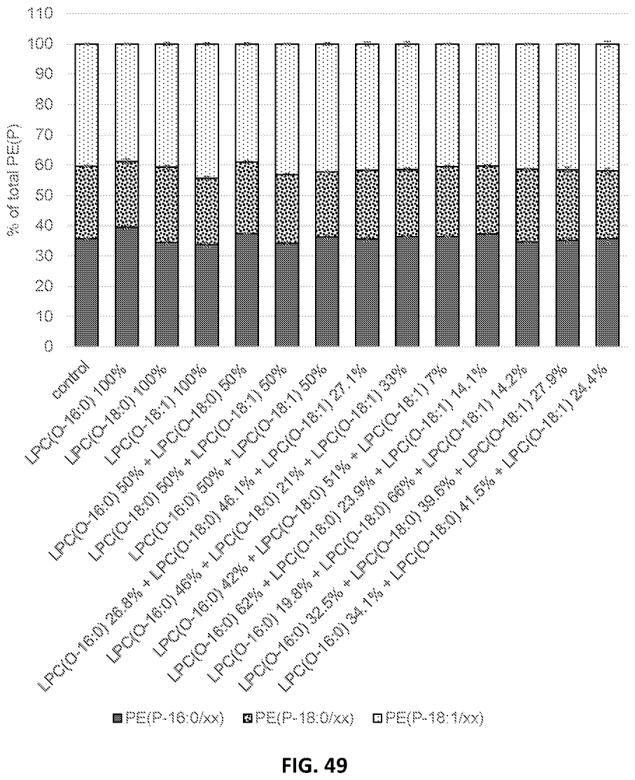

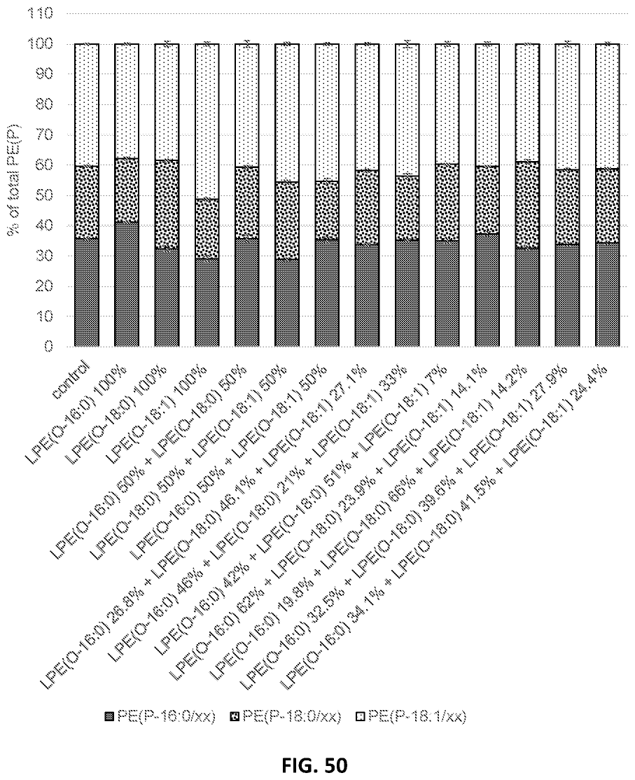

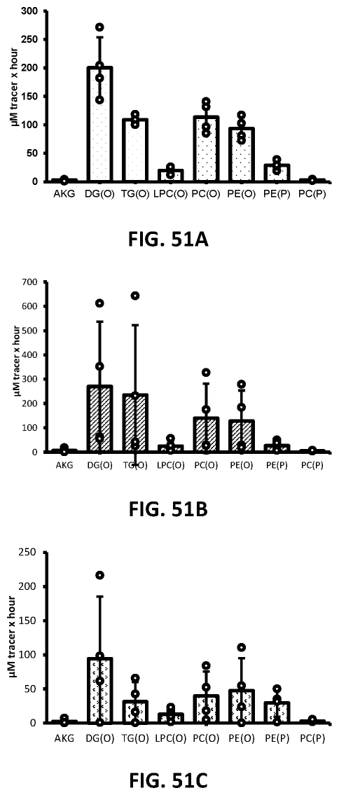

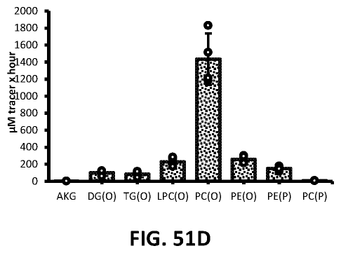

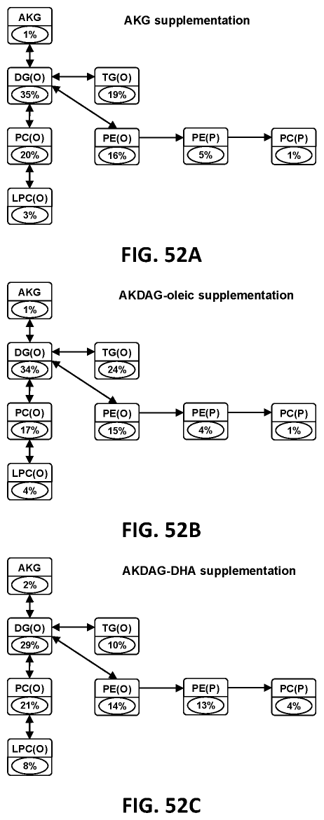

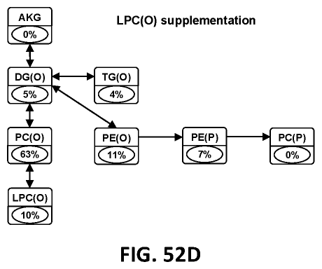

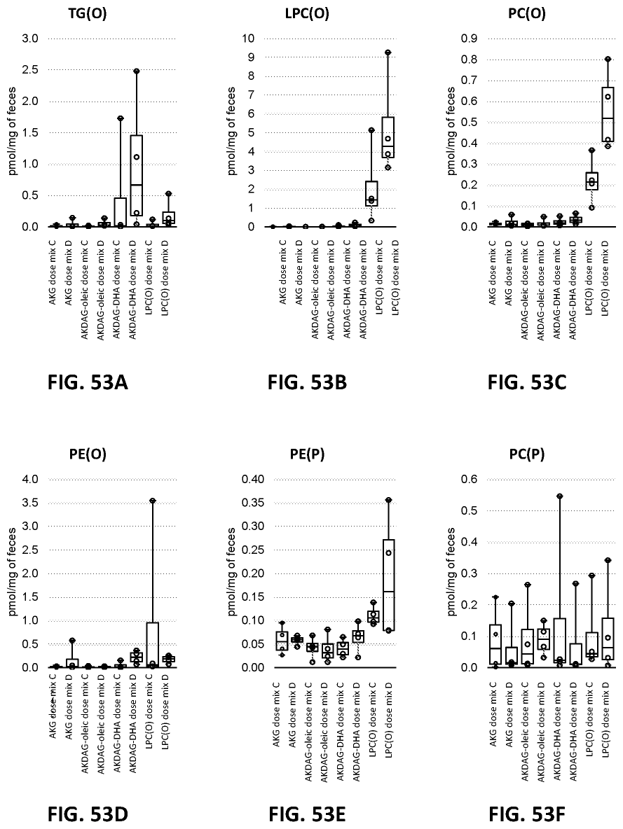

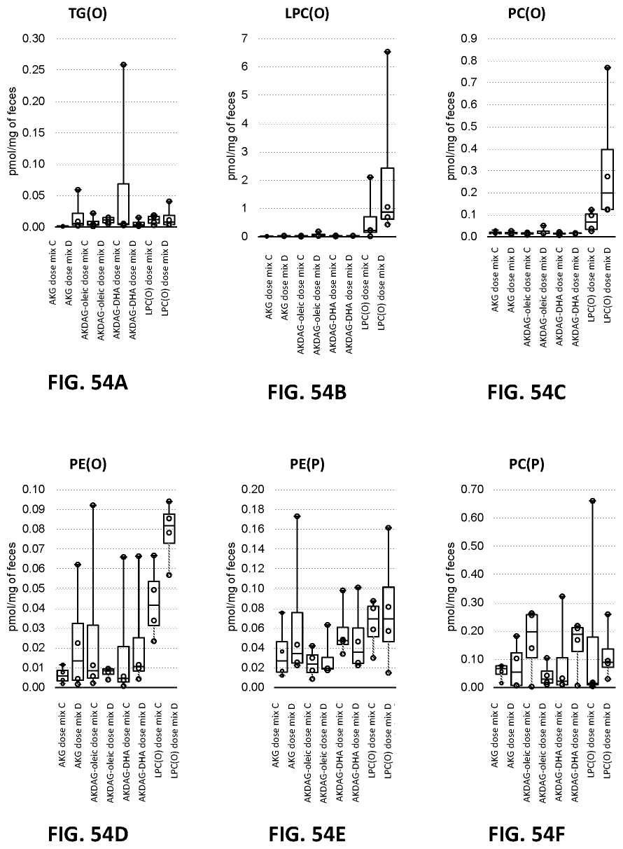

In 3T3-L1 pre-adipocyte cells, none of the plasmalogen precursor treatments resulted in a significant increase in cellular PE(P) levels; rather, some treatments, notably LPC(O-18:1) 100% and LPE(O-18:1) 100% appeared to reduce these levels (FIG. 47-48). Exploring the effects within both LPC(O) and LPE(O) treatments, single species supplementation revealed subtle shifts in the relative proportions of 16:0, 18:0, and 18:1 PE(P), i.e., supplementation of a particular SN1 species increased the proportion of corresponding PE(P) species while decreased the proportions of other PE(P) species (FIG. 49-50).

In 3T3-L1 cells, increases in cellular PE(P) levels following supplementation with LPC(O) or LPE(O) were not observed under the conditions tested. These cells already exhibit high baseline levels of PE(P), potentially accounting for the lack of further increase with precursor supplementation. Nevertheless, the studies revealed a significant influence of precursor compounds with different SN1 compositions on the cellular PE(P) composition. Without wishing to be bound by theory, it is believed that these results emphasize the role of the SN1/alkyl chain composition in plasmalogen precursor supplements for preserving the natural plasmalogen composition in healthy subjects.

### Example 7

The mouse macrophage cell line RAW 264.7 was used for additional studies to determine if any functional/phenotypic outcome could be measured as a result of the increased plasmalogen levels/composition changes that were observed by feeding LPC(O) and LPE(O) ether lipids to the cells.

A subset of the Table 14 compound mixtures with varying SN-1 fatty acids was chosen based upon ratios that appeared to maintain the plasmalogen compound ratios most often observed in healthy human subjects. The compound mixtures tested were:

### Vehicle Control

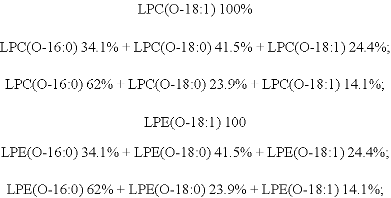

The cells were pretreated with 20 μM of precursor compound mixtures for 24 hours, and then treated with LPS (1 nM) for 6 hours to stimulate an immune response. After LPS treatment, the cells were harvested for gene expression analysis using qPCR. Total RNA was isolated from the cells using TRIzol™ reagent (Invitrogen, Thermo Fisher Scientific, USA). 500 μl of TRIzol was added to each well of the cell culture plate, followed by the use of a scraper to lift the cells from the plate. The resulting cell lysates were stored at −80° C. until subsequent processing. On the day of RNA isolation, the cell lysates were thawed, and 100 μl of chloroform was added to each sample. Each sample tube was then vigorously mixed for 20 seconds and then incubated at room temperature for 5 minutes. Subsequently, the samples were centrifuged at 4° C. at 12000 g for 15 minutes.

The aqueous phase was carefully transferred to fresh tubes. 500 μl of isopropanol were added to each sample and thoroughly mixed before incubating at room temperature for 30 minutes. The samples were then centrifuged at 4° C. at 17000 g for 15 minutes. The supernatant was decanted, leaving the RNA pellet.

To the RNA pellet, 1 ml of 75% molecular-grade ethanol (Sigma-Aldrich, USA) was added, briefly vortexed for 5 seconds, and centrifuged at 4° C. for 5 minutes at 7500 g. The supernatant was then discarded. Subsequently, an additional 1 ml of 75% ice-cold ethanol was added to the pellet, followed by centrifugation at 4° C. for 5 minutes at 7500 g. This washing process was repeated once more, and after the final centrifugation, the supernatant was discarded, leaving the cleaned RNA pellet.

The tubes containing the pellet were dried on a heating block at 55° C. for 10 minutes. Once dried, 20 μl of molecular-grade water (Sigma-Aldrich, USA) was added to dissolve the RNA pellet. The quantity and quality of the isolated RNA were determined using a Nanodrop spectrophotometer (Thermo Fisher Scientific, USA) and the RNA samples were stored at −80° C. until further analysis.

Complementary DNA was synthesised by reverse transcription using a High Capacity cDNA Reverse Transcription Kit (Applied Biosystems, Thermo Fisher Scientific, USA) according to the manufacturer's recommendations.

Quantitative real-time PCR was performed using Taqman™ assays (Il6: Mm00446190_m1, Nfe212: Mm00477784_m1, Tlr4: Mm00445273_m1, Acox1: Mm00443579_m1, Cpt1α: Mm00550438_m1, Sod1: Mm01344233_g1, Pex16: Mm00455021_m1, Hnrnpab: Mm01288699_m1) and TaqMan™ Fast Advanced Master Mix (Applied Biosystems, Thermo Fisher Scientific, USA), and amplified on an Applied Biosystems Quant 7 real time PCR instrument (Life Technologies, Thermo Fisher Scientific, USA) following manufacturer's recommendations. Target gene expression was normalized to a housekeeping gene [heterogeneous nuclear ribonucleoprotein (Hnrnpab)] expression and a reference group (control or control+LPS) using the 2-ΔΔCt method of quantification and expressed as a relative value (Livak K J, Schmittgen T D. Methods. Vol. 25. San Diego, CA: 2001. Analysis of relative gene expression data using real-time quantitative PCR and the 2(−Delta Delta C(T)) Method; pp.

402-408.)

Data are presented as mean±SD (n=3/group). Mean difference between the groups were compared by one-way ANOVA followed by Fishers LSD test. An asterisk (*) indicates P<0.05 vs control and \hat{ } indicates P<0.05 vs control+LPS.

While none of the compounds tested appeared to impact the level of TNFα, as measured by ELISA (data not shown), there was an effect seen at the gene expression level for many indicators of inflammatory/immune response.

LPC(O-18:1) supplementation suppressed the LPS-induced increase in IL6 gene expression as did all the LPE(O) treatments. All of the LPX(O) treatments reduced the NFE2L2 gene expression, although only 4 of the 6 (mixes 4-7 above, including all the LPE(O)s), were statistically significant in this assay.

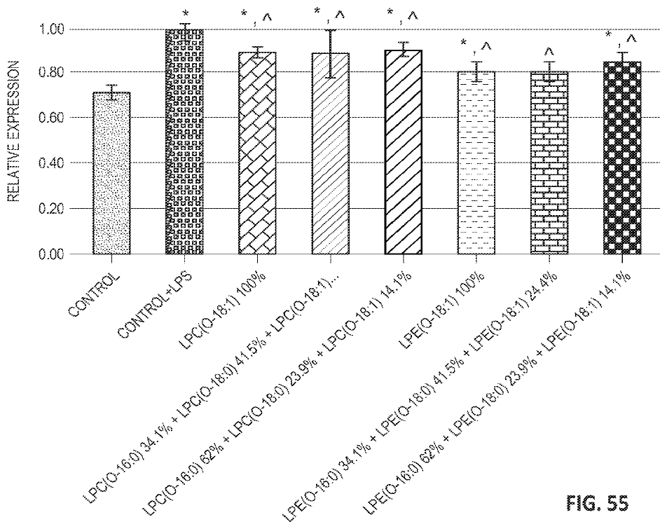

All of the LPX(O) treatments suppressed the LPS-induced increase in TLR4 gene expression (FIG. 55). A range of 11-20% reduction was noted as compared to the amount of induction after LPS treatment, subtracting the control vehicle level.

These data suggest that there may be an anti-inflammatory activity associated with LPX(O) supplementation, particularly LPE(O) in RAW 264.7 cells (Xing, X. et al., IL-6 is an anti-inflammatory cytokine required for controlling local or systemic acute inflammatory responses. J Clin Invest. 1998; 15:311-320; He, F. et al. 2020 NRF2, a transcription factor for stress response and beyond. Int. J. Mol. Sci 2020:21(13), 4777; Saleh et al., The anti-inflammatory properties of phytochemicals and their effects on epigenetic mechanisms involved in TLR4/NF-kB-mediated inflammation. Front. Immunol. 2021:12.)

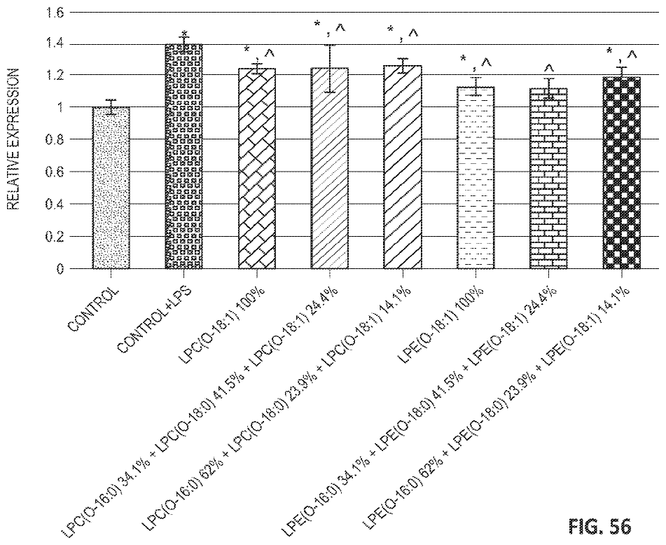

RAW 264.7 macrophage cells were treated with 20 μM of LPC(O) or LPE(O) with different SN1 compositions for 24 hours and further treated with LPS (1 nM) for 6 hours and then harvested for gene expression analysis. Gene expression data are normalised to Hnrnpab expression and presented as mean±SD (n=3/group). Mean difference between the groups were compared by one-way ANOVA followed by Fishers LSD test. Results are depicted in FIG. 56, where *indicates P<0.05 vs control, \hat{ } indicates P<0.05 vs Control+LPS.

### Example 8

### Formulations of LPX(O) Compounds.

Certain LPX(O) compound(s) and compositions were formulated for stable aqueous delivery. Formulations of emulsified LPX(O) compounds which could withstand a freeze/thaw process and/or that were suitable for delivering to animals for pharmacokinetic studies were achieved by mixing the LPX(O) compounds(s) and compositions with a solubilizing agent.

### Preparation of Alkylglycerol, Alkyldiacylglycerol and Lysoalkylphosphatidylcholine Formulations.

1. Stock solutions of individual precursor compounds (100 mg/ml) were prepared in a chloroform:methanol (1:1) solution.
2. A stock solution of purified egg yolk L-α-lecithin (Sigma-Aldrich, Saint Louis, MO, USA) (100 mg/ml) was similarly prepared in a chloroform:methanol (1:1) solution.
3. The precursor compound mix was prepared by combining appropriate volumes of the precursor compound stock (Table 15).
4. Mix of precursor compounds and lecithin stock were then aliquoted into scintillation vials (Table 15).
5. The mixture of precursor compounds and lecithin was subsequently dried under a nitrogen gas stream at 40° C.
6. Upon complete evaporation of chloroform and methanol, the dried lipids were reconstituted in 3 ml of deionized water through vigorous vortexing, followed by sonication in an Ultrasonic Cleaner water bath (Soniclean, Adelaide, SA, Australia) for 1 hour, and additional sonication using a Misonix S-4000 Sonicator (Thermo Fisher Scientific, Melbourne, VIC, Australia) for 2×30 seconds each at an amplitude of 25.

TABLE 15
Preparation of alkylglycerol, alkyldiacylglycerol and
lysoalkylphosphatidylcholine formulations.

| | | | | Volume (μl) | Volume |
|---|---|---|---|---|---|
| | Volume (μl) of | | | of precursor | (μl) of |
| | precursor compound | | | compound | lecithin |
| | stock (100 mg/ml) | | | mix | stock |
| | used to make a precursor | | | aliquoted | (100 mg/ml) |
| Precursor | compound mix | | | to | aliquoted to |
| compound | O-16:0-d2 | O-18:0-d2 | O-18:1-d2 | each vial | each vial |
| AKG Low | 452 | 598 | 350 | 140 | 140 |
| AKG High | 1517 | 2009 | 1174 | 470 | 470 |
| AKDAG- | 1202 | 1511 | 887 | 360 | 140 |
| oleic Low | | | | | |
| AKDAG- | 4007 | 5038 | 2955 | 1200 | 470 |
| oleic High | | | | | |
| AKDAG- | 1338 | 1677 | 984 | 400 | 140 |
| DHA Low | | | | | |
| AKDAG- | 4450 | 5577 | 3272 | 1330 | 470 |
| DHA High | | | | | |
| LPC(O) | 690 | 889 | 521 | 210 | 140 |
| Low | | | | | |
| LPC(O) | 2301 | 2963 | 1735 | 700 | 470 |
| High | | | | | |

### Preparation of Lysoalkylphosphatidylethanolamine Formulations

1. Stock solutions of individual precursor compounds (10 mg/ml) were prepared in a chloroform:methanol (2:1) solution*.
2. A stock solution of purified egg yolk L-α-lecithin (Sigma-Aldrich, Saint Louis, MO, USA) (100 mg/ml) was similarly prepared in a chloroform:methanol (2:1) solution.
3. The precursor compound mix was prepared by combining appropriate volumes of the precursor compound stock (Table 16).
4. Mix of precursor compounds and lecithin stock were then aliquoted into glass tubes (Table 16).
5. The mixture of precursor compounds and lecithin was subsequently dried under a nitrogen gas stream at 40° C.
6. Upon complete evaporation of chloroform and methanol, the dried lipids were reconstituted in 3 ml of deionized water through vigorous vortexing, followed by sonication in an Ultrasonic Cleaner water bath (Soniclean, Adelaide, SA, Australia) for 1 hour, and additional sonication using a Misonix S-4000 Sonicator (Thermo Fisher Scientific, Melbourne, VIC, Australia) for 2×30 seconds each at an amplitude of 25.
   *LPE(O-16:0), LPE(O-16:0)-d5 and LPE(O-18:0) solutions were briefly heated in a water bath at approximately 40° C.

Table 16. Preparation of lysoalkylphosphatidylethanolamine formulations.

TABLE 16

| | |
|---|---|
| Volume | |
| (μl) of | Volume (μl) |

| | | | |
|---|---|---|---|
| | | precursor | of lecithin |
| | Volume (μl) of precursor | compound | stock (100 |
| | compound stock (10 mg/ml) used to | mix | mg/ml) |
| Precursor | make a precursor compound mix | aliquoted | aliquoted |

| compound | O-16:0 | O-16:0-d5 | O-18:0 | O-18:1 | to each vial | to each vial |
|---|---|---|---|---|---|---|
| LPE(O) | 3224 | 3000 | 8058 | 4717 | 1900 | 140 |
| Low | | | | | | |
| LPE(O) | 17966 | 3000 | 27144 | 15890 | 6400 | 470 |
| High | | | | | | |

Without wishing to be bound by theory, it is believed that analysis of the formulations described above showed that purified egg yolk L-α-lecithin is a suitable solubilizing agent for use in preparing stable aqueous formulations that are suitable for, for example, delivery to animals and that can withstand freezing and thawing. Without wishing to be bound by theory, it is believed that formulations comprising a solubilizing agent, such as purified egg yolk L-α-lecithin, have improved stability and/or better withstand freezing and thawing compared to analogous formulations that do not contain said solubilizing agent, such as by being less prone to degradation during freezing and thawing.

Given the improved bioavailability of LPC(O)s various combinations of LPC(O)s, LPE(O)s, and LPA(O)s are contemplated for use in pharmaceutical formulations and dietary supplements. Dosages and serving sizes may be varied as appropriate to age, height, and weight of the subject.

In an embodiment, a useful human dose is 2-3 mg/kg, amounting to 100-300 mg, LPC(O) equivalents per person per day. In a further embodiment, a useful human dose is 200 mg LPC(O) equivalents per person per day.

In another embodiment, a maintenance dose may be 0.2-2 mg/kg, 25-100 mg LPC(O) equivalents per person per day. That is, for some people, a maintenance dose may be 25 mg, 50 mg or 100 mg LPC(O) equivalents per person per day.

In another embodiment, where a large elevation of plasmalogens is desirable or a rapid elevation thereof, a dose may consist of 3-25 mg/kg, 300-2000 mg LPC(O) equivalents per person per day. That is, where a large elevation of plasmalogens is desirable or a rapid elevation thereof, a dose may consist of 400 mg, 800 mg or 1600 mg LPC(O) equivalents per person per day.

In another embodiment, a dose may consist of 0.1-4000 mg LPC(O) equivalents per person per day.

The use of the terms “a,” “an,” “the,” and similar referents in the context of describing the present disclosure (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise stated. No language in the specification should be construed as indicating any non-claimed element as essential to the practice of the disclosure.

While in the foregoing specification this disclosure has been described in relation to certain embodiments thereof, and many details have been put forth for the purpose of illustration, it will be apparent to those skilled in the art that the disclosure is susceptible to additional embodiments and that certain of the details described herein can be varied considerably without departing from the basic principles of the disclosure.

All references cited herein are incorporated by reference in their entireties. The present disclosure may be embodied in other specific forms without departing from the spirit or essential attributes thereof, and, accordingly, reference should be made to the appended claims, rather than to the foregoing specification, as indicating the scope of the disclosure.

What is claimed is:

1. A composition comprising at least one compound of Formula (I) or a pharmaceutically acceptable salt thereof

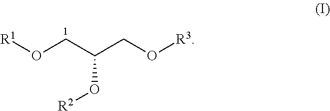

2. The composition of claim 1, wherein R³ is selected from phosphate and substituted phosphate.

3. The composition of claim 2, wherein R³ is substituted phosphate.

4. The composition of claim 3, wherein the substituted phosphate is substituted by an alkyl amine or inositol.

5. The composition of claim 4, wherein the alkyl amine is selected from

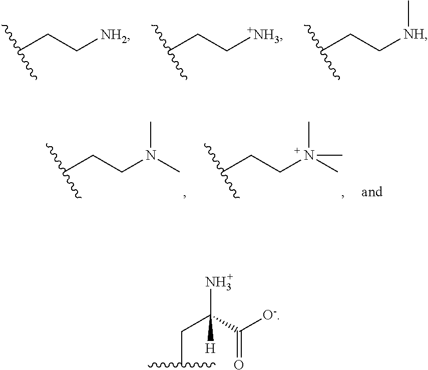

6. The composition of any one of claims 1-5, wherein R¹ and R² are each independently selected from hydrogen, an optionally substituted C₁₋₃₀ alkyl group, an optionally substituted C₂₋₃₀ alkenyl group, and an optionally substituted C₁₋₃₀ acyl group.

7. The composition of claim 6, wherein R¹ and R² are each independently selected from an optionally substituted C₁₄₋₂₄ alkyl group, an optionally substituted C₁₄₋₂₄ alkenyl group, and an optionally substituted C₁₄₋₂₄ acyl group.

8. The composition of claim 6, wherein if R² is hydrogen, R¹ is an optionally substituted C₁₋₃₀ alkyl group, an optionally substituted C₂₋₃₀ alkenyl group, or an optionally substituted C₁₋₃₀ acyl group.

9. The composition of claim 8, wherein R¹ is an optionally substituted C₁₄₋₂₄ alkyl group, an optionally substituted C₁₄₋₂₄ alkenyl group, or an optionally substituted C₁₄₋₂₄ acyl group.

10. The composition of claim 9, wherein R¹ is an optionally substituted C₁₄₋₁₈ alkyl group, an optionally substituted C₁₄₋₁₈ alkenyl group, or an optionally substituted C₁₄₋₁₈ acyl group.

11. The composition of claim 10, wherein R¹ is an unsubstituted C₁₆ alkyl group.

12. The composition of claim 10, wherein R¹ is an unsubstituted C₁₈ alkyl group.

13. The composition of claim 10, wherein R¹ is an unsubstituted C₁₈ alkenyl group.

14. The composition of claim 1, wherein the at least one compound of Formula (I) is a compound of Formula (I-A), or a pharmaceutically acceptable salt thereof

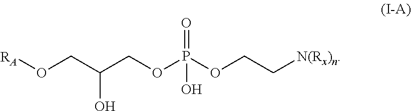

15. The composition of claim 14, wherein each Rx is independently selected from hydrogen or C₁₋₃ alkyl.

16. The composition of claim 14 or 15, wherein n is 2 or 3.

17. The composition of claim 14, wherein Rₓ is methyl, and n is 3.

18. The composition of any one of claims 14 to 17, wherein R_{A} is an optionally substituted hydrocarbon chain containing from 14 to 24 carbon atoms.

19. The composition of claim 18, wherein R_{A} is an optionally substituted hydrocarbon chain containing from 16 to 18 carbon atoms.

20. The composition of claim 19, wherein R_{A} is an optionally substituted C₁₆₋₁₈ alkyl group, an optionally substituted C₁₆₋₁₈ alkenyl group, or an optionally substituted C₁₆₋₁₈ acyl group.

21. The composition of claim 20, wherein R_{A} is an optionally substituted C₁₆₋₁₈ alkyl group.

22. The composition of claim 20, wherein R_{A} is an optionally substituted C₁₆₋₁₈ alkenyl group.

23. The composition of claim 19, wherein R_{A} is an unsubstituted C₁₆ alkyl group.

24. The composition of claim 19, wherein R_{A} is an unsubstituted C₁₈ alkyl group.

25. The composition of claim 19, wherein R_{A} is an unsubstituted C₁₈ alkenyl group.

26. The composition of any preceding claim, wherein the composition comprises a mixture of at least two compounds of Formula (I) and/or (I-A).

27. The composition of any preceding claim, wherein the composition comprises a mixture of three compounds of Formula (I) and/or (I-A).

28. The composition of claim 14, wherein the compound of Formula (I-A) is of the structure (I-A1), (I-A2), or (I-A3)

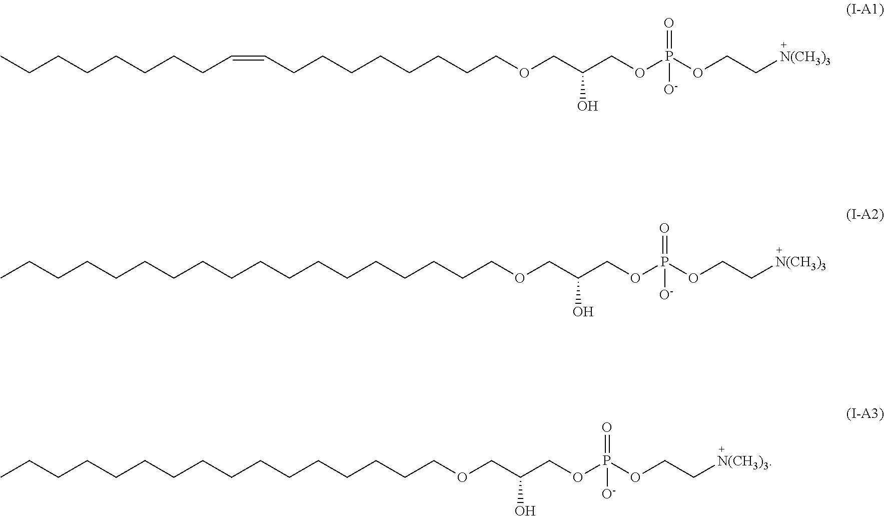

29. The composition of claim 28, wherein the composition comprises a mixture of at least two compounds selected from the group consisting of (I-A1), (I-A2), and (I-A3).

30. The composition of claim 28, wherein the composition comprises a mixture of (I-A1), (I-A2), and (I-A3).

31. The composition of claim 28, wherein the composition comprises a mixture of (I-A1), (I-A2), and (I-A3), wherein said mixture of (I-A1), (I-A2), and (I-A3) makes up at least 50% of the ether lipids in the composition on a molar percentage basis.

32. The composition of claim 28, wherein the mixture has a molar ratio of from 1.2:1 to 2.5:1 of (I-A2) to (I-A1).

33. The composition of claim 32, wherein the mixture has a molar ratio of from 1.5:1 to 2.1:1 of (I-A2) to (I-A1).

34. The composition of claim 33, wherein the mixture has a molar ratio of 1.7:1 of (I-A2) to (I-A1).

35. The composition of claim 28, wherein the mixture has a molar ratio of from 0.9:1 to 1.7:1 of (I-A2) to (I-A3).

36. The composition of claim 35, wherein the mixture has a molar ratio of from 1:1 to 1.5:1 of (I-A2) to (I-A3).

37. The composition of claim 36, wherein the mixture has a molar ratio of 1.22:1 of (I-A2) to (I-A3).

38. The composition of claim 28, wherein the mixture has a molar ratio of from 0.5:1 to 1:1 of (I-A1) to (I-A3).

39. The composition of claim 38, wherein the mixture has a molar ratio of from 0.6:1 to 0.9:1 of (I-A1) to (I-A3).

40. The composition of claim 39, wherein the mixture has a molar ratio of from 0.72:1 of (I-A1) to (I-A3).

41. The composition of claim 30, wherein the mixture has a molar percentage of (I-A1) of from 18.6% to 27.9% of (I-A2) of from 32.6% to 45.8%, and of (I-A3) of from 26.8% to 37.4%.

42. The composition of claim 30, wherein the mixture has a molar percentage of (I-A1) of 23.3%, a molar percentage of (I-A2) of 39.2%, and a molar percentage of (I-A3) of 32.1%.

43. The composition of claim 30, wherein the mixture has a molar ratio of (I-A1):(I-A2):(I-A3) of 1:1.7:1.4.

44. The composition of claim 14, wherein the compound of Formula (I-A) is of the structure (I-A4), (I-A5), or (I-A6)

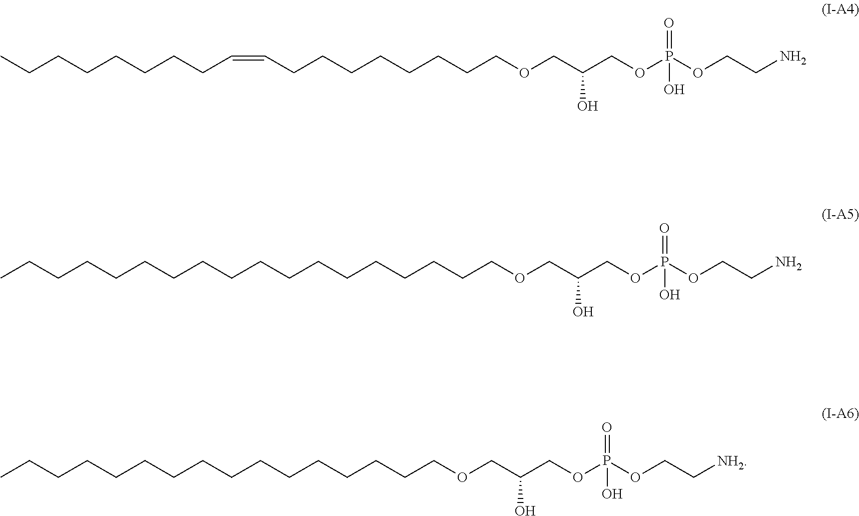 [SMILES: CCCCCCCC/C=C\CCCCCCCCOC[C@@H](O)COP(=O)(O)OCCN ; CCCCCCCCCCCCCCCCCCOC[C@@H](O)COP(=O)(O)OCCN ; CCCCCCCCCCCCCCCCOC[C@@H](O)COP(=O)(O)OCCN]

45. The composition of claim 44, wherein the composition comprises a mixture of at least two compounds selected from the group consisting of (I-A4), (I-A5), and (I-A6).

46. The composition of claim 44, wherein the composition comprises a mixture of (I-A4), (I-A5), and (I-A6).

47. The composition of claim 44, wherein the composition comprises a mixture of (I-A4), (I-A5), and (I-A6), wherein said mixture of (I-A4), (I-A5), and (I-A6) makes up at least 50% of the ether lipids in the composition on a molar percentage basis.

48. The composition of claim 44, wherein the mixture has a molar ratio of from 1.2:1 to 2.5:1 of (I-A5) to (I-A4).

49. The composition of claim 48, wherein the mixture has a molar ratio of from 1.5:1 to 2.1:1 of (I-A5) to (I-A4).

50. The composition of claim 49, wherein the mixture has a molar ratio of 1.7:1 of (I-A5) to (I-A4).

51. The composition of claim 44, wherein the mixture has a molar ratio of from 0.9:1 to 1.7:1 of (I-A5) to (I-A6).

52. The composition of claim 51, wherein the mixture has a molar ratio of from 1:1 to 1.5:1 of (I-A5) to (I-A6).

53. The composition of claim 52, wherein the mixture has a molar ratio of 1.29:1 of (I-A5) to (I-A6).

54. The composition of claim 44, wherein the mixture has a molar ratio of from 0.5:1 to 1:1 of (I-A4) to (I-A6).

55. The composition of claim 54, wherein the mixture has a molar ratio of from 0.6:1 to 0.9:1 of (I-A4) to (I-A6).

56. The composition of claim 55, wherein the mixture has a molar ratio of 0.76:1 of (I-A4) to (I-A6).

57. The composition of claim 46, wherein the mixture has a molar percentage of (I-A4) of from 18.6% to 27.9%, of (I-A5) of from 32.6% to 45.8%, and of (I-A6) of from 26.8% to 37.4%.

58. The composition of claim 57, wherein the mixture has a molar percentage of (I-A4) of 24.8%, a molar percentage of (I-A5) of 42.4%, and a molar percentage of (I-A6) of 32.8%.

59. The composition of claim 46, wherein the mixture has a molar ratio of (I-A4):(I-A5):(I-A6) of 1:1.7:1.3.

60. A formulation, such as a pharmaceutical formulation, comprising a composition as defined in any one of claims 1 to 59, and at least one excipient.

61. The formulation of claim 60, wherein the formulation is formulated for oral administration.

62. The formulation of claim 61, wherein the oral administration form is a tablet, a capsule, a solution, a mouthwash, a suspension, a powder, a gum, a confectionary, a lozenge, a sublingual delivery system, or a rapid-melt formulation.

63. The formulation of any one of claims 60 to 62, wherein the formulation is a food product.

64. The formulation of claim 63, wherein the formulation is a dietary supplement.

65. The formulation of claim 63, wherein the food product is a medical food product.

66. The formulation of claim 63, wherein the food product is infant formula.

67. The formulation according to any one of claims 60 to 66 for maintaining or modulating the levels of plasmalogens in a human subject in need thereof.

68. The formulation according to any one of claims 60 to 66 for maintaining or modulating the levels of plasmalogens in an animal subject in need thereof.

69. The formulation according to claim 68, wherein said animal is selected from domestic animals, working animals and farm animals.

70. The formulation according to any one of claims 67 to 69, wherein said maintaining or modulating requires maintaining or modulating the levels of plasmalogens at levels and/or ratios associated with a non-disease state.

71. A composition as defined in any one of claims 1 to 59, or a formulation as defined in any one of claims 60 to 70 for use in increasing levels of plasmalogen compounds in the blood or tissues of a subject.

72. The composition for use according to claim 71, wherein said subject is a human.

73. The composition for use according to claim 71, wherein said subject is an animal.

74. The composition for use according to claim 73, wherein said animal subject is selected from domestic animals, working animals and farm animals.

75. A composition as defined in any one of claims 1 to 59, or a formulation as defined in any one of claims 60 to 70 for use in therapy.

76. A composition as defined in any one of claims 1 to 59, or a formulation as defined in any one of claims 60 to 70 for use in treating a disease or disorder associated with a deficiency in plasmalogens.

77. The composition or formulation for use according to claim 76, wherein said disease or disorder is a neurological disease.

78. The composition or formulation according to claim 77, wherein said neurological disease is selected from Alzheimer's disease, Parkinson's disease, autism, amyotrophic lateral sclerosis, frontotemporal dementia, multiple sclerosis, and schizophrenia.

79. The composition or formulation for use according to claim 76, wherein said disease or disorder is a metabolic disorder.

80. The composition or formulation according to claim 79, wherein the metabolic disorder is selected from the group consisting of obesity, insulin resistance, type 2 diabetes, nonalcoholic fatty liver disease, nonalcoholic steatohepatitis, immune related disorders, cardiovascular disease, neurological diseases, cancer, myalgic encephalomyelitis/chronic fatigue syndrome, Barth syndrome, and a peroxisomal disorder.

81. The composition or formulation according to claim 80, wherein the immune related disease is selected from the group consisting of asthma, atopic dermatitis, type 1 diabetes, and infection.

82. The composition or formulation according to claim 80, wherein the cardiovascular disease is selected from the group consisting of atherosclerosis, cardiac remodeling, and hypertension.

83. The composition or formulation according to claim 80, wherein the peroxisomal disorder is a Zellweger syndrome spectrum disorder or rhizomelic chondrodysplasia punctata.

84. The composition or formulation for use according to any one of claims 71 to 83, wherein the compound of Formula (I) or (I-A) is administered in a dose of from 0.1 to 4000 mg per day.

85. The composition or formulation for use according to claim 84, wherein the compound of Formula (I) or (I-A) is administered in a dose of from 0.1 to 2000 mg per day.

86. The composition or formulation for use according to claim 85, wherein the compound of Formula (I) or (I-A) is administered in a dose of from 25 to 1600 mg per day.

87. The composition or formulation for use according to claim 86, wherein the compound of Formula (I) or (I-A) is administered in a dose of 400 mg, 800 mg, or 1600 mg per day.

88. The composition or formulation for use according to claim 86, wherein the compound of Formula (I) or (I-A) is administered in a dose of 200 mg per day.

89. The composition or formulation for use according to claim 86, wherein the compound of Formula (I) or (I-A) is administered in a dose of from 25 to 100 mg per day.

90. A method of increasing levels of plasmalogen compounds in the blood or tissues of a subject in need thereof, comprising administering to said subject an effective amount of at least one compound of Formula (I) or a pharmaceutically acceptable salt thereof and/or at least one compound of Formula (I-A) or a pharmaceutically acceptable salt thereof as defined in claims 1 and 14 respectively.

91. The method of claim 90, wherein said subject is a human.

92. The method of claim 90, wherein said subject is an animal.

93. The method of claim 92, wherein said animal subject is selected from domestic animals, working animals and farm animals.

94. The method of claim 90, wherein the bioavailability of plasmalogen compounds in the blood or tissues of said subject is improved following administration of at least one compound of Formula (I) or a pharmaceutically acceptable salt thereof and/or at least one compound of Formula (I-A) or a pharmaceutically acceptable salt thereof relative to administration of AKG (alkylglycerol).

95. A method of treating a disease or disorder associated with a deficiency in plasmalogens, comprising administering to a subject in need thereof an effective amount of at least one compound of Formula (I) or a pharmaceutically acceptable salt thereof and/or at least one compound of Formula (I-A) or a pharmaceutically acceptable salt thereof as defined in claims 1 and 14 respectively.

96. The method of claim 95, wherein said subject is a human.

97. The method of claim 95, wherein said subject is an animal.

98. The method of claim 97, wherein said animal subject is selected from domestic animals, working animals and farm animals.

99. The method of claim 95, wherein said disease or disorder is a neurological disease.

100. The method of claim 99, wherein said neurological disease is selected from the group consisting of Alzheimer's disease, Parkinson's disease, autism, amyotrophic lateral sclerosis, frontotemporal dementia, multiple sclerosis, and schizophrenia.

101. The method of claim 95, wherein said disease or disorder is a metabolic disorder.

102. The method of claim 101, wherein the metabolic disorder is selected from the group consisting of obesity, insulin resistance, type 2 diabetes, nonalcoholic fatty liver disease, nonalcoholic steatohepatitis, immune related disorders, cardiovascular disease, neurological diseases, cancer, myalgic encephalomyelitis/chronic fatigue syndrome, Barth syndrome, and a peroxisomal disorder.

103. The method of claim 102, wherein the immune related disease is selected from the group consisting of asthma, atopic dermatitis, type 1 diabetes, and infection.

104. The method of claim 102, wherein the cardiovascular disease is selected from the group consisting of atherosclerosis, cardiac remodeling, and hypertension.

105. The method of claim 102, wherein the peroxisomal disorder is a Zellweger syndrome spectrum disorder or rhizomelic chondrodysplasia punctata.

106. The method of any one of claims 90 to 105, wherein the compound of Formula (I) or (I-A) is administered in a dose of from 0.1 to 4000 mg per day.

107. The method of claim 106, wherein the compound of Formula (T) or (I-A) is administered in a dose of from 0.1 to 2000 mg per day.

108. The method of claim 107, wherein the compound of Formula (I) or (I-A) is administered in a dose of from 25 to 1600 mg per day.

109. The method of claim 108, wherein the compound of Formula (I) or (I-A) is administered in a dose of 400 mg, 800 mg, or 1600 mg per day.

110. The method of any claim 108, wherein the compound of Formula (I) or (I-A) is administered in a dose of 200 mg per day.

111. The method of claim 108, wherein the compound of Formula (I) or (I-A) is administered in a dose of from 25 to 100 mg per day.

112. The method of claim 111, wherein the compound of Formula (I) or (I-A) is administered in a dose of 25 mg, 50 mg, or 100 mg per day.

113. Use of a composition as defined in any one of claims 1 to 59, or a formulation as defined in any one of claims 60 to 70 in the manufacture of a medicament for increasing levels of plasmalogen compounds in the blood or tissues of a subject in need thereof.

114. Use of a composition as defined in any one of claims 1 to 59, or a formulation as defined in any one of claims 60 to 70 in the manufacture of a medicament for the treatment of a disease or disorder associated with a deficiency in plasmalogens.

115. Use according to claim 114, wherein said disease or disorder is a neurological disease.

116. Use according to claim 115, wherein said neurological disease is selected from the group consisting of Alzheimer's disease, Parkinson's disease, autism, amyotrophic lateral sclerosis, frontotemporal dementia, multiple sclerosis, and schizophrenia.

117. Use according to claim 116, wherein said disease or disorder is a metabolic disorder.

118. Use according to claim 117, wherein the metabolic disorder is selected from obesity, insulin resistance, type 2 diabetes, nonalcoholic fatty liver disease, nonalcoholic steatohepatitis, immune related disorders, cardiovascular disease, neurological diseases, cancer, myalgic encephalomyelitis/chronic fatigue syndrome, Barth syndrome, and a peroxisomal disorder.

119. Use according to claim 118, wherein the immune related disease is selected from the group consisting of asthma, atopic dermatitis, type 1 diabetes, and infection.

120. Use according to claim 118, wherein the cardiovascular disease is selected from the group consisting of atherosclerosis, cardiac remodeling, and hypertension.

121. Use according to claim 118, wherein the peroxisomal disorder is a Zellweger syndrome spectrum disorder or rhizomelic chondrodysplasia punctata.

122. A kit for use in the method of any one of claims 90 to 112, said kit comprising at least one compound of Formula (I) or Formula (I-A) as defined in any one of claims 1 and 14 respectively or a pharmaceutically acceptable salt thereof.

123. The composition of claim 28, wherein the at least one compound is selected from a compound of Formula (I-A1), (I-A2) or (I-A3).

124. The composition of claim 29, wherein the mixture of at least two compounds is a 50:50 mixture.

125. The composition of claim 30, wherein the mixture of (I-A1), (I-A2) and (I-A3) is selected from:

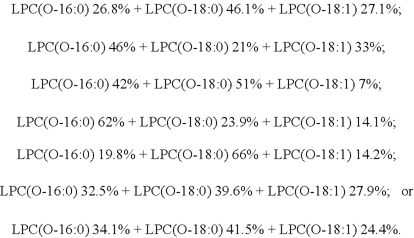

126. The composition of claim 44, wherein the at least one compound is selected from a compound of Formula (I-A4), (I-A5) or (I-A6).

127. The composition of claim 45, wherein the mixture of the at least two compounds is a 50:50 mixture.

128. The composition of claim 46, wherein the mixture of (I-A4), (I-A5) and (I-A6) is selected from:

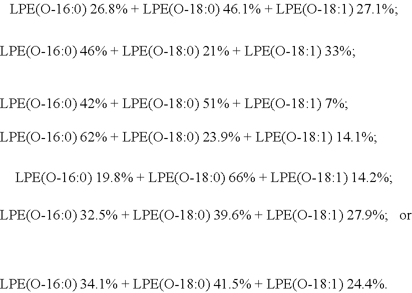

129. The composition of any one of claims 1-59 or 123-128, wherein the composition maintains or modulates the plasmalogen compound ratios observed in healthy human subjects.

130. The composition of any one of claims 1-59 or 123-128, wherein the composition results in a reduction in inflammation or the amelioration or reduction of symptoms associated with an inflammatory disease.

131. The composition of claim 130, wherein the reduction in inflammation is associated with a reduction in the levels of inflammatory cytokines.

132. The composition of any one of claims 1-59 or 123-128, wherein the composition maintains or modulates the levels of inflammatory cytokines at levels observed in healthy human subjects.

133. The composition of claim 132, wherein the inflammatory cytokine which is maintained or modulated is selected from IL-6, NFE2L2, TLR4.

134. A formulation comprising a composition as defined in any one of claims 1 to 59 or 123-128 and at least one excipient and/or solubilizing agent.

135. The formulation of claim 134, wherein the formulation is formulated for oral administration.

136. The formulation of claim 135, wherein the oral administration form is a beverage or a food product.

137. The formulation of claim 135, wherein the oral administration form is a food product.

138. The formulation of claim 135, wherein the oral administration form is infant formula.

139. The formulation of claim 134, wherein the formulation comprises a solubilizing agent.

140. The formulation of claim 139, wherein the solubilizing agent is selected from the group consisting of carboxymethylcellulose sodium, hypromellose, proline, xanthan gum, maltodextrin, alginates, waxes, lipids, oils, alcohols, sugars, microcrystalline cellulose, starches, calcium phosphates, mannitol, sorbitol, erythritol, food grade solvents, phospholipids, DMSO, ethanol, ethyl acetate, and isopropanol.

141. The formulation of claim 139, wherein the solubilizing agent is lecithin.

142. The formulation of claim 141, wherein the lecithin is purified egg yolk L-α-lecithin.

143. The formulation of claim 139, wherein the formulation has improved stability compared to a formulation comprising a composition as defined in any one of claims 1 to 59 or 123-128 that does not comprise a solubilizing agent.

144. The formulation of claim 139, wherein the formulation is less prone to degradation during freezing and thawing compared to a formulation comprising a composition as defined in any one of claims 1 to 59 or 123-128 that does not comprise a solubilizing agent.

145. The composition as defined in either one of claim 28 or claim 44, wherein the composition comprises one compound with a purity of at least 99.9%.

146. A formulation comprising a composition as defined in claim 145, and at least one excipient.

147. The formulation as defined any one of claims 134 to 138 or claim 146, wherein the formulation comprises at least one of a solubilizing agent, an emulsifier, a stabilizer, a dispersal agent, an antifoaming agent, or a diluent.

148. The formulation as defined any one of claims 134 to 138 or claims 146 to 147, wherein the formulation additionally comprises at least one solubilizing agent.

149. The formulation of claim 148, wherein the solubilizing agent is selected from the group consisting of carboxymethylcellulose sodium, hypromellose, proline, xanthan gum, maltodextrin, alginates, waxes, lipids, oils, alcohols, sugars, microcrystalline cellulose, starches, calcium phosphates, mannitol, sorbitol, erythritol, food grade solvents, phospholipids, DMSO, ethanol, ethyl acetate, and isopropanol.

150. The formulation of claim 149, wherein the solubilizing agent is selected from the group consisting of DMSO, ethanol, ethyl acetate, and isopropanol.

151. The formulation as defined any one of claims 134 to 144 or claims 146 to 150, wherein the formulation additionally comprises at least one antioxidant compound.

152. The formulation as defined any one of claims 134 to 144 or claims 146 to 151, wherein the formulation additionally comprises at least one anti-foaming agent.

153. The formulation as defined any one of claims 134 to 144 or claims 146 to 152, wherein the formulation is formulated for oral administration.

154. The formulation of claim 153, wherein the oral administration form is a tablet, a capsule, a solution, a mouthwash, a suspension, a powder, a gum, a confectionary, a lozenge, a sublingual delivery system, or a rapid-melt formulation.

155. The formulation of claim 154, wherein the oral administration form is a tablet.

156. The composition of claim 30, wherein the mixture of (I-A1), (I-A2) and (I-A3) comprises 90% w/v (I-A1).

157. The composition of claim 30, wherein the mixture of (I-A1), (I-A2) and (I-A3) comprises 90% w/v (I-A2).

158. The composition of claim 30, wherein the mixture of (I-A1), (I-A2) and (I-A3) comprises 90% w/v (I-A3).

159. The composition of claim 46, wherein the mixture of (I-A4), (I-A5) and (I-A6) comprises 90% w/v (I-A4).

160. The composition of claim 46, wherein the mixture of (I-A4), (I-A5) and (I-A6) comprises 90% w/v (I-A5).

161. The composition of claim 46, wherein the mixture of (I-A4), (I-A5) and (I-A6) comprises 90% w/v (I-A6).
